(19) United States
(12) Patent Application Publication
(10) Pub. No.: US 2021/0212998 A1
(43) Pub. Date: Jul. 15, 2021

(54) Combinations of Tie-2 Activators and Prostaglandins and Uses Thereof

(71) Applicant: Aerpio Pharmaceuticals, Inc., Cincinnati, OH (US)

(72) Inventor: Kevin Peters, Cincinnati, OH (US)

(21) Appl. No.: 17/143,394

(22) Filed: Jan. 7, 2021

Related U.S. Application Data
(60) Provisional application No. 62/958,348, filed on Jan. 8, 2020.
(60) Provisional application No. 63/089,144, filed on Oct. 8, 2020.

(51) Int. Cl. A61K 31/427; A61K 9/00; A61K 31/216; A61K 31/426; A61K 31/428; A61K 31/506; A61K 31/496; A61K 31/433; A61P 27/06

(52) U.S. Cl. CPC A61K 31/427; A61K 9/0048; A61K 31/216; A61K 31/426; A61P 27/06; A61K 31/506; A61K 31/496; A61K 31/433; A61K 31/428

(57) ABSTRACT

Described herein are combinations of compounds effective for activation of Tie-2 and inhibition of HPTPβ with a compound that causes agonism of a prostaglandin receptor. The methods and compositions of the invention can be used for the treatment of glaucoma, elevated intraocular pressure, ocular hypertension, and associated conditions by, for example, reduction of intraocular pressure in the eye.

### CROSS-REFERENCE

This application claims the benefit of U.S. Provisional Application No. 62/958,348, filed Jan. 8, 2020, and U.S. Provisional Application No. 63/089,144, filed Oct. 8, 2020, each of which is incorporated herein by reference in its entirety.

### BACKGROUND

Glaucoma is a condition characterized by damage to the optic nerve that is typically caused by elevated intraocular pressure (IOP). Intraocular pressure results from inadequate outflow of fluid in the trabecular meshwork of the eye. Ocular hypertension (OHT) occurs when the pressure in the eye surpasses the normal range with no detectable changes in vision or damage to the structure of your eyes. People with ocular hypertension have an increased risk of glaucoma. Glaucoma can lead to progressive and irreversible vision loss.

### INCORPORATION BY REFERENCE

Each patent, publication, and non-patent literature cited in the application is hereby incorporated by reference in its entirety as if each was incorporated by reference individually.

### SUMMARY

In some embodiments, the invention provides a method of reducing intraocular pressure in a subject in need thereof, the method comprising: administering to the subject a therapeutically-effective amount of a Tie-2 activator; and administering to the subject a therapeutically-effective amount of a compound that causes agonism of a prostaglandin receptor, wherein the Tie-2 activator is not an angiopoietin.

In some embodiments, the invention provides a method of reducing intraocular pressure in a subject in need thereof, the method comprising: administering to an eye of the subject a therapeutically-effective amount of a Tie-2 activator of the formula:

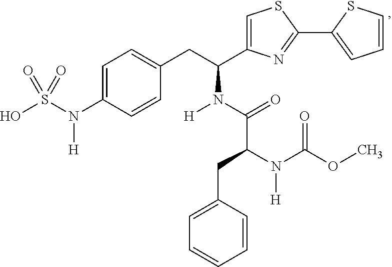 [SMILES: COC(=O)N[C@@H](Cc1ccccc1)C(=O)N[C@@H](Cc1ccc(NS(=O)(=O)O)cc1)c1csc(-c2cccs2)n1]

or a pharmaceutically-acceptable salt thereof; and administering to the eye of the subject a therapeutically-effective amount of latanoprost, wherein the Tie-2 activator is administered as part of a composition at a concentration of about 40 mg/mL, wherein the Tie-2 activator is topically administered to the eye of the subject, wherein the Tie-2 activator is administered once daily, wherein the latanoprost is administered as part of a formulation having a concentration of about 50 μg/mL, wherein the latanoprost is topically administered to the eye of the subject, wherein the latanoprost is administered once daily, and wherein the administering of the Tie-2 activator and the administering of the latanoprost reduces intraocular pressure in the subject by about 1 mmHg to about 5 mmHg.

In some embodiments, the invention provides a kit comprising: a) a Tie-2 activator; b) a compound that causes agonism of a prostaglandin receptor; and c) written instructions on use of the kit for treatment of a condition, wherein the Tie-2 activator is not an angiopoietin.

In some embodiments, the invention provides a kit comprising: a) a container, wherein the container is a dropper bottle; and b) a dosage form contained in the dropper bottle, wherein the unit dosage form comprises a Tie-2 activator and a compound that causes agonism of a prostaglandin receptor, wherein the Tie-2 activator is not an angiopoietin.

### BRIEF DESCRIPTION OF THE DRAWINGS

FIG. 1 illustrates changes in mean IOP on Day −1, Day 1, and Day 7 for subjects treated with Compound 1 in combination with once-daily prostaglandin therapy.

FIG. 2 illustrates changes in mean IOP from baseline on Day 1 and Day 7.

FIG. 3 illustrates changes in mean IOP on Day −1, Day 1, and Day 7 for subjects treated with prostaglandin and placebo.

FIG. 4 illustrates changes in conjunctival hyperemia from pre-dose baseline on Day 1 and Day 7 for subjects treated with Compound 1 in combination with prostaglandin or prostaglandin with placebo.

FIG. 5 illustrates changes in mean corrected IOP (%) from baseline on Day 1 and Day 5.

FIG. 6 illustrates changes in mean corrected IOP (mmHg) from baseline on Day 1 and Day 5.

### DETAILED DESCRIPTION

Described herein are combination therapies using a Tie-2 activator and a prostaglandin for treatment of glaucoma, elevated intraocular pressure, and ocular hypertension. A Tie-2 activator of the disclosure can activate Tie-2 signaling by promoting protein phosphorylation, such as phosphorylation of the Tie-2 protein.

Tie-2 (tyrosine kinase with immunoglobulin and epidermal growth factor homology domains 2) is a membrane receptor tyrosine kinase expressed primarily in vascular endothelial cells and a subset of hematopoietic stem cells (HSCs) and macrophages. The principle regulators of Tie-2 phosphorylation are angiopoietin 1 (Ang-1) and angiopoietin 2 (Ang-2). Ang-1 is an agonist of Tie-2, and binding of Ang-1 to Tie-2 promotes receptor phosphorylation. Ang-2 is a Tie-2 ligand that acts in a context-dependent antagonistic or agonistic manner. Binding of Ang-1 to Tie-2 increases the level of endogenous Tie-2 receptor phosphorylation and initiates downstream AKT signaling. This binding initiates a signaling cascade that can induce distinctive vascular remodeling through highly organized angiogenesis and tightening of the endothelial cell junctions (endothelium cell proximity). Within the vascular endothelium, Ang-1/Tie-2 signaling promotes endothelial cell proximity. In the HSC microenvironment, Ang-1/Tie-2 signaling contributes in a paracrine manner to the long-term repopulation of HSCs.

Under physiological conditions, the duration of Tie-2 phosphorylation is regulated by the human protein tyrosine phosphatase beta (often abbreviated as HPTPβ or HPTP beta), which removes the phosphate from the Tie-2 receptor. By inhibiting HPTPβ, the level of Tie-2 phosphorylation substantially increases, thereby restoring proper endothelial cell proximity and functions. HPTPβ plays a functional role in endothelial cell viability, differentiation, permeability, and interactions with inflammatory and endothelial support cells, such as pericytes, podocytes, and smooth muscle cells. HPTPβ and vascular endothelial protein tyrosine phosphatase (VE-PTP; the mouse orthologue of HPTPβ) are expressed in vascular endothelial cells throughout development and in the adult vasculature. A small molecule of the disclosure can activate Tie-2 downstream signaling by inhibiting HPTPβ/VE-PTP. Activation of the Angiopoietin (Angpt)/Tie-2 pathway can reduce intraocular pressure by targeting the conventional outflow pathway.

A combination therapy of the disclosure can be used to treat elevated intraocular pressure. Intraocular pressure arises from increased fluid pressure inside the eye. Pressure within the eye is maintained by the balance between the fluid entering the eye through the ciliary body and the fluid exiting the eye through the trabecular meshwork. The normal range of intraocular pressure is between about 10 mmHg to about 21 mmHg. Elevated intraocular pressure in the absence of glaucoma is referred to as ocular hypertension, which can damage the trabecular meshwork. Elevated pressure in the eye can cause damage to the optic nerve and impair central and peripheral vision.

Failure to diagnose or treat symptoms of IOP, OHT, or glaucoma can lead to permanent vision loss. The glaucoma can be, for example, primary glaucoma, pseudoexfoliative glaucoma, pigmentary glaucoma, primary juvenile glaucoma, open angle glaucoma (OAG), wide-angle glaucoma, close-angle glaucoma, acute angle-closure glaucoma, normotensive glaucoma, congenital glaucoma, acquired glaucoma, secondary glaucoma, inflammatory glaucoma, phacomorphic glaucoma, secondary angle-closure glaucoma, or neovascular glaucoma. In some cases, a Tie-2 activator of the disclosure can stabilize vasculature associated with the trabecular meshwork, thereby reducing intraocular pressure and treating ocular hypertension.

OAG is a leading cause of blindness. OAG is characterized by optic nerve and neuroretina anomalies and progressive visual field defects. Elevated IOP or OHT is the primary modifiable risk factor of OAG and reducing IOP can be effective for slowing or reducing the likelihood of vision loss due to OAG.

The conventional outflow (CO) pathway, consisting of the trabecular meshwork and Schlemm's canal, controls IOP in the eye. The CO pathway is the site of increased resistance to aqueous humor outflow in OAG. The failure to modify CO can be a contributor to continued deterioration of the CO pathway and progressive increase in IOP over time in OAG. Thus, compounds that directly target CO pathology can have improved efficacy, as monotherapies or in combination with other anti-glaucoma agents, for inhibiting progression of OAG.

Schlemm's canal develops postnatally from the intrascleral venous plexus. Schlemm's canal is considered a hybrid vessel that share functional characteristics with both lymphatic vasculature and blood endothelial vasculature. Tie-2 is expressed on both blood and lymph endothelial cells and is required for development of both vascular systems. Tie-2 is constitutively expressed and activated in the normal adult blood vasculature. The Tie-2 pathway plays a critical role in maintenance of endothelial function and vascular stability. Tie-2 is expressed and activated in Schlemm's canal endothelial cells during development and in the mature vessel. Tie-2 is most highly expressed in mature inner wall endothelium of Schlemm's canal. Disruption of the Tie-2 pathway in mice can result in failure of the formation of Schlemm's canal, increased apoptosis, and reduced formation of giant vacuoles that is consistent with an impaired CO pathway. Thus, activation of the Tie-2 pathway in Schlemm's canal can be an effective treatment for OAG by restoration of aqueous outflow via the CO pathway.

IOP is also modulated by unconventional outflow pathways which bypass the trabecular meshwork. Aqueous humor can drain through two unconventional pathways: a uveoscleral pathway where aqueous drains across the sclera to be resorbed by orbital vessels, and a uveovortex pathway where aqueous humor enters the choroid to drain through the vortex veins. In some embodiments, a compound disclosed herein can modulate IOP via the CO pathway as well as unconventional outflow pathways.

### Tie-2 Activators

Compounds disclosed herein can be effective as Tie-2 activators. The compounds can promote that activity, for example, by binding to or inhibiting HPTPβ. Thus, compounds disclosed herein can be effective as HPTPβ inhibitors. Such compounds can bind to HPTPβ, for example, by mimicking the binding mechanism of a native substrate, such as a phosphorylated compound. A compound can be a phosphate mimetic or bioisostere, for example, a sulfamic acid. The compound could also be derived from an amino acid building block or comprise an amino acid backbone for efficiency and economy of synthesis.

Non-limiting examples of Tie-2 activators include Ang-1 agonists, Ang-1 antibodies, Ang-1 recombinant proteins, Ang-1 chimeric fusion proteins (e.g., Hepta-ANG1 or C4BP-ANG1), Ang-1 mimetics, Ang-1 peptides, Ang-2 antagonists, Ang-2 antibodies, HPTPβ inhibitors, VE-PTP inhibitors, PTPRB inhibitors, TEK activators, Tie-2 peptidomimetics, MAN-01, and MAN-11.

In some embodiments, a compound disclosed herein is a compound of the formula:

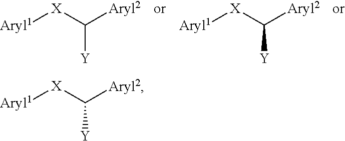

wherein:
Aryl¹ is an aryl group which is substituted or unsubstituted; Aryl² is an aryl group which is substituted or unsubstituted; X is alkylene, alkenylene, alkynylene, an ether linkage, an amine linkage, an amide linkage, an ester linkage, a thioether linkage, a carbamate linkage, a carbonate linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or

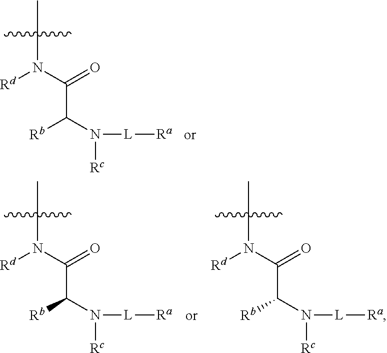

wherein:
L is alkylene, alkenylene, or alkynylene, any of which is substituted or unsubstituted, or together with the nitrogen atom to which L is bound forms an amide linkage, a carbamate linkage, or a sulfonamide linkage, or a chemical bond, or together with any of R^{a}, R^{b}, R^{c}, and R^{d} forms a ring that is substituted or unsubstituted; R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L, R^{b}, R^{c}, and R^{d} forms a ring that is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L, R^{a}, R^{c}, and R^{d} forms a ring that is substituted or unsubstituted; R^{C} is H or alkyl which is substituted or unsubstituted, or together with any of L, R^{a}, R^{b}, and R^{d} forms a ring that is substituted or unsubstituted; R^{d} is H or alkyl which is substituted or unsubstituted, or together with any of L, R^{a}, R^{b}, and R^{c} forms a ring that is substituted or unsubstituted; and R^{g} is H, alkyl, alkenyl, alkynyl, aryl, arylalkyl, heterocyclyl, heterocyclylalkyl, heteroaryl, or heteroarylalkyl, any of which is substituted or unsubstituted, or a pharmaceutically-acceptable salt, tautomer, or zwitterion thereof.

In some embodiments, Aryl¹ is substituted or unsubstituted phenyl, Aryl² is substituted or unsubstituted heteroaryl, and X is alkylene. In some embodiments, Aryl¹ is substituted phenyl, Aryl² is substituted heteroaryl, and X is methylene.

In some embodiments, a compound is of the formula:

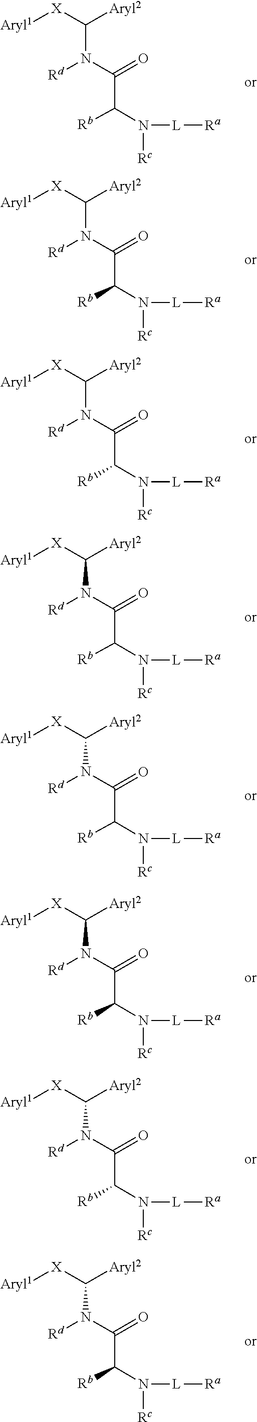

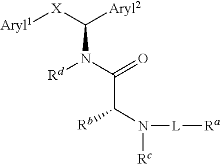

wherein Aryl¹ is para-substituted phenyl, Aryl² is substituted heteroaryl; X is methylene; L is alkylene, alkenylene, or alkynylene, any of which is substituted or unsubstituted, or together with the nitrogen atom to which L is bound forms an amide linkage, a carbamate linkage, or a sulfonamide linkage, or a chemical bond; R^{a} is H, alkyl, alkenyl, alkynyl, aryl, arylalkyl, heterocyclyl, heterocyclylalkyl, heteroaryl, or heteroarylalkyl, any of which is substituted or unsubstituted; R^{b} is H, alkyl, alkenyl, alkynyl, aryl, arylalkyl, heterocyclyl, heterocyclylalkyl, heteroaryl, or heteroarylalkyl, any of which is substituted or unsubstituted; R^{c} is H or alkyl which is substituted or unsubstituted; and R^{d} is H or alkyl which is substituted or unsubstituted.

In some embodiments, Aryl¹ is para-substituted phenyl; Aryl² is a substituted thiazole moiety; X is methylene; L together with the nitrogen atom to which L is bound forms a carbamate linkage; R^{a} is alkyl, which is substituted or unsubstituted; R^{b} is arylalkyl, which is substituted or unsubstituted; R^{C} is H; and R^{d} is H.

In some embodiments, Aryl² is:

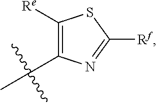

wherein R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted. In some embodiments, R^{e} is H, OH, F, Cl, Br, I, alkyl, or an alkoxy group, any of which is substituted or unsubstituted and R^{f} is alkyl, aryl, heterocyclyl, or heteroaryl, any of which is substituted or unsubstituted. In some embodiments, Aryl¹ is 4-phenylsulfamic acid; R^{a} is alkyl, which is substituted or unsubstituted; R^{b} is arylalkyl, which is substituted or unsubstituted; R^{e} is H; and R^{f} is heteroaryl. In some embodiments, Aryl¹ is 4-phenylsulfamic acid; R^{a} is alkyl; which is substituted or unsubstituted; R^{b} is arylalkyl, which is substituted or unsubstituted; R^{e} is H; and R^{f} is alkyl.

In some embodiments, Aryl² is:

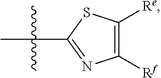

wherein R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted, R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted. In some embodiments, R^{e} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted; and R^{f} is H, OH, F, Cl, Br, I, alkyl, an alkoxy group, aryl, arylalkyl, heterocyclyl, heterocyclylalkyl, heteroaryl, or heteroarylalkyl, any of which is substituted or unsubstituted. In some embodiments, R^{e} is H, OH, F, Cl, Br, I, alkyl, or an alkoxy group, any of which is substituted or unsubstituted; and R^{f} is alkyl, aryl, heterocyclyl, or heteroaryl, any of which is substituted or unsubstituted. In some embodiments, Aryl¹ is 4-phenylsulfamic acid; R^{a} is alkyl, which is substituted or unsubstituted; R^{b} is arylalkyl, which is substituted or unsubstituted; R^{e} is H; and R^{f} is heteroaryl.

In some embodiments, a substituted phenyl group is:

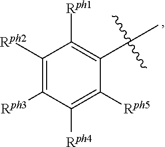

wherein:
each of R^{ph1}, R^{ph2}, R^{ph3}, R^{ph4}, and R^{ph5} is independently H, OH, F, Cl, Br, I, CN, sulfamic acid, tosylate, mesylate, triflate, besylate, alkyl, alkenyl, alkynyl, an alkoxy group, a sulfhydryl group, a nitro group, an azido group, a sulfoxide group, a sulfone group, a sulfonamide group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl.

Illustrative compounds include the following:

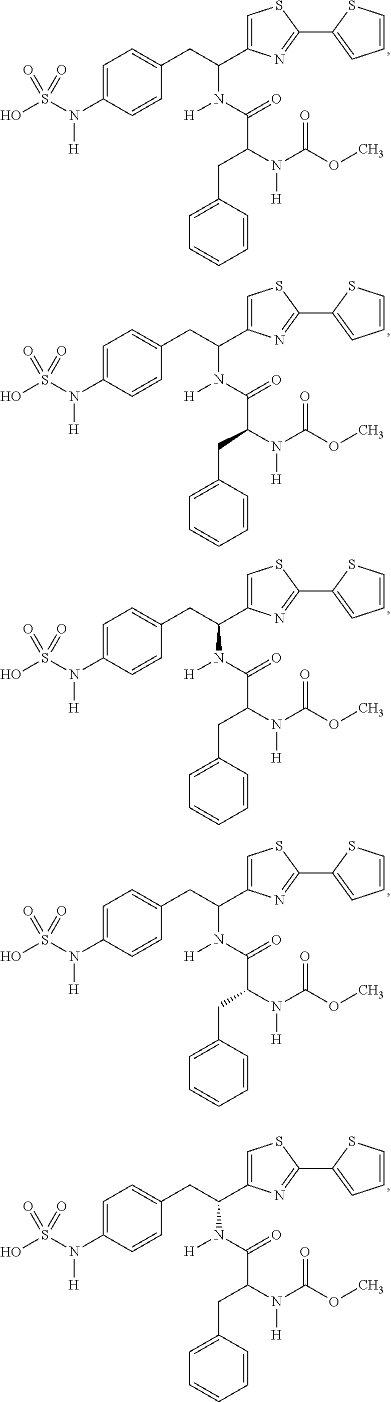

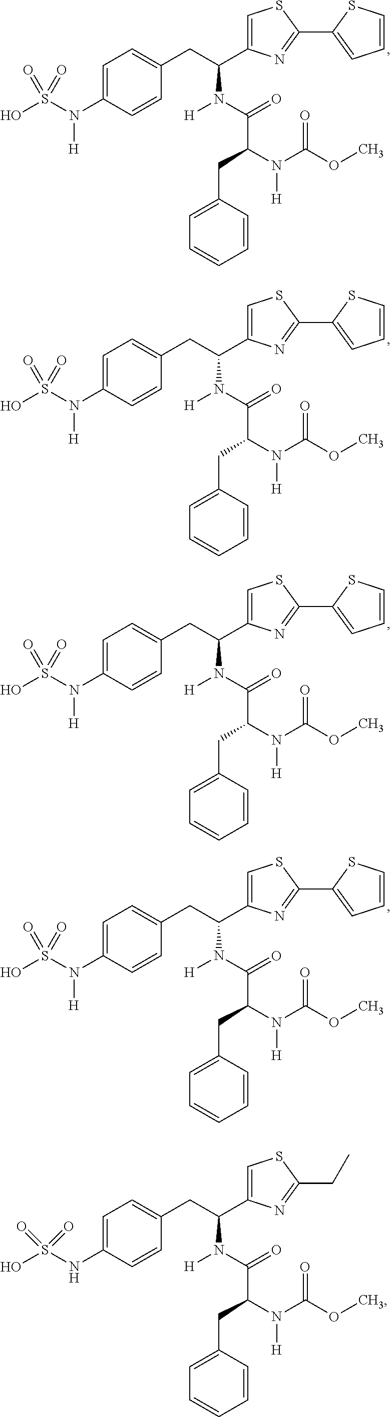

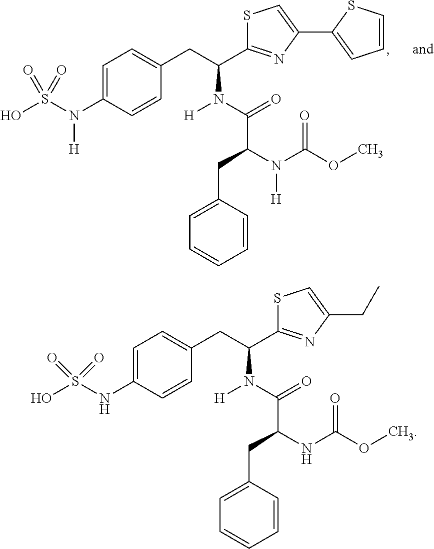 [SMILES: COC(=O)N[C@@H](Cc1ccccc1)C(=O)N[C@@H](Cc1ccc(NS(=O)(=O)O)cc1)c1nc(-c2cccs2)cs1 ; CCc1csc([C@H](Cc2ccc(NS(=O)(=O)O)cc2)NC(=O)[C@H](Cc2ccccc2)NC(=O)OC)n1]

### Optional Substituents for Chemical Groups

Non-limiting examples of optional substituents include hydroxyl groups, sulfhydryl groups, halogens, amino groups, nitro groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, and ester groups.

Non-limiting examples of alkyl and alkylene groups include straight, branched, and cyclic alkyl and alkylene groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl.

An aryloxy group can be, for example, an oxygen atom substituted with any aryl group, such as phenoxy.

An aralkyl group can be, for example, any alkyl group substituted with any aryl group, such as benzyl.

An arylalkoxy group can be, for example, an oxygen atom substituted with any aralkyl group, such as benzyloxy.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

An acyl group can be, for example, a carbonyl group substituted with hydrocarbyl, alkyl, hydrocarbyloxy, alkoxy, aryl, aryloxy, aralkyl, arylalkoxy, or a heterocycle. Non-limiting examples of acyl include acetyl, benzoyl, benzyloxycarbonyl, phenoxycarbonyl, methoxycarbonyl, and ethoxycarbonyl.

An acyloxy group can be an oxygen atom substituted with an acyl group. An ester or an ester group comprises an acyloxy group. A non-limiting example of an acyloxy group, or an ester group, is acetate.

A carbamate group can be an oxygen atom substituted with a carbamoyl group, wherein the nitrogen atom of the carbamoyl group is unsubstituted, monosubstituted, or disubstituted with one or more of hydrocarbyl, alkyl, aryl, heterocyclyl, or aralkyl. When the nitrogen atom is disubstituted, the two substituents together with the nitrogen atom can form a heterocycle.

### Pharmaceutically-Acceptable Salts

The present disclosure provides the use of pharmaceutically-acceptable salts of any compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the present disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, piprazole, imidazole, or pyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a piprazole salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

A compound herein can be a salt of an acidic group, for example:

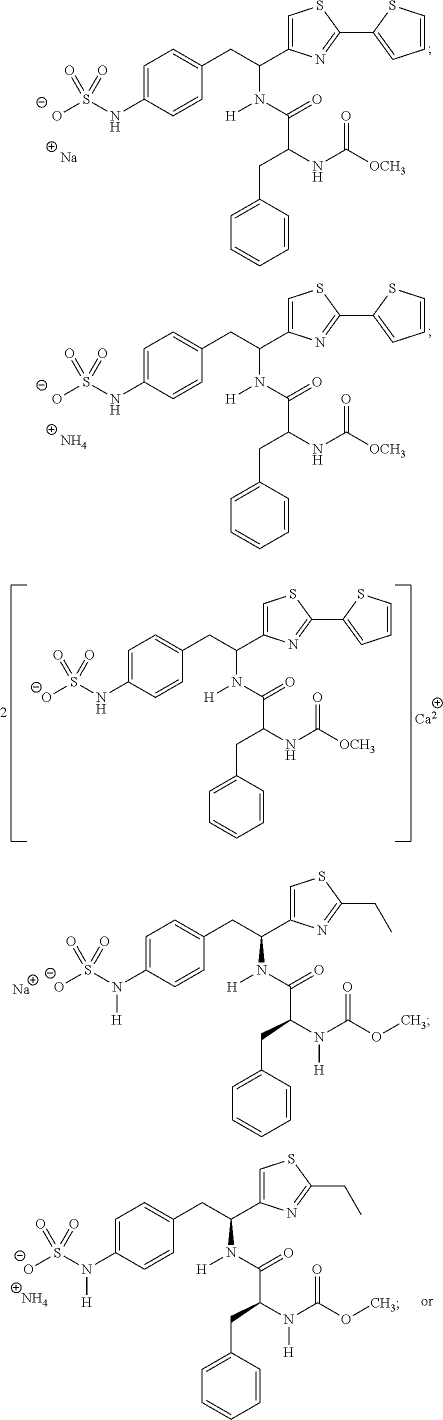

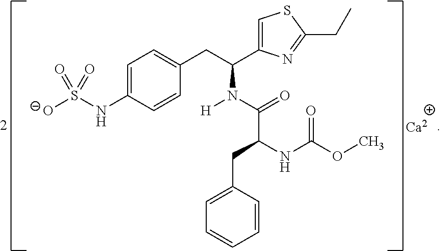

A compound herein can be a salt of a basic group formed from a strong acid, for example:

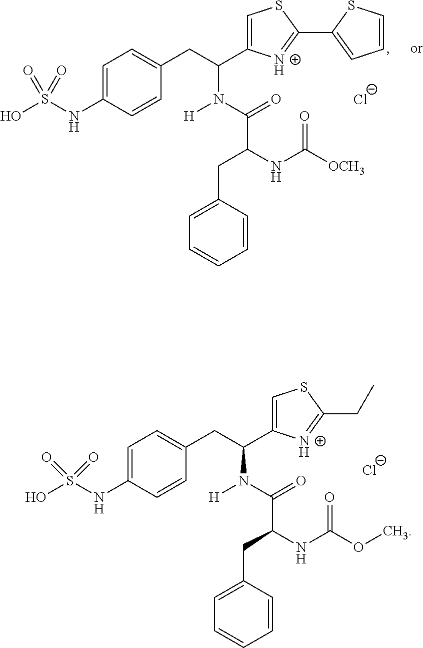 [SMILES: COC(=O)NC(Cc1ccccc1)C(=O)NC(Cc1ccc(NS(=O)(=O)O)cc1)c1csc(-c2cccs2)[nH+]1 ; [Cl-] ; CCc1[nH+]c([C@H](Cc2ccc(NS(=O)(=O)O)cc2)NC(=O)[C@H](Cc2ccccc2)NC(=O)OC)cs1 ; [Cl-]]

A compound herein can also exist in a zwitterionic form, for example:

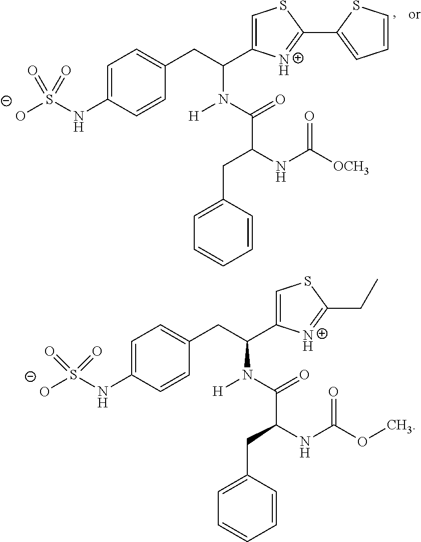 [SMILES: COC(=O)NC(Cc1ccccc1)C(=O)NC(Cc1ccc(NS(=O)(=O)[O-])cc1)c1csc(-c2cccs2)[nH+]1 ; CCc1[nH+]c([C@H](Cc2ccc(NS(=O)(=O)[O-])cc2)NC(=O)[C@H](Cc2ccccc2)NC(=O)OC)cs1]

### Formulations

A pharmaceutical composition of the disclosure can provide a therapeutically-effective amount of an activator of Tie-2 or an inhibitor of HPTPβ. A pharmaceutical composition of the disclosure can provide a therapeutically-effective amount of an activator of Tie-2.

The disclosed formulations can comprise one or more pharmaceutically-acceptable agents, which alone or in combination solubilize a compound herein or a pharmaceutically-acceptable salt thereof.

In some embodiments, a compound or pharmaceutically-acceptable salt thereof is present in a formulation in an amount of from about 0.1 μg/mL to about 100 μg/mL, from about 0.1 μg/mL to about 1 μg/mL, from about 0.1 μg/mL to about 5 μg/mL, from about 5 μg/mL to about 10 μg/mL, from about 10 μg/mL to about 15 μg/mL, from about 15 μg/mL to about 20 μg/mL, from about 20 μg/mL to about 25 μg/mL, from about 25 μg/mL to about 30 μg/mL, from about 30 μg/mL to about 35 μg/mL, from about 35 μg/mL to about 40 μg/mL, from about 40 μg/mL to about 45 μg/mL, about 45 μg/mL to about 50 μg/mL, from about 50 μg/mL to about 55 μg/mL, from about 55 μg/mL to about 60 μg/mL, from about 60 μg/mL to about 65 μg/mL, from about 65 μg/mL to about 70 μg/mL, from about 70 μg/mL to about 75 μg/mL, about 75 μg/mL to about 80 μg/mL, from about 80 μg/mL to about 85 μg/mL, from about 85 μg/mL to about 90 μg/mL, from about 90 μg/mL to about 95 μg/mL, or from about 95 μg/mL to about 100 μg/mL.

In some embodiments, a compound or pharmaceutically-acceptable salt thereof is present in a formulation in an amount of about 0.5 μg/mL, about 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, about 19 μg/mL, about 20 μg/mL, about 21 μg/mL, about 22 μg/mL, about 23 μg/mL, about 24 μg/mL, about 25 μg/mL, about 26 μg/mL, about 27 μg/mL, about 28 μg/mL, about 29 μg/mL, about 30 μg/mL, about 31 μg/mL, about 32 μg/mL, about 33 μg/mL, about 34 μg/mL, about 35 μg/mL, about 36 μg/mL, about 37 μg/mL, about 38 μg/mL, about 39 μg/mL, about 40 μg/mL, about 41 μg/mL, about 42 μg/mL, about 43 μg/mL, about 44 μg/mL, about 45 μg/mL, about 46 μg/mL, about 47 μg/mL, about 48 μg/mL, about 49 μg/mL, about 50 μg/mL, about 51 μg/mL, about 52 μg/mL, about 53 μg/mL, about 54 μg/mL, about 55 μg/mL, about 56 μg/mL, about 57 μg/mL, about 58 μg/mL, about 59 μg/mL, about 60 μg/mL, about 61 μg/mL, about 62 μg/mL, about 63 μg/mL, about 64 μg/mL, about 65 μg/mL, about 66 μg/mL, about 67 μg/mL, about 68 μg/mL, about 69 μg/mL, about 70 μg/mL, about 71 μg/mL, about 72 μg/mL, about 73 μg/mL, about 74 μg/mL, about 75 μg/mL, about 76 μg/mL, about 77 μg/mL, about 78 μg/mL, about 79 μg/mL, about 80 μg/mL, about 81 μg/mL, about 82 μg/mL, about 83 μg/mL, about 84 μg/mL, about 85 μg/mL, about 86 μg/mL, about 87 μg/mL, about 88 μg/mL, about 89 μg/mL, about 90 μg/mL, about 91 μg/mL, about 92 μg/mL, about 93 μg/mL, about 94 μg/mL, about 95 μg/mL, about 96 μg/mL, about 97 μg/mL, about 98 μg/mL, about 99 μg/mL, about 100 μg/mL, about 150 μg/mL, about 200 μg/mL, about 250 μg/mL, about 300 μg/mL, about 350 μg/mL, or about 400 μg/mL.

In some embodiments, a compound or pharmaceutically-acceptable salt thereof disclosed herein is present in a formulation in an amount of from about 0.1 mg/mL to about 100 mg/mL, from about 0.1 mg/mL to about 1 mg/mL, from about 0.1 mg/mL to about 5 mg/mL, from about 1 mg/mL to about 50 mg/mL, from about 5 mg/mL to about 10 mg/mL, from about 10 mg/mL to about 15 mg/mL, from about 15 mg/mL to about 20 mg/mL, from about 20 mg/mL to about 25 mg/mL, from about 25 mg/mL to about 30 mg/mL, from about 30 mg/mL to about 35 mg/mL, from about 35 mg/mL to about 40 mg/mL, from about 40 mg/mL to about 45 mg/mL, about 45 mg/mL to about 50 mg/mL, from about 50 mg/mL to about 55 mg/mL, from about 55 mg/mL to about 60 mg/mL, from about 60 mg/mL to about 65 mg/mL, from about 65 mg/mL to about 70 mg/mL, from about 70 mg/mL to about 75 mg/mL, about 75 mg/mL to about 80 mg/mL, from about 80 mg/mL to about 85 mg/mL, from about 85 mg/mL to about 90 mg/mL, from about 90 mg/mL to about 95 mg/mL, or from about 95 mg/mL to about 100 mg/mL. For example, a compound disclosed herein is present in a formulation in an amount of about 0.01 mg/mL, about 0.015 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, or about 1 mg/mL.

In some embodiments, a compound or pharmaceutically-acceptable salt thereof disclosed herein is present in a formulation in an amount of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, about 50 mg/mL, about 51 mg/mL, about 52 mg/mL, about 53 mg/mL, about 54 mg/mL, about 55 mg/mL, about 56 mg/mL, about 57 mg/mL, about 58 mg/mL, about 59 mg/mL, about 60 mg/mL, about 61 mg/mL, about 62 mg/mL, about 63 mg/mL, about 64 mg/mL, about 65 mg/mL, about 66 mg/mL, about 67 mg/mL, about 68 mg/mL, about 69 mg/mL, about 70 mg/mL, about 71 mg/mL, about 72 mg/mL, about 73 mg/mL, about 74 mg/mL, about 75 mg/mL, about 76 mg/mL, about 77 mg/mL, about 78 mg/mL, about 79 mg/mL, about 80 mg/mL, about 81 mg/mL, about 82 mg/mL, about 83 mg/mL, about 84 mg/mL, about 85 mg/mL, about 86 mg/mL, about 87 mg/mL, about 88 mg/mL, about 89 mg/mL, about 90 mg/mL, about 91 mg/mL, about 92 mg/mL, about 93 mg/mL, about 94 mg/mL, about 95 mg/mL, about 96 mg/mL, about 97 mg/mL, about 98 mg/mL, about 99 mg/mL, or about 100 mg/mL.

A formulation that is disclosed herein can be made more soluble by the addition of an additive or agent. The improvement of solubility of the formulation can increase by about 5%, about 10%, about 15%, about 20%, about 25%, about 30%, about 35%, about 40%, about 45%, about 50%, about 55%, about 60%, about 65%, about 70%, about 75% about 80%, about 85%, about 90%, about 95%, about 100%, about 110%, about 120%, about 130%, about 140%, about 150%, about 160%, about 170%, about 180%, about 190%, about 200%, about 225%, about 250%, about 275%, about 300%, about 325%, about 350%, about 375%, about 400%, about 450%, or about 500%.

A formulation disclosed herein can be stable for about 1 day, about 2 days, about 3 days, about 4 days, about 5 days, about 6 days, about 7 days, about 8 days, about 9 days, about 10 days, about 2 weeks, about 4 weeks, about 6 weeks, about 8 weeks, about 10 weeks, about 12 weeks, about 3 months, about 4 months, about 5 months, about 6 months, about 7 months, about 8 months, about 9 months, about 10 months, about 11 months, or about one year. A formulation disclosed herein can be stable, for example, at about 0° C., about 5° C., about 10° C., about 15° C., about 20° C., about 25° C., about 30° C., about 35° C., about 40° C., about 45° C., about 50° C., about 60° C., about 70° C., or about 80° C.

### Alcohols

A non-limiting example of a solubilizing agent includes an organic solvent. Non-limiting examples of organic solvents include alcohols, for example, C₁-C₄ linear alkyl, C₃-C₄ branched alkyl, ethanol, ethylene glycol, glycerin, 2-hydroxypropanol, maltitol, sorbitol, xylitol; substituted or unsubstituted aryl, and benzyl alcohol.

### Cyclodextrins

Non-limiting examples of cyclodextrins include β-cyclodextrin, methyl-β-cyclodextrin, 2-hydroxypropyl-β-cyclodextrin, sulfobutyl ether-β-cyclodextrin sodium salt, hydroxyethyl-β-cyclodextrin (HEβCD), heptakis (2,6-di-O-methyl)-β-cyclodextrin (DMβCD), 2-hydroxypropyl-β-cyclodextrin, γ-cyclodextrin, and 2-hydroxypropyl-γ-cyclodextrin. A cyclodextrin can possess a large cyclic structure with a channel passing through the center of the structure. The interior of the cyclodextrin can be hydrophobic, and interact favorably with hydrophobic molecules. The exterior of the cyclodextrin can be highly hydrophilic owing to the several hydroxyl groups exposed to bulk solvent. Capture of a hydrophobic molecule, such as a compound disclosed herein, in the channel of the cyclodextrin can result in the formation of a complex stabilized by non-covalent hydrophobic interactions. The complex can be soluble in water, and carry the captured hydrophobic molecule into the bulk solvent.

The disclosed solubilizing systems comprise 2-hydroxypropyl-β-cyclodextrin (HPβCD). 2-Hydroxypropyl-3-cyclodextrin [CAS No. 128446-35-5] is commercially available as Cavitron™. 2-Hydroxypropyl-β-cyclodextrin, also known as hydroxypropyl-β-cyclodextrin, 2-hydroxypropyl-beta-cyclodextrin, hydroxypropyl-beta-cyclodextrin, or HPBCD, can be represented by either of the following formulae:

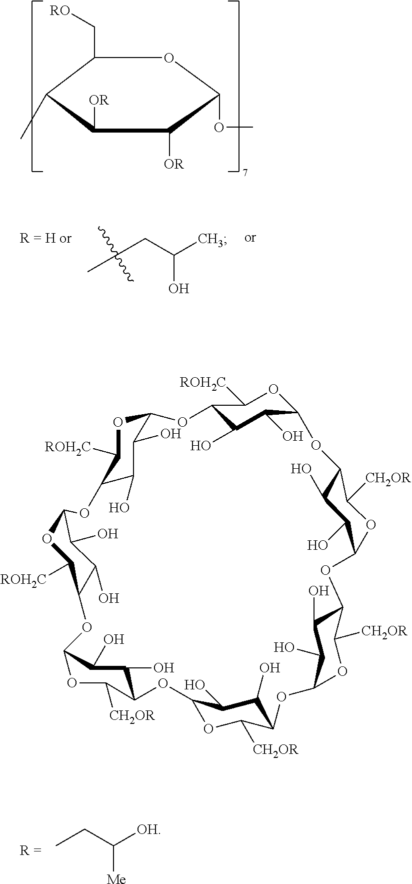

The average molecular weight of Cavitron™, is approximately 1396 Da, wherein the average degree of substitution is from about 0.5 to about 1.3 units of 2-hydroxypropyl per ring glucose unit.

The disclosed solubilizing systems comprise 2-hydroxypropyl-γ-cyclodextrin (HPγCD). 2-Hydroxypropyl-γ-cyclodextrin [CAS No. 128446-34-4], also known as hydroxypropyl-γ-cyclodextrin, 2-hydroxypropyl-gamma-cyclodextrin, hydroxypropyl-gamma-cyclodextrin, or HPGCD, can be represented by the following formula:

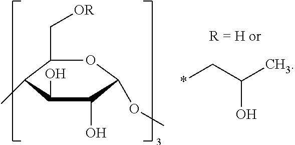

In one embodiment, a formulation disclosed herein can comprise a ratio of about 20 parts of a compound herein or a pharmaceutically-acceptable salt thereof to about 1 part solubilizing system (about 20:about 1), to about 1 part of the compound herein or a pharmaceutically-acceptable salt thereof to about 20 parts solubilizing system (about 1:about 20). For example, a formulation containing about 100 mg of a compound herein or a pharmaceutically-acceptable salt thereof can contain from about 5 mg to about 2000 mg of a solubilizing agent, such as a cyclodextrin. In another embodiment, the ratio can be based on number, or moles, or compound compared to number, or moles, of the solubilizing system.

The following are non-limiting examples of ratios of a compound herein and a solubilizing agent, such as a cyclodextrin. The following examples alternatively describe the ratio of a solubilizing agent, such as a cyclodextrin, and a compound herein. The ratio can be: about 20:about 1; about 19.9:about 1; about 19.8:about 1; about 19.7:about 1; about 19.6:about 1; about 19.5:about 1; about 19.4:about 1; about 19.3:about 1; about 19.2:about 1; about 19.1:about 1; about 19:about 1; about 18.9:about 1; about 18.8:about 1; about 18.7:about 1; about 18.6:about 1; about 18.5:about 1; about 18.4:about 1; about 18.3:about 1; about 18.2:about 1; about 18.1:about 1; about 18:about 1; about 17.9:about 1; about 17.8:about 1; about 17.7:about 1; about 17.6:about 1; about 17.5:about 1; about 17.4:about 1; about 17.3:about 1; about 17.2:about 1; about 17.1:about 1; about 17:about 1; about 16.9:about 1; about 16.8:about 1; about 16.7:about 1; about 16.6:about 1; about 16.5:about 1; about 16.4:about 1; about 16.3:about 1; about 16.2:about 1; about 16.1:about 1; about 16: about 1; about 15.9:about 1; about 15.8:about 1; about 15.7:about 1; about 15.6:about 1; about 15.5:about 1; about 15.4:about 1; about 15.3:about 1; about 15.2:about 1; about 15.1:about 1; about 15:about 1; about 14.9:about 1; about 14.8:about 1; about 14.7:about 1; about 14.6:about 1; about 14.5:about 1; about 14.4:about 1; about 14.3:about 1; about 14.2:about 1; about 14.1:about 1; about 14:about 1; about 13.9:about 1; about 13.8:about 1; about 13.7:about 1; about 13.6:about 1; about 13.5:about 1; about 13.4:about 1; about 13.3:about 1; about 13.2:about 1; about 13.1:about 1; about 13:about 1; about 12.9:about 1; about 12.8:about 1; about 12.7:about 1; about 12.6:about 1; about 12.5:about 1; about 12.4:about 1; about 12.3:about 1; about 12.2 about 1; about 12.1:about 1; about 12:about 1; about 11.9 about 1; about 11.8:about 1; about 11.7:about 1; about 11.6:about 1; about 11.5:about 1; about 11.4:about 1; about 11.3:about 1; about 11.2:about 1; about 11.1:about 1; about 11:about 1; about 10.9:about 1; about 10.8:about 1; about 10.7:about 1; about 10.6:about 1; about 10.5:about 1; about 10.4:about 1; about 10.3:about 1; about 10.2:about 1; about 10.1:about 1; about 10:about 1; about 9.9:about 1; about 9.8:about 1; about 9.7:about 1; about 9.6: about 1; about 9.5:about 1; about 9.4:about 1; about 9.3:about 1; about 9.2:about 1; about 9.1:about 1; about 9:about 1; about 8.9:about 1; about 8.8:about 1; about 8.7:about 1; about 8.6:about 1; about 8.5:about 1; about 8.4:about 1; about 8.3:about 1; about 8.2:about 1; about 8.1:about 1; about 8:about 1; about 7.9:about 1; about 7.8:about 1; about 7.7:about 1; about 7.6:about 1; about 7.5:about 1; about 7.4:about 1; about 7.3:about 1; about 7.2:about 1; about 7.1:about 1; about 7:about 1; about 6.9:about 1; about 6.8:about 1; about 6.7:about 1; about 6.6:about 1; about 6.5:about 1; about 6.4:about 1; about 6.3:about 1; about 6.2:about 1; about 6.1:about 1; about 6:about 1; about 5.9:about 1; about 5.8:about 1; about 5.7:about 1; about 5.6:about 1; about 5.5:about 1; about 5.4:about 1; about 5.3:about 1; about 5.2:about 1; about 5.1:about 1; about 5:about 1; about 4.9:about 1; about 4.8:about 1; about 4.7:about 1; about 4.6:about 1; about 4.5:about 1; about 4.4:about 1; about 4.3:about 1; about 4.2:about 1; about 4.1:about 1; about 4:about 1; about 3.9:about 1; about 3.8:about 1; about 3.7:about 1; about 3.6:about 1; about 3.5:about 1; about 3.4:about 1; about 3.3:about 1; about 3.2:about 1; about 3.1:about 1; about 3:about 1; about 2.9:about 1; about 2.8:about 1; about 2.7:about 1; about 2.6:about 1; about 2.5:about 1; about 2.4:about 1; about 2.3:about 1; about 2.2:about 1; about 2.1:about 1; about 2:about 1; about 1.9:about 1; about 1.8:about 1; about 1.7:about 1; about 1.6:about 1; about 1.5:about 1; about 1.4:about 1; about 1.3:about 1; about 1.2:about 1; about 1.1:about 1; or about 1:about 1.

### Polyvinylpyrrolidione

Another non-limiting example of a solubilizing agent is polyvinylpyrrolidone (PVP), having the formula:

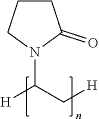

wherein the index n is from about 40 to about 200. PVPs can have an average molecular weight from about 5500 g/mol to about 28,000 g/mol. One non-limiting example is PVP-10, having an average molecular weight of approximately 10,000 g/mol.

### Polyalkyleneoxides and Ethers Thereof

Another non-limiting example of solubilizing agents includes polyalkyleneoxides, and polymers of alcohols or polyols. Polymers can be mixed, or contain a single monomeric repeat subunit. For example, polyethylene glycols (PEG) having an average molecular weight of from about 200 to about 20,000, for example, PEG 200, PEG 400, PEG 600, PEG 1000, PEG 1450, PEG 1500, PEG 4000, PEG 4600, and PEG 8000. In a same embodiment, a composition comprises one or more polyethylene glycols chosen from PEG 400, PEG 1000, PEG 1450, PEG 4600 and PEG 8000.

Other polyalkyleneoxides are polypropylene glycols having the formula:

HO[CH(CH₃)CH₂O]ₓH

wherein the index x represents the average number of propyleneoxy units in the polymer. The index x can be represented by a whole number or a fraction. For example, a polypropylene glycol having an average molecular weight of 8,000 g/mol (PEG 8000) can be represented by the formulae:

HO[CH(CH₃)CH₂O]₁₃₈H or HO[CH(CH₃)CH₂O]_{137.6}H

or the polypropylene glycol can be represented by the common, short hand notation: PEG 8000.

Another example of polypropylene glycols can have an average molecular weight from about 1,200 g/mol to about 20,000 g/mol, i.e., a polypropylene glycol having an average molecular weight of about 8,000 g/mol, for example, PEG 8000.

Another solubilizing agent is Polysorbate 80 (Tween™ 80), which is an oleate ester of sorbitol and its anhydrides copolymerized with approximately 20 moles of ethylene oxide for each mole of sorbitol and sorbitol anhydrides. Polysorbate 80 is made up of sorbitan mono-9-octadecanoate poly(oxy-1,2-ethandiyl) derivatives.

Solubilizing agents also include poloxamers having the formula:

HO(CH₂CH₂)_{y1}(CH₂CH₂CH₂O)_{y2}(CH₂CH₂O)_{y3}OH

which are nonionic block copolymers composed of a polypropyleneoxy unit flanked by two polyethyleneoxy units. The indices y¹, y², and y³ have values such that the poloxamer has an average molecular weight of from about 1000 g/mol to about 20,000 g/mol.

### Excipients

A pharmaceutical composition of the present disclosure can be a combination of any pharmaceutical compounds described herein with other chemical components, such as carriers, stabilizers, diluents, dispersing agents, suspending agents, thickening agents, or excipients. The pharmaceutical composition facilitates administration of the compound to an organism. Pharmaceutical compositions can be administered in therapeutically-effective amounts as pharmaceutical compositions by various forms and routes including, for example, intravenous, intravitreal, intranasal, intratracheal, intrapulmonary, transmucosal, subcutaneous, intramuscular, oral, rectal, aerosol, parenteral, ophthalmic, pulmonary, transdermal, vaginal, otic, nasal, and topical administration.

A pharmaceutical composition can be administered in a local or systemic manner, for example, via injection of the compound directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

A pharmaceutical composition can be administered at any time of day, for example, in the morning, in the afternoon, or in the evening. In some embodiments, pharmaceutical compositions administered at a specific time of day results in superior therapeutic effects. In some embodiments, pharmaceutical compositions described herein are administered in the evening.

For oral administration, pharmaceutical compositions can be formulated readily by combining the active compounds with pharmaceutically-acceptable carriers or excipients. Such carriers can be used to formulate tablets, powders, pills, dragees, capsules, liquids, gels, syrups, elixirs, slurries, suspensions, and the like, for oral ingestion by a subject.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used, which can contain an excipient, such as gum arabic, talc, polyvinylpyrrolidone, carbopol gel, polyethylene glycol, or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. A gelatin can be alkaline-processed. The push-fit capsules can contain the active ingredients in admixture with filler, such as lactose, binders, such as starches or lubricants, such as talc or magnesium stearate, and stabilizers. In soft capsules, the active compounds can be dissolved or suspended in suitable liquids, such as fatty oils, liquid paraffin, or liquid polyethylene glycols. Stabilizers can be added. All formulations for oral administration are provided in dosages suitable for such administration.

For buccal or sublingual administration, the compositions can be tablets, lozenges, or gels.

Parenteral injections can be formulated for bolus injection or continuous infusion. The pharmaceutical compositions can be in a form suitable for parenteral injection as a sterile suspension, solution, or emulsion in oily or aqueous vehicles, and can contain formulatory agents, such as suspending, stabilizing, or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils, such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers, and preservatives.

Formulations suitable for transdermal administration of the active compounds can employ transdermal delivery devices and transdermal delivery patches, and can be lipophilic emulsions or buffered aqueous solutions, dissolved or dispersed in a polymer or an adhesive. Such patches can be constructed for continuous, pulsatile, or on-demand delivery of pharmaceutical compounds. Transdermal delivery can be accomplished by means of iontophoretic patches. Additionally, transdermal patches can provide controlled delivery. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically-acceptable solvents to assist passage through the skin. For example, transdermal devices can be in the form of a bandage comprising a backing member, a reservoir containing compounds and carriers, a rate controlling barrier to deliver the compounds to the skin of the subject at a controlled and predetermined rate over a prolonged period of time, and adhesives to secure the device to the skin or the eye.

For administration by inhalation, the active compounds can be in a form as an aerosol, a vapor, a mist, or a powder. Inhalation can occur through by nasal delivery, oral delivery, or both. Pharmaceutical compositions are conveniently delivered in the form of an aerosol spray presentation from pressurized packs, a nebulizer, or an atomizer, with the use of a suitable propellant, for example, dichlorodifluoromethane, trichlorofluoromethane, dichlorotetrafluoroethane, difluoroethane, carbon dioxide, nitrogen, oxygen, or other suitable gas. Nebulizers are available as jet nebulizers, ultrasonic nebulizers, or vibrating mesh nebulizers. Jet nebulizers operate by compressed air. Ultrasonic nebulizers use a piezoelectric transducer to create droplets from an open liquid reservoir. Vibrating mesh nebulizers use vibrating perforated membranes (mesh) actuated by an annular piezoelectric element. The holes in the membrane have a wide cross-sectional diameter on the liquid supply side and a narrow cross-section diameter on the side from where the droplets emerge.

In the case of a pressurized aerosol, the dosage unit can be determined by providing a valve to deliver a metered amount, for example, using a metered dose inhaler (MDI). Capsules and cartridges of, for example, gelatin for use in an inhaler or insufflator can be formulated to contain a powder mix of the compounds and a suitable powder base, such as lactose or starch. Powder aerosols can be administered by dry powder inhalers (DPI). Aerosols can also be administered by a facemask interface, which can be a preferred delivery route for pediatric patients less than 5 years of age. Selection of a suitable inhalation device depends on favors, such as nature of the active compound and its formulation, the delivery site of interest, and pathophysiology of the lung.

Nasal or intranasal administration involves insufflation of compounds through the nose, which includes nasal drops and nasal sprays. This route of administration can result in local and/or systemic effects. Inhaler or insufflator devices can be used for nose-to-lung delivery of compounds described herein.

The compounds can also be formulated in rectal compositions, such as enemas, rectal gels, rectal foams, rectal aerosols, suppositories, jelly suppositories, or retention enemas containing conventional suppository bases, such as cocoa butter or other glycerides, as well as synthetic polymers, such as polyvinylpyrrolidone and PEG. In suppository forms of the compositions, a low-melting point wax, such as a mixture of fatty acid glycerides or cocoa butter can be used.

In practicing the methods of treatment or use provided herein, therapeutically-effective amounts of the compounds described herein are administered in pharmaceutical compositions to a subject having a disease or condition to be treated. In some embodiments, the subject is a mammal, such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a compound described herein can be manufactured, for example, by mixing, dissolving, granulating, dragee-making, levigating, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. The methods and pharmaceutical compositions described herein include the use of crystalline forms (also known as polymorphs), and active metabolites of these compounds having the same type of activity.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, cachets, and suppositories. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions, and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the present disclosure include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

The individual dose administered to a subject can be about 0.5 μg, about 1 μg, about 1.1 μg, about 1.2 μg, about 1.3 μg, about 1.4 μg, about 1.5 μg, about 1.6 μg, about 1.7 μg, about 1.8 μg, about 1.9 μg, about 2 μg, about 3 μg, about 4 μg, about 5 μg, about 6 μg, about 7 μg, about 8 μg, about 9 μg, about 10 μg, about 20 μg, about 30 μg, about 40 μg, about 50 μg, about 60 μg, about 70 μg, about 80 μg, about 90 μg, about 100 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, or about 5 mg of a compound of the present disclosure.

Non-limiting examples of pharmaceutically-acceptable excipients suitable for use in the present disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, antimicrobial agents, plant cellulosic material and spheronization agents, and any combination thereof.

A composition of the present disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that drug release rates and drug release profiles can be matched to physiological and chronotherapeutic requirements or, alternatively, has been formulated to effect release of a drug at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

The disclosed compositions can optionally comprise from about 0.001% to about 0.005% weight by volume pharmaceutically-acceptable preservatives. One non-limiting example of a suitable preservative is benzyl alcohol.

In some embodiments, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

A disclosed composition can be administered at any dose frequency. In some embodiments, a compound described herein is administered once daily, twice daily, three times daily, four times daily, weekly, twice weekly, once every two weeks, or monthly.

A disclosed composition can be administered at any volume. In some embodiments, a composition described herein is administered as a topical eye drop, each drop having a volume of about 20 μL, about 25 μL, about 30 μL, about 31 μL, about 32 μL, about 33 μL, about 34 μL, about 35 μL, about 40 μL, about 45 μL, about 50 μL, about 60 μL, about 70 μL, about 80 μL, about 90 μL, or about 100 μL. In some embodiments, an eye drop is administered to the conjunctival sac of an affected eye.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

The disclosed methods include administration of a Tie-2 activator, or a pharmaceutically-acceptable salt thereof, in combination with a pharmaceutically-acceptable carrier. The carrier can be selected to minimize any degradation of the active ingredient and to minimize any adverse side effects in the subject.

The disclosed methods include administration of a Tie-2 activator, or a pharmaceutically-acceptable salt thereof, in combination with a pharmaceutically-acceptable carrier. The carrier can be selected to minimize any degradation of the active ingredient and to minimize any adverse side effects in the subject.

The Tie-2 activator or a pharmaceutically-acceptable salt thereof herein can be conveniently formulated into pharmaceutical compositions composed of one or more pharmaceutically-acceptable carriers. See e.g., Remington's Pharmaceutical Sciences, latest edition, by E.W. Martin Mack Pub. Co., Easton, Pa., which discloses typical carriers and conventional methods of preparing pharmaceutical compositions that can be used in conjunction with the preparation of formulations of the compound described herein and which is incorporated by reference herein. Such pharmaceuticals can be standard carriers for administration of compositions to humans and non-humans, including solutions, such as sterile water, saline, and buffered solutions at physiological pH. Other compositions can be administered according to standard procedures. For example, pharmaceutical compositions can also include one or more additional active ingredients, such as antimicrobial agents, anti-inflammatory agents, and anesthetics.

Non-limiting examples of pharmaceutically-acceptable carriers include saline solution, Ringer's solution, and dextrose solution. The pH of the solution can be from about 5 to about 8, and can be from about 7 to about 7.5. Further carriers include sustained release preparations, such as semipermeable matrices of solid hydrophobic polymers containing the Tie-2 activator or a pharmaceutically-acceptable salt thereof, where the matrices are in the form of shaped articles, such as films, liposomes, microparticles, and microcapsules.

The disclosed methods relate to administering the Tie-2 activator or a pharmaceutically-acceptable salt thereof as part of a pharmaceutical composition. The disclosed methods relate to administering the HPTPβ inhibitor or a pharmaceutically-acceptable salt thereof as part of a pharmaceutical composition. In various embodiments, compositions of the present disclosure can comprise a liquid comprising an active agent in solution, in suspension, or both. Liquid compositions can include gels. In one embodiment, the liquid composition is aqueous. Alternatively, the composition can take form of an ointment. In another embodiment, the composition is an in situ gellable aqueous composition. In some embodiments, the composition is an in situ gellable aqueous solution.

Pharmaceutical formulations can include additional carriers, as well as thickeners, diluents, buffers, preservatives, and surface active agents in addition to the compounds disclosed herein. Pharmaceutical formulations can also include one or more additional active ingredients, such as antimicrobial agents, anti-inflammatory agents, anesthetics, and the like.

An excipient can fill a role as simple and direct as being an inert filler, or an excipient as used herein can be part of a pH stabilizing system or coating to insure delivery of the ingredients safely to the stomach.

The Tie-2 activator or HPTPβ inhibitor, or a pharmaceutically-acceptable salt thereof, can also be present in liquids, emulsions, or suspensions for delivery of active therapeutic agents in aerosol form to cavities of the body, such as the nose, throat, or bronchial passages. The ratio of Tie-2 activator or a pharmaceutically-acceptable salt thereof to the other compounding agents in these preparations can vary as the dosage form requires.

Depending on the intended mode of administration, the pharmaceutical compositions administered as part of the disclosed methods can be in the form of solid, semi-solid or liquid dosage forms, such as, for example, tablets, suppositories, pills, capsules, powders, liquids, suspensions, lotions, creams, gels, for example, in a unit dosage form suitable for single administration of a precise dosage. The compositions can contain, as noted above, an effective amount of the Tie-2 activator or a pharmaceutically-acceptable salt thereof in combination with a pharmaceutically-acceptable carrier and, in addition, can include other medicinal agents, pharmaceutical agents, carriers, adjuvants, diluents, etc.

For solid compositions, nontoxic solid carriers include, for example, pharmaceutical grades of mannitol, lactose, starch, magnesium stearate, sodium saccharin, talc, cellulose, glucose, sucrose, and magnesium carbonate. In one embodiment, a composition comprising the Tie-2 activator or a pharmaceutically-acceptable salt thereof in an amount of approximately 4 mg per 0.1 mL liquid is prepared. The liquid phase comprises sterile water and an appropriate amount of a saccharide or polysaccharide.

### Pharmaceutical Compositions

Pharmaceutical compositions containing the compounds described herein can be administered for prophylactic or therapeutic treatments. Compositions can contain any number of active agents. In therapeutic applications, the compositions can be administered to a subject already suffering from a disease or condition, in an amount sufficient to cure or at least partially arrest the symptoms of the disease or condition, or to cure, heal, improve, reduce, lessen or ameliorate the disease or condition. Compounds can also be administered to lessen or reduce a likelihood of developing, contracting, or worsening a condition. Amounts effective for this use can vary based on the severity and course of the disease or condition, previous therapy, the subject's health status, weight, response to the drugs, and the judgment of the treating physician.

Multiple therapeutic agents can be administered in any order or simultaneously. If simultaneously, the multiple therapeutic agents can be provided in a single, unified form, or in multiple forms, for example, as multiple separate pills or injections. The compounds can be packed together or separately, in a single package or in a plurality of packages. One or all of the therapeutic agents can be given in multiple doses. If not simultaneous, the timing between the multiple doses can vary.

Compounds and compositions described herein can be packaged as a kit. In some embodiments, the present disclosure provides a kit comprising a compound disclosed herein, or a pharmaceutically-acceptable salt thereof, and written instructions on use of the kit in the treatment of a condition described herein. In some embodiments, the present disclosure provides a kit comprising a compound disclosed herein, or a pharmaceutically-acceptable salt thereof, an antibody, and written instructions on use of the kit in the treatment of a condition described herein.

The compounds described herein can be administered before, during, or after the occurrence of a disease or condition, and the timing of administering the composition containing a compound can vary. For example, the compounds can be used as a prophylactic and can be administered continuously to subjects with a propensity to conditions or diseases in order to lessen or reduce a likelihood of the occurrence of the disease or condition. The compounds and compositions can be administered to a subject during or as soon as possible after the onset of the symptoms. The administration of the compounds can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for parenteral injection can be presented in unit dosage form, for example, in ampoules, or in multi-dose containers with a preservative.

A compound described herein can be administered to a subject at a dose of from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, or from about 250 mg to about 300 mg.

A compound described herein can be administered to a subject at a dose of about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 225 mg, about 250 mg, or about 300 mg.

A compound described herein can be administered to a subject at a dose of about 0.1 μg, about 0.2 μg, about 0.3 μg, about 0.4 μg, about 0.5 μg, about 0.6 μg, about 0.7 μg, about 0.8 about 0.9 μg, about 1 μg, about 1.1 μg, about 1.2 μg, about 1.3 μg, about 1.4 μg, about 1.5 about 1.6 μg, about 1.7 μg, about 1.8 μg, about 1.9 μg, about 2.0 μg, about 2.1 μg, about 2.2 about 2.3 μg, about 2.4 μg, about 2.5 μg, about 2.6 μg, about 2.7 μg, about 2.8 μg, about 2.9 about 3.0 μg, about 3.1 μg, about 3.2 μg, about 3.3 μg, about 3.4 μg, about 3.5 μg, about 3.6 about 3.7 μg, about 3.8 μg, about 3.9 μg, about 4.0 μg, about 4.1 μg, about 4.2 μg, about 4.3 about 4.4 μg, about 4.5 μg, about 4.6 μg, about 4.7 μg, about 4.8 μg, about 4.9 μg, or about 5.0

### Combination Therapy

In some embodiments, a Tie-2 activator or HPTPβ inhibitor can be administered in conjunction with one or more additional agents that modulate intraocular pressure or treat glaucoma. Additional agents include prostaglandin agonists, β-adrenoceptor antagonists (beta blockers), carbonic anhydrase inhibitors, α-adrenergic agonists, Rho kinase inhibitors, miotic agents, and cholinergic agonists. Beta blockers can decrease production of ocular fluid. Systemic side effects of beta blockers can be minimized by closing the eyes following application or using a technique called punctal occlusion that prevents the drug from entering the tear drainage duct and systemic circulation. Alpha adrenergic agonists can both decrease production of ocular fluid and increase drainage. Carbonic anhydrase inhibitors reduce intraocular pressure by decreasing the production of intraocular fluid. Rho kinase inhibitors can increase the drainage of intraocular fluid.

Non-limiting examples of additional agents include timolol, befunolol, betaxolol, carteolol, levobunolol, metipranolol, mepindolol, acetazolamide, brinzolamide, diclofenamide, dorzolamide, methazolamide, epinephrine, brimonidine, apraclonidine, ripasudil, netarsudil, pilocarpine, carbachol, and echothiophate iodide.

### Prostaglandin Agonist Combination Therapy

In some embodiments, a Tie-2 activator or HPTPβ inhibitor can be administered in conjunction with one or more compounds that cause agonism of a prostaglandin receptor for the treatment of a condition, for example, an ocular condition described herein. Prostaglandins are compounds that cause agonism of a prostaglandin receptor. These compounds mediate ocular hypotensive activity by stimulating the prostaglandin F2α receptor. Prostaglandins, also known as prostaglandin agonists or prostaglandin analogues, are ocular hypotensive drugs that can be used for the treatment of glaucoma, elevated intraocular pressure, or ocular hypertension. Prostaglandin analogues are structural analogues of prostaglandin that have ocular hypotensive activity. For example, prostaglandins can reduce intraocular pressure in the eye by increasing the uveoscleral outflow or increasing aqueous humor outflow through the trabecular meshwork and Schlemm's canal.

In some embodiments, a compound that causes agonism of a prostaglandin receptor is a prostaglandin F2α analogue. The prostaglandin F receptor (FP) binds to and mediates the biological actions of Prostaglandin F2α (PGF2a). Stimulation of FP receptors located on ciliary muscle and trabecular meshwork cells of the eye can widen drainage channels formed by these cells, known as the uveoscleral pathway. These drainage channels increase the outflow of aqueous humor from the anterior chamber of the eye through Schlemm's canal to outside of the eyeball. The increase in aqueous humor outflow triggered by FP receptor activation reduces intraocular pressure.

In some embodiments, a compound described herein that causes agonism of a prostaglandin receptor is a prodrug of a prostaglandin agonist. The prodrug can be is metabolized to a biologically active form in a subject. In some embodiments, a compound that causes agonism of a prostaglandin receptor is an isopropyl ester prodrug that is hydrolyzed to the biologically active free acid form by esterases in the stroma of the cornea. In some embodiments, a compound that causes agonism of a prostaglandin receptor is a prostamide. Non-limiting examples of prostamides include bimatoprost and prostamide F2a.

In some embodiments, a compound that causes agonism of a prostaglandin receptor is of the formula:

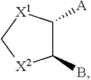

wherein:
A) A is alkyl, alkenyl, alkynyl, allenyl, hydroxyalkyl, or alkoxyalkyl, any of which is substituted or unsubstituted;
   B is alkyl, alkenyl, alkynyl, allenyl, hydroxyalkyl, or alkoxyalkyl, any of which is substituted or unsubstituted;
   X¹ is —C(═O)— or —CH(OH)—; and
   X² is —C(═O)—, or —CH(OH)—; or
B) A is alkyl, alkenyl, alkynyl, allenyl, hydroxyalkyl, or alkoxyalkyl, any of which is substituted or unsubstituted;
   X¹ is —C(═O)— or —CH(OH)—; and
   X² is —CH(G)-, wherein G and B together with the atoms to which G and B are bound form a substituted cycloalkyl, substituted cycloalkenyl, substituted heterocyclylalkyl, or substituted heterocyclylalkenyl; or
C) B is alkyl, alkenyl, alkynyl, allenyl, hydroxyalkyl, or alkoxyalkyl, any of which is substituted or unsubstituted;
   X¹ is —CH(G)-, wherein G and A together with the atoms to which G and A are bound form a substituted cycloalkyl, substituted cycloalkenyl, substituted heterocyclylalkyl, or substituted heterocyclylalkenyl; and
   X² is —C(═O)— or —CH(OH)—,
      or a pharmaceutically acceptable salt thereof.

In some embodiments, a compound that causes agonism of a prostaglandin receptor is of the formula:

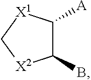

wherein:
A) A is alkyl, alkenyl, alkynyl, allenyl, hydroxyalkyl, or alkoxyalkyl, any of which is substituted or unsubstituted;
   B is alkyl, alkenyl, alkynyl, allenyl, hydroxyalkyl, or alkoxyalkyl, any of which is substituted or unsubstituted;
   X¹ is —C(═O)— or —CH(OH)—; and
   X² is —C(═O)—, or —CH(OH)—; or
B) A is alkyl, alkenyl, alkynyl, allenyl, hydroxyalkyl, or alkoxyalkyl, any of which is substituted or unsubstituted;
   X¹ is —C(═O)— or —CH(OH)—; and
   X² is —CH(G)-, wherein G and B together with the atoms to which G and B are bound form a substituted cycloalkyl, substituted cycloalkenyl, substituted heterocyclylalkyl, or substituted heterocyclylalkenyl; or
C) B is alkyl, alkenyl, alkynyl, allenyl, hydroxyalkyl, or alkoxyalkyl, any of which is substituted or unsubstituted;
   X¹ is —CH(G)-, wherein G and A together with the atoms to which G and A are bound form a substituted cycloalkyl, substituted cycloalkenyl, substituted heterocyclylalkyl, or substituted heterocyclylalkenyl; and
   X² is —C(═O)— or —CH(OH)—.

In some embodiments, a compound that causes agonism of a prostaglandin receptor is of the formula:

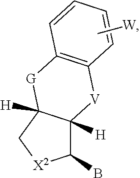

wherein:
B is alkyl, alkenyl, alkynyl, allenyl, hydroxyalkyl, or alkoxyalkyl, any of which is substituted or unsubstituted;

### X² is —C(═O)— or —CH(OH)—;

G is methylene or O;
V is a bond or methylene; and
W is —(O)ₛ(CH₂)ₙC(═O)OR¹, —(CH₂)ₙC(═O)N(R¹)R², —(CH₂)ₙOR¹, or —(O)ₛ(CH₂)ₙR¹, wherein
R¹ and R² are each independently H, C₁-C₆ alkyl, C₁-C₆ cycloalkyl, —SO₂Me, or substituted or unsubstituted aryl;
s is 0 or 1; and
n is 0, 1, 2, 3, 4, or 5.

In some embodiments, B is

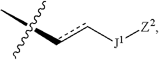

wherein:
each [symbol] indicates a single or double bond;
J¹ is —C(═O)—, —CH₂C(R^{A})(R^{B})—, or —C(R^{A})(R^{B})—, wherein R^{A} and R^{B} are each independently H, hydroxyl, fluoro, chloro, bromo, methyl, or ethyl; and
Z² is —(CH₂)ₙC(═O)OR⁷, —(CH₂)ₙC(═O)N(R⁷)R⁸—(CH₂)ₙOR⁷, or —(CH₂)ₙR⁹, wherein
R⁷ and R⁸ are each independently H, C₁-C₆ alkyl, C₁-C₆ cycloalkyl, —SO₂Me, or substituted or unsubstituted aryl;
R⁹ is H, C₁-C₆ alkyl, C₁-C₆ alkenyl, C₁-C₆ alkynyl, C₁-C₆ cycloalkyl, or substituted or unsubstituted aryl; and
n is 0, 1, 2, 3, 4, or 5.

In some embodiments, Z² is n-butyl, n-pentyl, n-hexyl, n-heptyl, or hex-4-yn-2-yl.

In some embodiments, J¹ is methylene, hydroxymethylene, fluoromethylene, or difluoromethylene.

In some embodiments, W is —(O)ₛ(CH₂)ₙCO₂H.

In some embodiments, a compound that causes agonism of a prostaglandin receptor is of the formula:

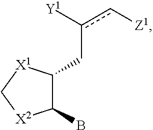

wherein:
A) each [symbol] indicates a single or double bond;
B is alkyl, alkenyl, alkynyl, allenyl, hydroxyalkyl, or alkoxyalkyl, any of which is substituted or unsubstituted;
X¹ is —C(═O)— or —CH(OH)—;
X² is —C(═O)— or —CH(OH)—;
Y¹ is H; and
Z¹ is —(CH₂)ₙC(═O)OR⁴, —(CH₂)ₙC(═O)N(R⁴)R⁵—(CH₂)ₙOR⁴, or —(CH₂)ₙR⁶, wherein
   R⁴ and R⁵ are each independently H, C₁-C₆ alkyl, C₁-C₆ cycloalkyl, —SO₂Me, or substituted or unsubstituted aryl;
   R⁶ is H, C₁-C₆ alkyl, C₁-C₆ alkenyl, C₁-C₆ alkynyl, C₁-C₆ cycloalkyl, or substituted or unsubstituted aryl; and
   n is 0, 1, 2, 3, 4, or 5; or
      B) each [symbol] indicates a single or double bond;
B is alkyl, alkenyl, alkynyl, allenyl, hydroxyalkyl, or alkoxyalkyl, any of which is substituted or unsubstituted;
X¹ is CH-G, wherein G and Y¹ together with the atoms to which G and Y¹ are bound form a substituted cycloalkyl, substituted cycloalkenyl, substituted heterocyclylalkyl, or substituted heterocyclylalkenyl;
X² is —C(═O)— or —CH(OH)—; and
Z¹ is —(CH₂)ₙC(═O)OR⁴, —(CH₂)ₙC(═O)N(R⁴)R⁵—(CH₂)ₙOR⁴, or —(CH₂)ₙR⁶, wherein
   R⁴ and R⁵ are each independently H, C₁-C₆ alkyl, C₁-C₆ cycloalkyl, —SO₂Me, or substituted or unsubstituted aryl;
   R⁶ is H, C₁-C₆ alkyl, C₁-C₆ alkenyl, C₁-C₆ alkynyl, C₁-C₆ cycloalkyl, or substituted or unsubstituted aryl; and
   n is 0, 1, 2, 3, 4, or 5.

In some embodiments, a compound that causes agonism of a prostaglandin receptor is of the formula:

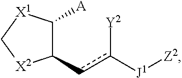

wherein:
A) each [symbol] indicates a single or double bond;
A is alkyl, alkenyl, alkynyl, allenyl, hydroxyalkyl, or alkoxyalkyl, any of which is substituted or unsubstituted;
X¹ is —C(═O)— or —CH(OH)—;
X² is —C(═O)— or —CH(OH)—;
Y² is H;
J¹ is —C(═O)—, —CH₂C(R^{A})(R^{B})—, or —C(R^{A})(R^{B})—, wherein R^{A} and R^{B} are each independently H, hydroxyl, fluoro, chloro, bromo, methyl, or ethyl; and
Z² is —(CH₂)ₙC(═O)OR⁷, —(CH₂)ₙC(═O)N(R⁷)R⁸—(CH₂)ₙOR⁷, or —(CH₂)ₙR⁹, wherein
   R⁷ and R⁸ are each independently H, C₁-C₆ alkyl, C₁-C₆ cycloalkyl, —SO₂Me, or substituted or unsubstituted aryl;
   R⁹H, C₁-C₆ alkyl, C₁-C₆ alkenyl, C₁-C₆ alkynyl, C₁-C₆ cycloalkyl, or substituted or unsubstituted aryl; and
   n is 0, 1, 2, 3, 4, or 5; or
      B) each [symbol] indicates a single or double bond;
A is alkyl, alkenyl, alkynyl, allenyl, hydroxyalkyl, or alkoxyalkyl, any of which is substituted or unsubstituted;
X¹ is —C(═O)— or —CH(OH)—;
X² is —CH(G)-, wherein G and Y² together with the atoms to which G and Y² are bound form a substituted cycloalkyl, substituted cycloalkenyl, substituted heterocyclylalkyl, substituted heterocyclylalkenyl, substituted aryl, or substituted heteroaryl ring;
J¹ is —C(═O)—, —CH₂C(R^{A})(R^{B})—, or —C(R^{A})(R^{B})—, wherein R^{A} and R^{B} are each independently H, hydroxyl, fluoro, chloro, bromo, methyl, or ethyl; and
Z² is —(CH₂)ₙC(═O)OR⁷, —(CH₂)ₙC(═O)N(R⁷)R⁸—(CH₂)ₙOR⁷, or —(CH₂)ₙR⁹, wherein
   R⁷ and R⁸ are each independently H, C₁-C₆ alkyl, C₁-C₆ cycloalkyl, —SO₂Me, or substituted or unsubstituted aryl;
   R⁹H, C₁-C₆ alkyl, C₁-C₆ alkenyl, C₁-C₆ alkynyl, C₁-C₆ cycloalkyl, or substituted or unsubstituted aryl; and
   n is 0, 1, 2, 3, 4, or 5.

In some embodiments, a compound that causes agonism of a prostaglandin receptor is of the formula:

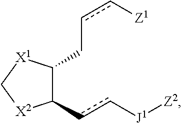

wherein:
each [symbol] independently indicates a single or double bond;

### X¹ is —C(═O)— or —CH(OH)—;

### X² is —C(═O)— or —CH(OH)—;

J¹¹ is —C(═O)—, —CH₂C(R^{A})(R^{B})—, or —C(R^{A})(R^{B})—, wherein R^{A} and R^{B} are each independently H, hydroxyl, fluoro, chloro, bromo, methyl, or ethyl; and
Z¹ and Z² are each independently —(CH₂)ₙC(═O)N(R¹⁰)R¹¹, (CH₂)ₘC(═O)OR¹², —(CH₂)ₒOR¹³, —C(R¹⁴)(R¹⁵)C(R¹⁶)(R¹⁷)R¹⁸, or —(CH₂)ₚR¹⁹, wherein
each R¹⁰, R¹¹, R¹³, R¹⁸, and R¹⁹ is independently H, C₁-C₆ alkyl, C₁-C₆ alkenyl, C₁-C₆ alkynyl, C₁-C₆ cycloalkyl, —SO₂Me, or substituted or unsubstituted aryl;
each R¹⁴, R¹⁵, R⁶, and R¹⁷ is independently H, hydroxyl, fluoro, chloro, bromo, methyl, or ethyl; and
each m, n, o, and p is independently 0, 1, 2, 3, 4, or 5.

In some embodiments, a compound that causes agonism of a prostaglandin receptor is of the formula:

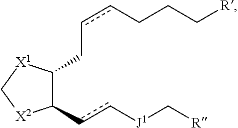

wherein:
each [symbol] independently indicates a single or double bond;

### X¹ is —C(═O)— or —CH(OH)—;

### X² is —C(═O)— or —CH(OH)—;

J¹ is —C(═O)—, —CH₂C(R^{A})(R^{B})—, or —C(R^{A})(R^{B})—, wherein R^{A} and R^{B} are each independently H, hydroxyl, fluoro, chloro, bromo, methyl, or ethyl; and
R′ is —CO₂Me, —CO₂Et, —CO₂i-Pr, —CO₂H, —CO₂NHMe, —CO₂NHEt, or —CO₂NHSO₂Me; and
R″ is —CH₂CH₂Ph, —CH₂CH₂Cy, —OPh, 3-(trifluoromethyl)phenoxy, 3-chlorophenoxy, n-butyl, n-pentyl, or n-hexyl;

In some embodiments, R^{A} is H and R^{B} is hydroxy.

In some embodiments, R^{A} is methyl and R^{B} is hydroxy.

In some embodiments, R^{A} and R^{B} are fluoro.

In some embodiments, a compound that causes agonism of a prostaglandin receptor is:

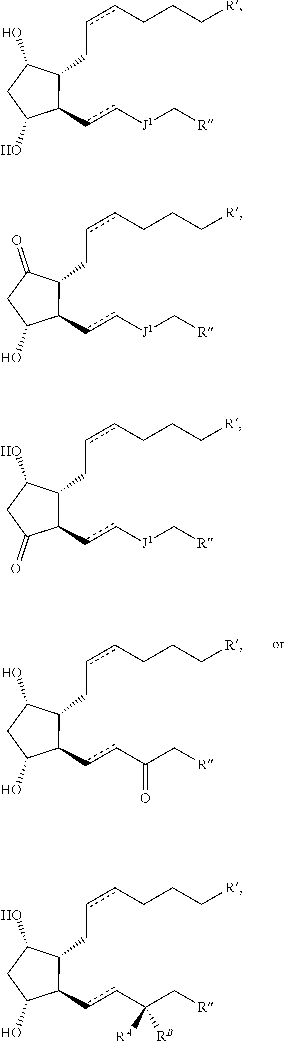

wherein:
each [symbol] independently indicates a single or double bond;
J¹ is —C(═O)—, —CH₂C(R^{A})(R^{B})—, or —C(R^{A})(R^{B})—, wherein R^{A} and R^{B} are each independently H, hydroxyl, fluoro, chloro, bromo, methyl, or ethyl;
R′ is —CO₂Me, —CO₂Et, —CO₂i-Pr, —CO₂H, —CO₂NHMe, —CO₂NHEt, or —CO₂NHSO₂Me; and
R″ is —CH₂CH₂Ph, —CH₂CH₂Cy, —OPh, 3-(trifluoromethyl)phenoxy, 3-chlorophenoxy, n-butyl, n-pentyl, or n-hexyl.

In some embodiments, a compound that causes agonism of a prostaglandin receptor is:

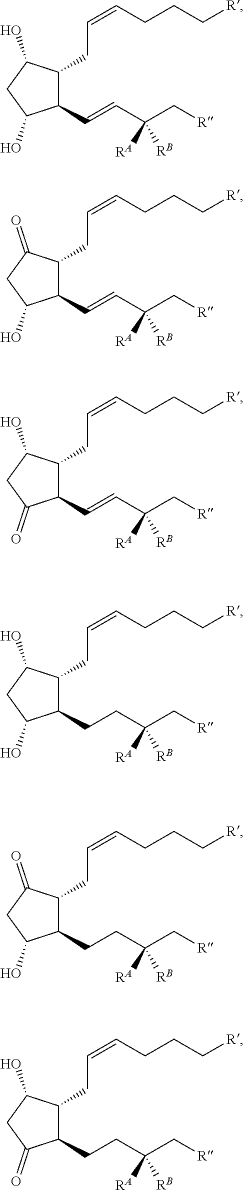

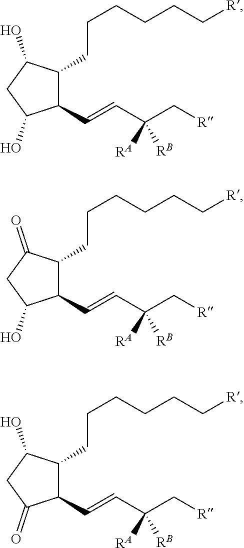

wherein:
R^{A} and R^{B} are each independently H, hydroxyl, fluoro, chloro, bromo, methyl, or ethyl;
R′ is —CO₂Me, —CO₂Et, —CO₂i-Pr, —CO₂H, —CO₂NHMe, —CO₂NHEt, or —CO₂NHSO₂Me; and
R″ is —CH₂CH₂Ph, —CH₂CH₂Cy, —OPh, 3-(trifluoromethyl)phenoxy, 3-chlorophenoxy, n-butyl, n-pentyl, or n-hexyl.

In some embodiments, a compound that causes agonism of a prostaglandin receptor is:

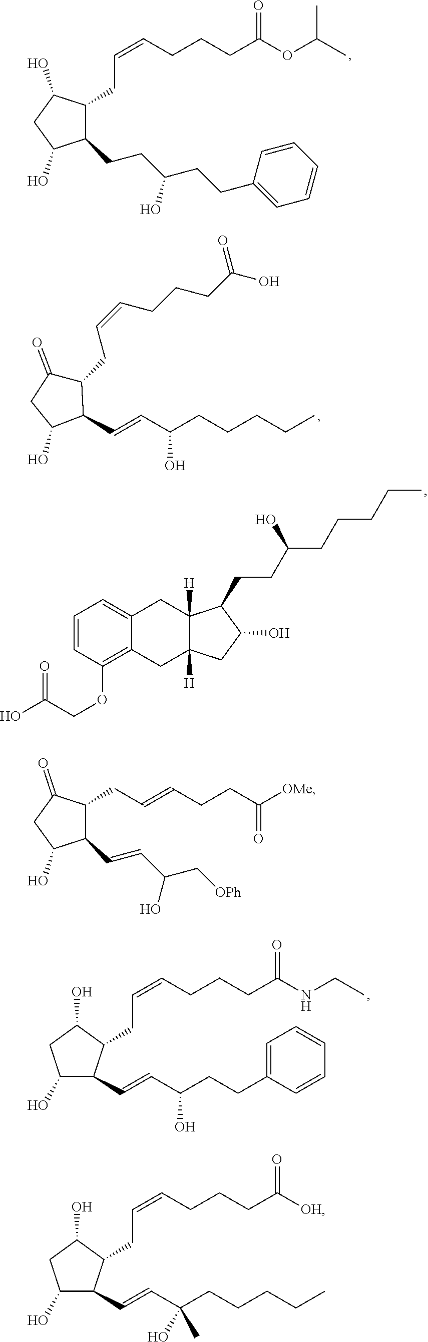

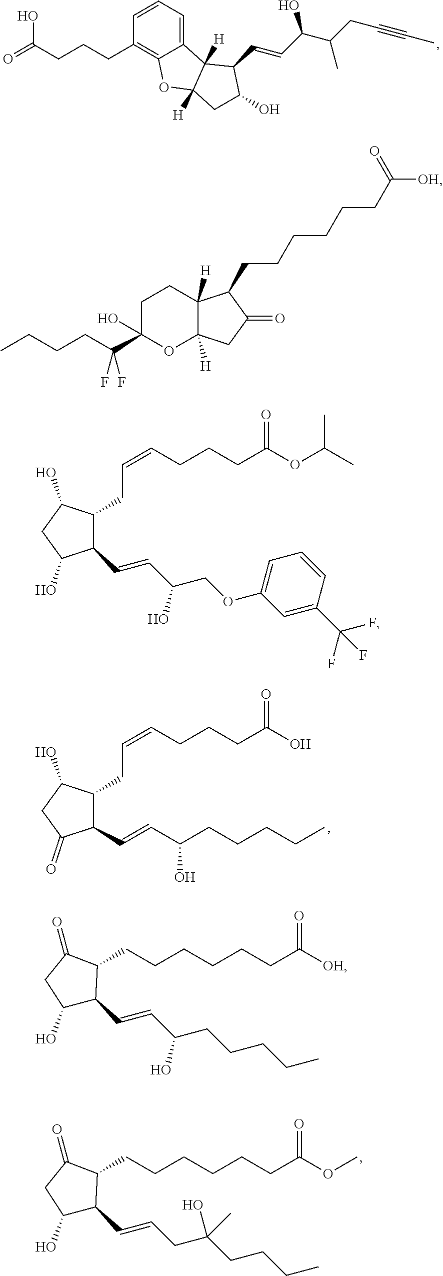

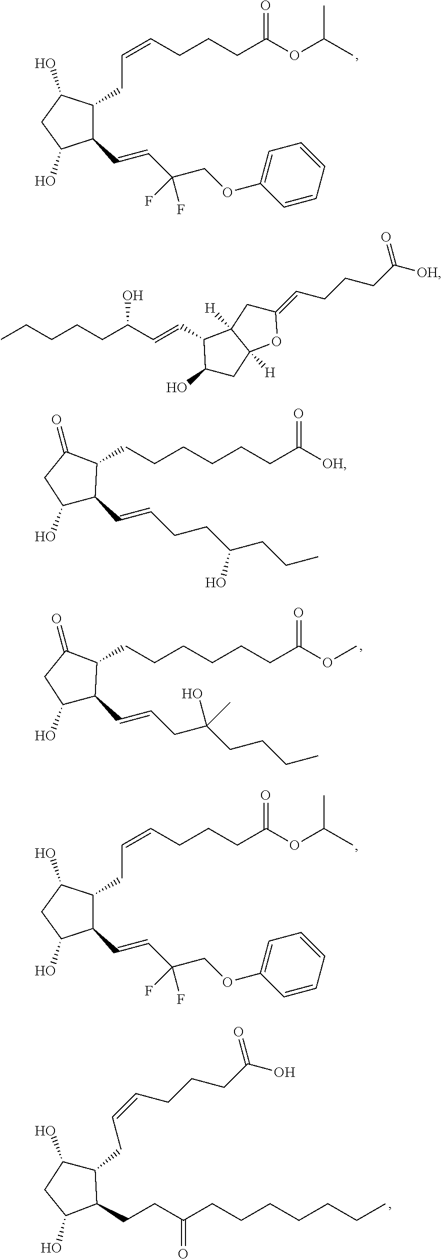

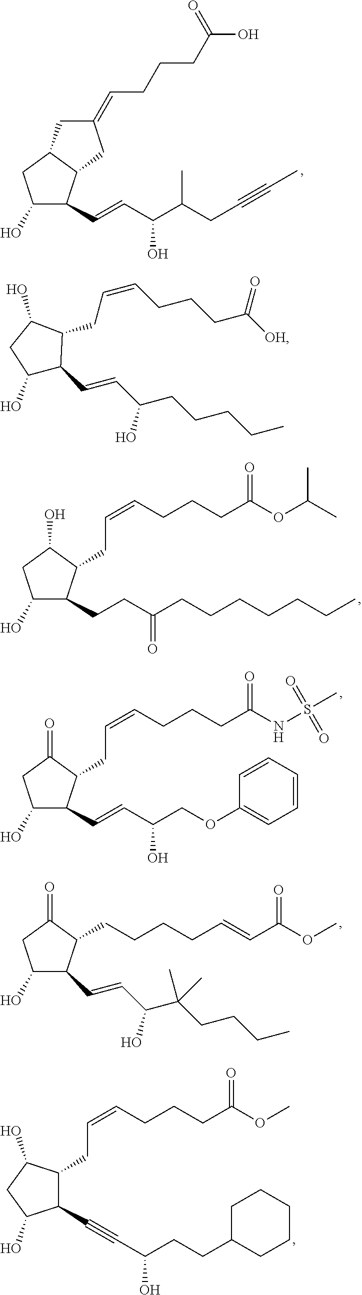

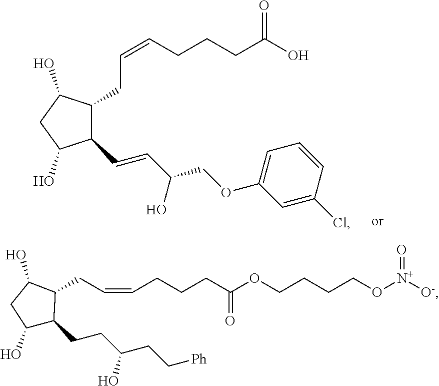

or a pharmaceutically acceptable salt thereof.

In some embodiments, a compound that causes agonism of a prostaglandin receptor is latanoprost, dinoprostone, treprostinil, enprostil, bimatoprost, carboprost, beraprost, lubiprostone, travoprost, prostaglandin D2, prostaglandin E2, dinoprostone, alprostadil, misoprostol, tafluprost, epoprostenol, unoprostone, iloprost, prostaglandin F2a, sulprostone, unoprostone, unoprostone isopropyl, gemeprost, alfaprostol, cloprostenol, latanoprostene bunod, or a pharmaceutically acceptable salt thereof.

Latanoprostene bunod reduces IOP by increasing outflow of aqueous humor through both the trabecular meshwork and uveoscleral routes. After topical ocular administration, latanoprostene bunod is rapidly metabolized in the eye to latanoprost acid (active moiety), an F2α prostaglandin analog, and butanediol mononitrate. The prostaglandin analogue moiety (latanoprost acid) increases uveoscleral outflow, whereas nitric oxide, released by the nitric oxide-donating moiety (butanediol mononitrate), increases outflow through the trabecular meshwork and the Schlemm's canal.

Travoprost reduces IOP by increasing uveoscleral outflow. Travoprost, an isopropyl ester prodrug, is hydrolyzed by esterases in the cornea to its biologically active free acid.

Tafluprost is a fluorinated analog of prostaglandin F2α. Tafluprost, an ester prodrug, is hydrolyzed to its biologically active acid metabolite in the eye. Tafluprost acid can reduce intraocular pressure by increasing uveoscleral outflow.

Unoprostone and unoprostone isopropyl are structural analogues of prostaglandin F2α. Unoprostone and its derivatives can reduce elevated IOP by increasing uveoscleral outflow or increasing aqueous outflow via the conventional trabecular meshwork pathway. Following ocular instillation, unoprostone is hydrolyzed by corneal esterases to its active form, unoprostone free acid. Unoprostone undergoes additional metabolism once inside the eye by iris and ciliary body esterases. Unoprostone can activate potassium (BK) and chloride (CIC-2 type) channels, thereby leading to relaxation of the trabecular meshwork and increased outflow of aqueous humor though the conventional pathway.

Bimatoprost is a synthetic prostamide analog with ocular hypotensive activity. Bimatoprost selectively mimics the effects of naturally occurring substances, prostamides. Bimatoprost can lower IOP by increasing outflow of aqueous humor through both the trabecular meshwork and uveoscleral routes. Bimatoprost is well absorbed through the cornea.

A Tie-2 activator or HPTPβ inhibitor can be co-formulated, co-administered, or administered in conjunction with one or more compounds that cause agonism of a prostaglandin receptor for the treatment of glaucoma, elevated intraocular pressure, or ocular hypertension.

In some embodiments, a Tie-2 activator or HPTPβ inhibitor and a compound that causes agonism of a prostaglandin receptor are present in two different formulations. In some embodiments, the two different formulations are administered simultaneously or concomitantly. In some embodiments, the two different formulations are administered sequentially. In some embodiments, a sub-therapeutic amount of the compound that causes agonism of a prostaglandin receptor is administered. In some embodiments, a therapeutically effective amount of the compound that causes agonism of a prostaglandin receptor is administered.

In some embodiments, a Tie-2 activator or HPTPβ inhibitor and a compound that causes agonism of a prostaglandin receptor are administered sequentially, i.e., the Tie-2 activator or HPTPβ inhibitor is administered either prior to or after the administration of the compound that causes agonism of a prostaglandin receptor. Non-limiting examples of sequential administration include administration of a Tie-2 activator or HPTPβ inhibitor and a compound that causes agonism of a prostaglandin receptor with a time separation of at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 15 minutes, at least 30 minutes, at least 45 minutes, at least 60 minutes, at least 2 hours, at least 3 hours, at least 4 hours, at least 5 hours, at least 6 hours, at least 7 hours, at least 8 hours, at least 9 hours, at least 10 hours, or more. In some embodiments, a Tie-2 activator or HPTPβ inhibitor and a compound that causes agonism of a prostaglandin receptor is sequentially administered with a time separation of about 1 minute, about 5 minutes, about 10 minutes, about 20 minutes, about 30 minutes, about 1 hour, about 6 hours, about 8 hours, or about 12 hours. Either the Tie-2 activator or HPTPβ inhibitor, or the compound that causes agonism of a prostaglandin receptor is administered first. The Tie-2 activator or HPTPβ inhibitor and the compound that causes agonism of a prostaglandin receptor can be contained in separate compositions, which can be contained in the same or different packages.

In some embodiments, the administration of the Tie-2 activator or HPTPβ inhibitor and the compound that causes agonism of a prostaglandin receptor are concurrent, i.e., the administration period of the Tie-2 activator or HPTPβ inhibitor and that of the compound that causes agonism of a prostaglandin receptor overlap with each other. In some embodiments, the administration of the Tie-2 activator or HPTPβ inhibitor and the compound that causes agonism of a prostaglandin receptor are non-concurrent. For example, in some embodiments, the administration of the Tie-2 activator or HPTPβ inhibitor is terminated before the compound that causes agonism of a prostaglandin receptor is administered. In some embodiments, the administration of the compound that causes agonism of a prostaglandin receptor is terminated before the Tie-2 activator or HPTPβ inhibitor is administered. The time period between these two non-concurrent administrations can range from being days apart to being weeks apart.

The dosing frequency of the Tie-2 activator or HPTPβ inhibitor and the compound that causes agonism of a prostaglandin receptor can be adjusted over the course of the treatment, based on the judgment of the administering physician. When administered separately, the Tie-2 activator or HPTPβ inhibitor and the compound that causes agonism of a prostaglandin receptor can be administered at different dosing frequency or intervals. For example, the Tie-2 activator or HPTPβ inhibitor can be administered twice daily, while the compound that causes agonism of a prostaglandin receptor can be administered more or less frequently. Or, the compound that causes agonism of a prostaglandin receptor can be administered once daily, while the Tie-2 activator or HPTPβ inhibitor can be administered more or less frequently. In addition, the Tie-2 activator or HPTPβ inhibitor and the compound that causes agonism of a prostaglandin receptor can be administered using the same route of administration or using different routes of administration.

In some embodiments, the Tie-2 activator or HPTPβ inhibitor and the compound that causes agonism of a prostaglandin receptor are administered within a single pharmaceutical composition. In some embodiments, the pharmaceutical composition further comprises pharmaceutically acceptable diluents or carrier. According to certain embodiments, the Tie-2 activator or HPTPβ inhibitor and the compound that causes agonism of a prostaglandin receptor are administered within different pharmaceutical composition.

A compound disclosed herein can be administered in therapeutically-effective amounts by various forms and routes including, for example, intravenous, intravitreal, intranasal, intratracheal, intrapulmonary, transmucosal, subcutaneous, intramuscular, oral, rectal, aerosol, parenteral, ophthalmic, pulmonary, transdermal, vaginal, otic, nasal, and topical administration. In some embodiments, a compound that causes agonism of a prostaglandin receptor is administered as a topical eye drop.

A compound disclosed herein can be administered at any dose frequency. In some embodiments, a compound that causes agonism of a prostaglandin receptor is administered once daily, twice daily, three times daily, weekly, twice weekly, once every two weeks, or monthly.

In some embodiments, a compound disclosed herein can be present in a composition or dose in an amount at a weight-by-weight, mass-by-mass, weight-by-volume, mass-by-volume, or volume-by-volume percentage of about 0.001% to about 20%. In some embodiments, a compound that causes agonism of a prostaglandin receptor can be present in a composition at a percentage of about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In some embodiments, a compound disclosed herein can be present in a composition or dose in a range of from about 1 μg to about 5 μg, from about 5 μg to about 10 μg, from about 10 μg to about 15 μg, from about 15 μg to about 20 μg, from about 20 μg to about 25 μg, from about 25 μg to about 30 μg, from about 30 μg to about 35 μg, from about 35 μg to about 40 μg, from about 40 μg to about 45 μg, from about 45 μg to about 50 μg, from about 50 μg to about 55 μg, from about 55 μg to about 60 μg, from about 60 μg to about 65 μg, from about 65 μg to about 70 μg, from about 70 μg to about 75 μg, from about 75 μg to about 80 μg, from about 80 μg to about 85 μg, from about 85 μg to about 90 μg, from about 90 μg to about 95 μg, or from about 95 μg to about 100 μg.

In some embodiments, a compound disclosed herein can be present in a composition or dose in an amount of about 0.5 μg, about 1 μg, about 1.5 μg, about 2 μg, about 2.5 μg, about 3 μg, about 3.5 μg, about 4 μg, about 4.5 μg, about 5 μg, about 6 μg, about 7 μg, about 8 μg, about 9 μg, about 10 μg, about 11 μs, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 55 μg, about 60 μg, about 65 μg, about 70 μg, about 75 μg, about 80 μg, about 85 μg, about 90 μg, about 95 μg, about 100 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, or about 1000 μg.

In some embodiments, a compound disclosed herein can be present in a composition or dose in an amount at a weight-by-weight, mass-by-mass, weight-by-volume, mass-by-volume, or volume-by-volume percentage of about 0.001% to about 5%. For example, a compound disclosed herein can be present in a composition or dose in an amount of about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, or about 5%.

In some embodiments, a compound disclosed herein is administered as a topical eye drop, each drop having a volume of about 20 μL, about 25 μL, about 30 μL, about 31 μL, about 32 μL, about 33 μL, about 34 μL, about 35 μL, about 40 μL, about 45 μL, or about 50 μL.

In some embodiments, a Tie-2 activator or HPTPβ inhibitor is administered in conjunction with a compound that causes agonism of a prostaglandin receptor with a separate drop administration of at least 1 minute, at least 2 minutes, at least 3 minutes, at least 4 minutes, at least 5 minutes, at least 6 minutes, at least 7 minutes, at least 8 minutes, at least 9 minutes, at least 10 minutes, at least 15 minutes, at least 30 minutes, at least 45 minutes, at least 60 minutes, at least 2 hours, at least 3 hours, at least 4 hours, at least 5 hours, at least 6 hours, at least 7 hours, at least 8 hours, at least 9 hours, at least 10 hours, or more between administrations of the two therapies.

In some embodiments, a Tie-2 activator or HPTPβ inhibitor administered in conjunction with a compound that causes agonism of a prostaglandin receptor can reduce intraocular pressure by about 1 mmHg to about 20 mmHg, about 1 mmHg to about 10 mmHg, about 1 mmHg to about 5 mmHg, about 2 mmHg to about 8 mmHg, about 3 mmHg to about 6 mmHg, about 5 mmHg to about 10 mmHg, or about 6 mmHg to about 8 mmHg.

In some embodiments, a Tie-2 activator or HPTPβ inhibitor administered in conjunction with a compound that causes agonism of a prostaglandin receptor can reduce intraocular pressure by about 1 mmHg, about 2 mmHg, about 3 mmHg, about 4 mmHg, about 5 mmHg, about 6 mmHg, about 7 mmHg, about 8 mmHg, about 9 mmHg, about 10 mmHg, about 11 mmHg, about 12 mmHg, about 13 mmHg, about 14 mmHg, about 15 mmHg, about 16 mmHg, about 17 mmHg, about 18 mmHg, about 19 mmHg, about 20 mmHg, or greater than 20 mmHg.

In some embodiments, a Tie-2 activator or HPTPβ inhibitor administered in conjunction with a compound that causes agonism of a prostaglandin receptor to a subject can enhance eyelash growth or causes eyelash thickening in the subject.

### Latanoprost Combinations

In some embodiments, the compound that causes agonism of a prostaglandin receptor is latanoprost. In some embodiments, the latanoprost is administered as a 0.005% (50 μg/mL) latanoprost ophthalmic solution. In some embodiments, the ophthalmic solution is a sterile, isotonic, buffered aqueous solution of latanoprost with a pH of approximately 6.7 and an osmolality of approximately 267 mOsmol/kg. Each mL of the latanoprost ophthalmic solution can contain 50 μg of latanoprost. Benzalkonium chloride, 0.02% can be added as a preservative. The inactive ingredients can include sodium chloride, sodium dihydrogen phosphate monohydrate, disodium hydrogen phosphate anhydrous, and water for injection. One drop contains approximately 1.5 μg of latanoprost.

Latanoprost is a prostanoid selective FP receptor agonist that can reduce IOP by increasing the outflow of aqueous humor, for example, by increasing uveoscleral outflow. Elevated IOP is a major risk factor for glaucomatous field loss. The higher the level of IOP, the greater the likelihood of optic nerve damage and visual field loss.

### Pharmacokinetics/Pharmacodynamics

Absorption: Latanoprost is absorbed through the cornea where the isopropyl ester prodrug is hydrolyzed to the acid form to become biologically active. Studies in human indicate that the peak concentration in the aqueous humor can be reached about two hours after topical administration.

Distribution: The distribution volume in humans can be 0.16±0.02 L/kg. The acid of latanoprost can be measured in aqueous humor during the first 4 hours, and in plasma sometimes only during the first hour after local administration.

Metabolism: Latanoprost, an isopropyl ester prodrug, can be hydrolyzed by esterases in the cornea to the biologically active acid. The active acid of latanoprost reaching the systemic circulation can be primarily metabolized by the liver to the 1,2-dinor and 1,2,3,4-tetranor metabolites via fatty acid β-oxidation.

Excretion: The elimination of the acid of latanoprost from human plasma can be rapid (for example, t_{1/2}=17 min) after both intravenous and topical administration. Systemic clearance can be approximately 7 mL/min/kg. Following hepatic β-oxidation, the metabolites can be mainly eliminated via the kidneys. In some embodiments, about 88% and 98% of the administered dose can be recovered in the urine after topical and intravenous dosing, respectively.

### Animal Studies

In some embodiments, latanoprost can induce increased pigmentation of the iris. The mechanism of increased pigmentation can be, for example, stimulation of melanin production in melanocytes of the iris, with no proliferative changes observed. The change in iris color can be permanent.

### Indications and Usage

Latanoprost can be used for the reduction of elevated IOP in patients with open-angle glaucoma or ocular hypertension.

### Clinical Studies

In some embodiments, patients in a controlled study with mean baseline intraocular pressure of 24-25 mmHg who are treated for 6 months in multi-center, randomized, controlled trials can demonstrate 6-8 mmHg reductions in IOP.

### Contraindications

Contraindications can include, for example, hypersensitivity to latanoprost, benzalkonium chloride, or any other ingredients in the latanoprost ophthalmic solution.

### Precautions

Macular edema, including cystoid macular edema, can occur during treatment with latanoprost, for example, in aphakic patients, in pseudophakic patients with a torn posterior lens capsule, or in patients with known risk factors for macular edema. In some embodiments, latanoprost should be used with caution in patients who do not have an intact posterior capsule or who have known risk factors for macular edema.

In some embodiments, contact lenses are removed prior to the administration of latanoprost, and may be reinserted 15 minutes after administration.

### Adverse Reactions

Non-limiting examples of ocular adverse events and ocular signs and symptoms associated with latanoprost ophthalmic solution include blurred vision, burning and stinging, conjunctival hyperemia, foreign body sensation, itching, increased pigmentation of the iris, punctate epithelial keratopathy, dry eye, excessive tearing, eye pain, lid crusting, lid discomfort/pain, lid edema, lid erythema, photophobia, conjunctivitis, diplopia, discharge from the eye, retinal artery embolus, retinal detachment, and vitreous hemorrhage from diabetic retinopathy, upper respiratory tract infection/cold/flu, chest pain/angina pectoris, muscle/joint/back pain, and rash/allergic skin reaction.

Non-limiting examples of adverse events associated with latanoprost ophthalmic solution include: asthma and exacerbation of asthma; corneal edema and erosions; dyspnea; eyelash and vellus hair changes (increased length, thickness, pigmentation, and number); eyelid skin darkening; herpes keratitis; intraocular inflammation (iritis/uveitis); keratitis; macular edema, including cystoid macular edema; misdirected eyelashes sometimes resulting in eye irritation; dizziness, headache, and toxic epidermal necrolysis; periorbital and lid changes resulting in deepening of the eyelid sulcus.

### Dosage and Administration

An example of dosage can be one drop (1.5 μg) in the affected eye(s) once daily in the evening. If one dose is missed, treatment can continue with the next dose as normal.

In some embodiments, the dosage of the latanoprost ophthalmic solution does not exceed once daily.

In some embodiments, reduction of the intraocular pressure starts about 3 to 4 hours after administration and the maximum effect is reached after about 8 to 12 hours.

### How Supplied

The latanoprost ophthalmic solution can be, for example, a clear, isotonic, buffered, preserved colorless solution of latanoprost 0.005% (50 μg/mL). The solution can be supplied as a 2.5 mL solution in a 5 mL clear low density polyethylene bottle with a clear low density polyethylene dropper tip, a turquoise high density polyethylene screw cap, and a tamper-evident clear low density polyethylene overcap.

Storage: The product can be stored protected from light. The product can be stored in unopened bottle(s) under refrigeration at 2° C. to 8° C. (36° F. to 46° F.). During shipment to the patient, the bottle can be maintained at temperatures up to 40° C. (104° F.) for a period not exceeding, for example, 8 days. Once a bottle is opened for use, the bottle can be stored at room temperature up to 25° C. (77° F.) for 6 weeks.

Treatment of Subjects with a Tie-2 Activator

The present disclosure discloses methods for treating a subject afflicted with elevated intraocular pressure with an activator of Tie-2 or an inhibitor of HPTPβ. The subject can be a human. Treatment can include treating a human in a clinical trial. A treatment can comprise administering to a subject a pharmaceutical composition comprising one or more of the activators of Tie-2 described throughout the disclosure. A treatment can comprise administrating to a subject a therapy that promotes the phosphorylation of a Tie-2 molecule.

In some embodiments, the invention provides a Tie-2 activator for use in treatment of elevated intraocular pressure, ocular hypertension, or glaucoma. In some embodiments, the invention provides a Tie-2 activator for use in the manufacture of a medicament for the treatment of elevated intraocular pressure, ocular hypertension, or glaucoma.

In some embodiments, the intraocular pressure or ocular hypertension is caused by a glaucoma. In some embodiments, the glaucoma is open angle glaucoma. In some embodiments, the glaucoma is primary open angle glaucoma. In some embodiments, the glaucoma is inflammatory or neovascular glaucoma.

Non-limiting examples of possible subjects for administration include the following. Subjects can be humans, non-human primates, such as chimpanzees, and other apes and monkey species; farm animals, such as cattle, horses, sheep, goats, and swine; domestic animals, such as rabbits, dogs, and cats; and laboratory animals including rats, mice, and guinea pigs. A subject can be of any age. Subjects can be, for example, elderly adults, adults, adolescents, pre-adolescents, children, toddlers, and infants.

Some conditions can lead to an increase in the levels of Ang-2, altering the ratio of Ang-1/Ang-2 in circulation. In some aspects, a therapy can improve the outcome of a disease state, for example, elevated intraocular pressure or glaucoma, by altering the ratio of Ang-1/Ang-2 in circulation. A therapy can provide an Ang-1/Ang-2 ratio or an Ang-2/Ang-1 ratio of about 1:about 1, about 2:about 1, about 3:about 1, about 4:about 1, about 5:about 1, about 6:about 1, about 7:about 1, about 8:about 1, about 9:about 1, or about 10:about 1.

### EXAMPLES

### Example 1. Compounds with Inhibitory Activity to HPTPβ

Non-limiting examples of the HPTPβ IC₅₀ (μM) activity for illustrative compounds are listed in TABLE 1.

TABLE 1

| | | HPTPβ |
|---|---|---|
| No. | Compound | IC₅₀ μM |
| AA1 | 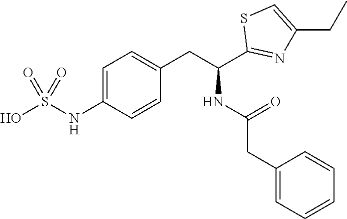 [SMILES: CCc1csc([C@H](Cc2ccc(NS(=O)(=O)O)cc2)NC(=O)Cc2ccccc2)n1] | 0.000157 |
| | (S)-{4-[2-(4-Ethylthiazol-2-yl)-2- | |
| | (phenylacetylamino)ethyl]-phenyl}sulfamic acid | |
| AA2 | 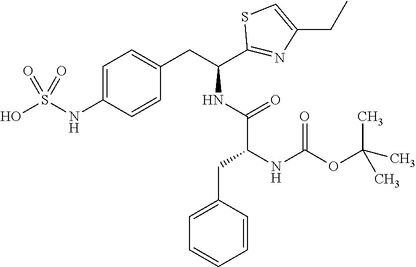 [SMILES: CCc1csc([C@H](Cc2ccc(NS(=O)(=O)O)cc2)NC(=O)[C@@H](Cc2ccccc2)NC(=O)OC(C)(C)C)n1] | 0.004 |
| | 4-{(S)-2-[(R)-2-(tert-butoxycarbonylamino)-3- | |
| | phenylpropanamido]-2-(4-ethylthiazol-2-yl)ethyl}phenylsulfamic acid | |
| AA3 | 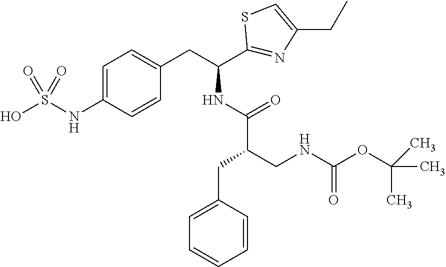 [SMILES: CCc1csc([C@H](Cc2ccc(NS(=O)(=O)O)cc2)NC(=O)[C@H](CNC(=O)OC(C)(C)C)Cc2ccccc2)n1] | 0.031 |
| | {1-[1-(5-Ethylthiazol-2-yl)-(S)-2-(4- | |
| | sulfoaminophenyl)ethyl-carbamoyl]-(S)-2- | |
| | phenylethyl}methyl carbamic acid tert-butyl ester | |
| AA4 | 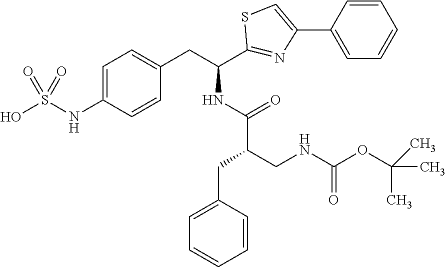 [SMILES: CC(C)(C)OC(=O)NC[C@H](Cc1ccccc1)C(=O)N[C@@H](Cc1ccc(NS(=O)(=O)O)cc1)c1nc(-c2ccccc2)cs1] | <5 × 10⁻⁸ |
| | {1-[1-(5-phenylthiazol-2-yl)-(S)-2- | |
| | (4-sulfoaminophenyl)ethylcarbamoyl]-(S)-2- | |
| | phenylethyl}methylcarbamic acid tert-butyl ester | |
| AA5 | 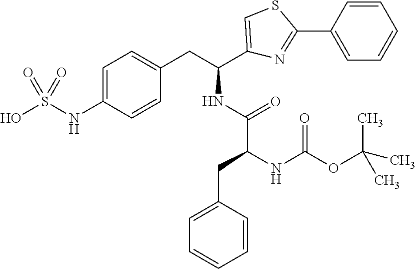 [SMILES: CC(C)(C)OC(=O)N[C@@H](Cc1ccccc1)C(=O)N[C@@H](Cc1ccc(NS(=O)(=O)O)cc1)c1csc(-c2ccccc2)n1] | <5 × 10⁻⁸ |
| | 4-{(S)-2-(S)-2-(tert-Butoxycarbonylamino)-3- | |
| | phenylpropanamido-2-(2-phenylthiazol-4- | |
| | yl)}phenylsulfamic acid | |
| AA6 | 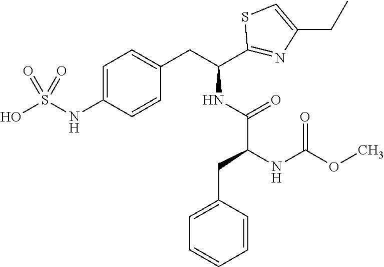 [SMILES: CCc1csc([C@H](Cc2ccc(NS(=O)(=O)O)cc2)NC(=O)[C@H](Cc2ccccc2)NC(=O)OC)n1] | 0.000162 |
| | 4-{(S)-2-(4-Ethylthiazol-2-yl)-2-[(S)-2- | |
| | (methoxycarbonylamino)-3- | |
| | phenylpropanamido]ethyl}phenylsulfamic acid | |
| AA7 | 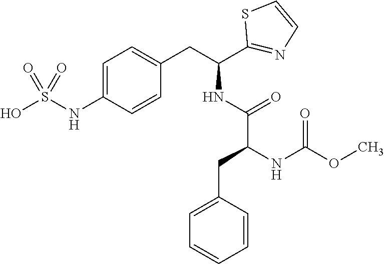 [SMILES: COC(=O)N[C@@H](Cc1ccccc1)C(=O)N[C@@H](Cc1ccc(NS(=O)(=O)O)cc1)c1nccs1] | 0.006 |
| | 4-{(S)-2-[(S)-2-(Methoxycarbonylamino)-3- | |
| | phenylpropanamido]-2-(thiazol-2- | |
| | yl)ethyl}phenylsulfamic acid | |
| AA8 | 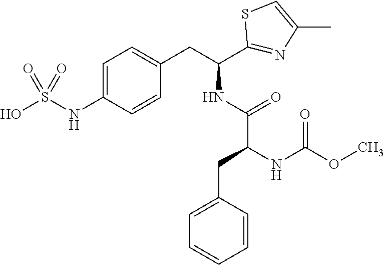 [SMILES: COC(=O)N[C@@H](Cc1ccccc1)C(=O)N[C@@H](Cc1ccc(NS(=O)(=O)O)cc1)c1nc(C)cs1] | 0.001 |
| | 4-{(S)-2-[(S)-2-(Methoxycarbonylamino)-3- | |
| | phenylpropanamido]-2-(4-methylthiazol-2- | |
| | yl)ethyl}phenylsulfamic acid | |
| AA9 | 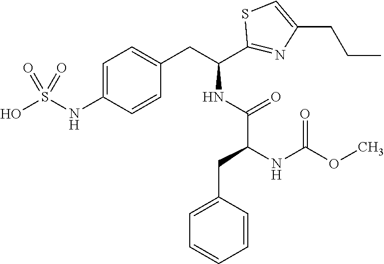 [SMILES: CCCc1csc([C@H](Cc2ccc(NS(=O)(=O)O)cc2)NC(=O)[C@H](Cc2ccccc2)NC(=O)OC)n1] | 0.0001 |
| | 4-{(S)-2-[(S)-2-(Methoxycarbonylamino)-3- | |
| | phenylpropanamido]-2-(4-propylthiazol-2- | |
| | yl)ethyl}phenylsulfamic acid | |
| AA10 | 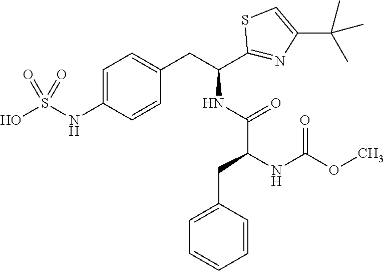 [SMILES: COC(=O)N[C@@H](Cc1ccccc1)C(=O)N[C@@H](Cc1ccc(NS(=O)(=O)O)cc1)c1nc(C(C)(C)C)cs1] | 0.0002 |
| | 4-{(S)-2-(4-tert-Butylthiazol-2-yl)-2-[(S)-2- | |
| | (methoxycarbonylamino)-3- | |
| | phenylpropanamido]ethyl}phenylsulfamic acid | |
| AA11 | 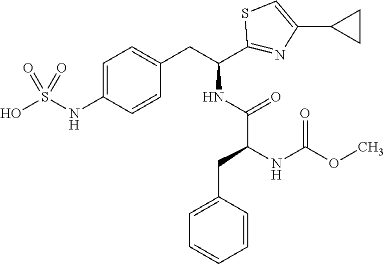 [SMILES: COC(=O)N[C@@H](Cc1ccccc1)C(=O)N[C@@H](Cc1ccc(NS(=O)(=O)O)cc1)c1nc(C2CC2)cs1] | 0.00001 |
| | 4-{(S)-2-(4-Cyclopropylthiazol-2-yl)-2-[(S)-2-(methoxy- | |
| | carbonylamino)-3- | |
| | phenylpropanamido]ethyl}phenylsulfamic acid | |
| AA12 | 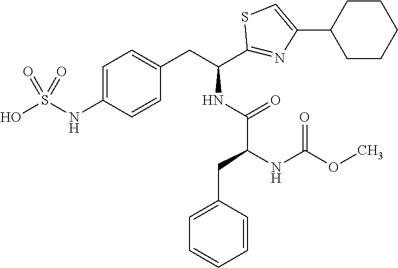 [SMILES: COC(=O)N[C@@H](Cc1ccccc1)C(=O)N[C@@H](Cc1ccc(NS(=O)(=O)O)cc1)c1nc(C2CCCCC2)cs1] | <5 × 10⁻⁸ |
| | 4-{(S)-2-(4-Cyclohexylthiazol-2-yl)-2-[(S)-2- | |
| | (methoxycarbonylamino)-3-phenyl- | |
| | propanamido]ethyl}phenylsulfamic acid | |
| AA13 | 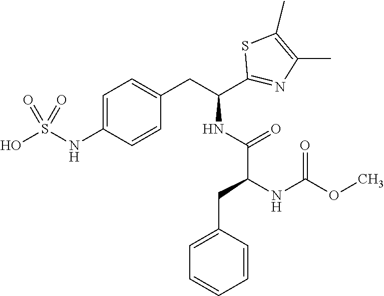 [SMILES: COC(=O)N[C@@H](Cc1ccccc1)C(=O)N[C@@H](Cc1ccc(NS(=O)(=O)O)cc1)c1nc(C)c(C)s1] | 0.001 |
| | 4-{(S)-2-(4,5-Dimethylthiazol-2-yl)-2-[(S)-2- | |
| | (methoxycarbonylamino)-3-phenyl- | |
| | propanamido]ethyl}phenylsulfamic acid | |
| AA14 | 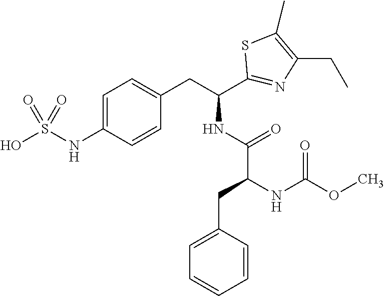 [SMILES: CCc1nc([C@H](Cc2ccc(NS(=O)(=O)O)cc2)NC(=O)[C@H](Cc2ccccc2)NC(=O)OC)sc1C] | 0.0001 |
| | 4-{(S)-2-(4-Ethyl-5-methylthiazol-2-yl)-2-[(S)-2- | |
| | (methoxy-carbonylamino)-3-phenyl- | |
| | propanamido]ethyl}phenylsulfamic acid | |
| AA15 | 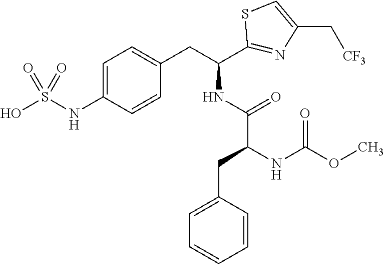 [SMILES: COC(=O)N[C@@H](Cc1ccccc1)C(=O)N[C@@H](Cc1ccc(NS(=O)(=O)O)cc1)c1nc(CC(F)(F)F)cs1] | 0.0003 |
| | 4-{(S)-2-[(S)-2-(Methoxycarbonylamino)-3- | |
| | phenylpropanamido]-2-[4-(2,2,2-trifluoroethyl)thiazol-2- | |
| | yl]ethyl}phenylsulfamic acid | |
| AA16 | 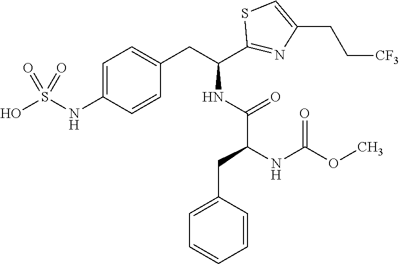 [SMILES: COC(=O)N[C@@H](Cc1ccccc1)C(=O)N[C@@H](Cc1ccc(NS(=O)(=O)O)cc1)c1nc(CCC(F)(F)F)cs1] | 0.00008 |
| | 4-{(S)-2-[(S)-2-(Methoxycarbonylamino)-3- | |
| | phenylpropanam]do)-2-[4-(3,3,3-trifluoropropyl)thiazol- | |
| | 2-yl]et]yl}phenylsulfamic acid | |
| AA17 | 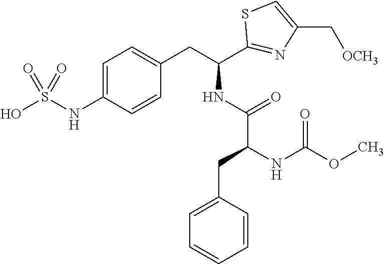 [SMILES: COCc1csc([C@H](Cc2ccc(NS(=O)(=O)O)cc2)NC(=O)[C@H](Cc2ccccc2)NC(=O)OC)n1] | 0.001 |
| | 4-{(S)-2-[(S)-2-(Methoxycarbonylamino)-3- | |
| | phenylpropanamido]-2-[4-(methoxymethyl)thiazol-2- | |
| | yl]ethyl}phenylsulfamic acid | |
| AA18 | 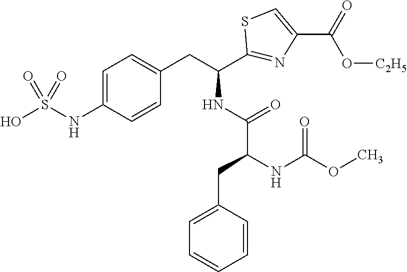 [SMILES: CCOC(=O)c1csc([C@H](Cc2ccc(NS(=O)(=O)O)cc2)NC(=O)[C@H](Cc2ccccc2)NC(=O)OC)n1] | 0.0002 |
| | 4-{(S)-2-(4-(Ethoxycarbonyl)thiazol-2-yl)-2-[(S)-2- | |
| | (methoxy-carbonylamino)-3- | |
| | phenylpropanamido]ethyl}phenylsulfamic acid | |
| AA19 | 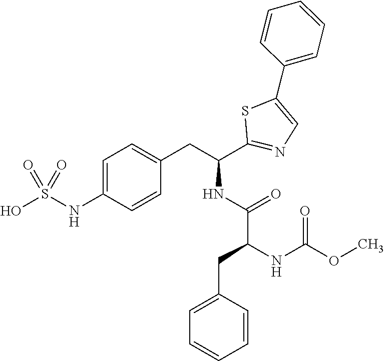 [SMILES: COC(=O)N[C@@H](Cc1ccccc1)C(=O)N[C@@H](Cc1ccc(NS(=O)(=O)O)cc1)c1ncc(-c2ccccc2)s1] | 0.0003 |
| | 4-{(S)-2-[(S)-2-(Methoxycarbonylamino)-3- | |
| | phenylpropanamido]-2-(5-phenylthiazol-2- | |
| | yl)ethyl}phenylsulfamic acid | |
| AA20 | 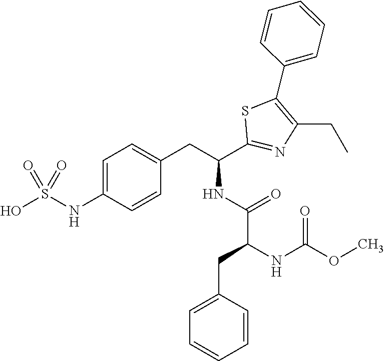 [SMILES: CCc1nc([C@H](Cc2ccc(NS(=O)(=O)O)cc2)NC(=O)[C@H](Cc2ccccc2)NC(=O)OC)sc1-c1ccccc1] | <5 × 10⁻⁸ |
| | 4-{(S)-2-(4-Ethyl-5-phenylthiazol-2-yl)-2-[(S)-2-(methoxy- | |
| | carbonylamino)-3-phenyl-propanamido]ethyl} | |
| | phenylsulfamic acid | |
| AA21 | 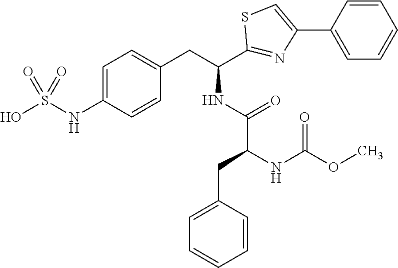 [SMILES: COC(=O)N[C@@H](Cc1ccccc1)C(=O)N[C@@H](Cc1ccc(NS(=O)(=O)O)cc1)c1nc(-c2ccccc2)cs1] | <2 × 10⁻⁶ |
| | 4-{(S)-2-[(S)-2-(Methoxycarbonylamino)-3-phenylpropanamido]- | |
| | 2-(4-phenylthiazol-2-yl)ethyl} | |
| | phenylsulfamic acid | |
| AA22 | 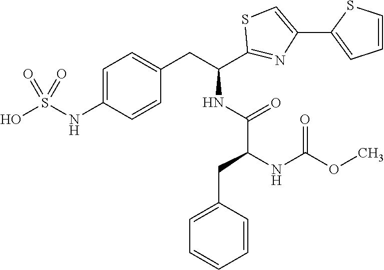 [SMILES: COC(=O)N[C@@H](Cc1ccccc1)C(=O)N[C@@H](Cc1ccc(NS(=O)(=O)O)cc1)c1nc(-c2cccs2)cs1] | <5 × 10⁻⁸ |
| | 4-{(S)-2-[(S)-2-(Methoxycarbonylamino)-3-phenylpropanamido]-2- | |
| | [4-(thiophen-2-yl)thiazol-2-yl]ethyl}phenylsulfamic acid | |
| AA23 | 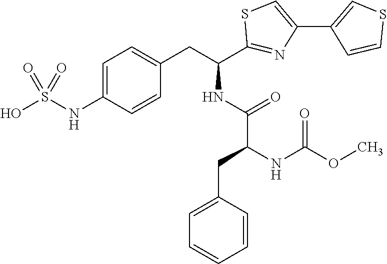 [SMILES: COC(=O)N[C@@H](Cc1ccccc1)C(=O)N[C@@H](Cc1ccc(NS(=O)(=O)O)cc1)c1nc(-c2ccsc2)cs1] | 0.00009 |
| | 4-{(S)-2-[(S)-2-(Methoxycarbonylamino)-3-phenylpropanamido]-2- | |
| | [4-(thiophen-3-yl)thiazol-2-yl]ethyl}phenylsulfamic acid | |
| AA24 | 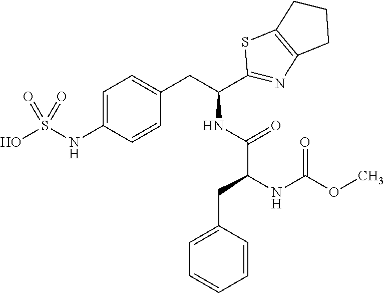 [SMILES: COC(=O)N[C@@H](Cc1ccccc1)C(=O)N[C@@H](Cc1ccc(NS(=O)(=O)O)cc1)c1nc2c(s1)CCC2] | 0.001 |
| | 4-{(S)-2-(5,6-Dihydro-4H-cyclopenta[d]thiazol-2-yl)-2- | |
| | [(S)-2-(methoxycarbonylamino)-3- | |
| | phenylpropanamido]ethyl}phenylsulfamic acid | |
| AA25 | 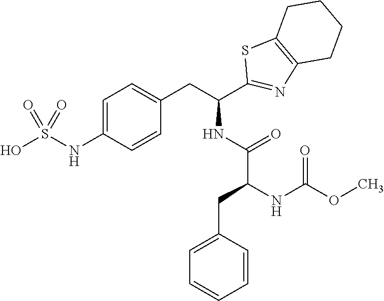 [SMILES: COC(=O)N[C@@H](Cc1ccccc1)C(=O)N[C@@H](Cc1ccc(NS(=O)(=O)O)cc1)c1nc2c(s1)CCCC2] | 0.0004 |
| | 4-{(S)-2-[(S)-2-(Methoxycarbonylamino)-3-phenylpropanamido]-2-(4,5,6,7- | |
| | tetrahydrobenzo[d]thiazol-2-yl)ethyl}phenylsulfamic acid | |
| AA26 | 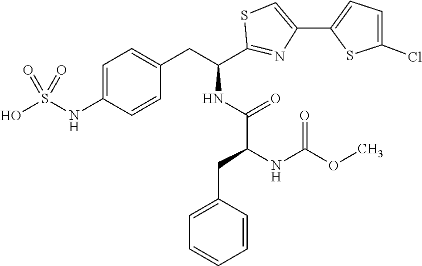 [SMILES: COC(=O)N[C@@H](Cc1ccccc1)C(=O)N[C@@H](Cc1ccc(NS(=O)(=O)O)cc1)c1nc(-c2ccc(Cl)s2)cs1] | <5 × 10⁻⁸ |
| | 4-{(S)-2-[4-(5-Chlorothiophen-2-yl)thiazol-2-yl]-2-[(S)-2- | |
| | (methoxycarbonylamino)-3-phenylpropanamido]ethyl} | |
| | phenyl-sulfamic acid | |
| AA27 | 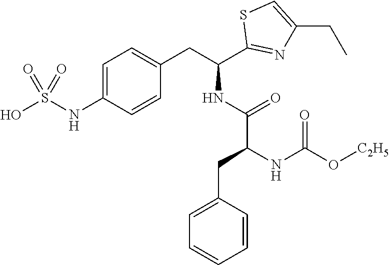 [SMILES: CCOC(=O)N[C@@H](Cc1ccccc1)C(=O)N[C@@H](Cc1ccc(NS(=O)(=O)O)cc1)c1nc(CC)cs1] | 0.00014 |
| | 4-{(S)-2-[(S)-2-(Ethoxycarbonylamino)-3-phenylpropanamido]- | |
| | 2-(4-ethylthiazol-2-yl)ethyl}phenylsulfamic acid | |
| AA28 | 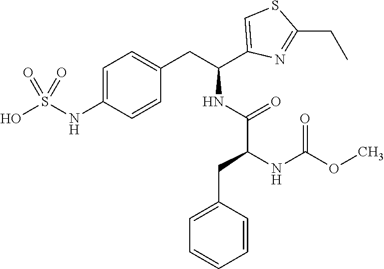 [SMILES: CCc1nc([C@H](Cc2ccc(NS(=O)(=O)O)cc2)NC(=O)[C@H](Cc2ccccc2)NC(=O)OC)cs1] | 0.0001 |
| | 4-{(S)-2-[(S)-2-(Methoxycarbonylamino)-3- | |
| | phenylpropanamido]-2-(2-ethylthiazol-4-yl) | |
| | ethyl}phenylsulfamic acid | |
| AA29 | 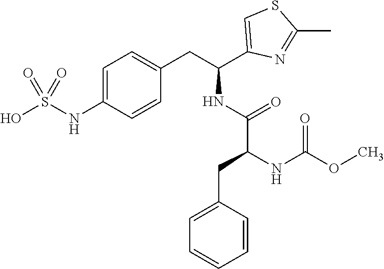 [SMILES: COC(=O)N[C@@H](Cc1ccccc1)C(=O)N[C@@H](Cc1ccc(NS(=O)(=O)O)cc1)c1csc(C)n1] | 0.001 |
| | 4-{(S)-2-[(S)-2-(Methoxycarbonylamino)-3-phenylpropanamido]-2- | |
| | (2-methylthiazol-4-yl)ethyl}phenylsulfamic acid | |
| AA30 | 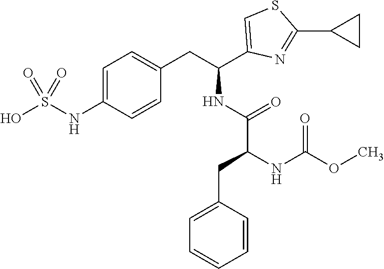 [SMILES: COC(=O)N[C@@H](Cc1ccccc1)C(=O)N[C@@H](Cc1ccc(NS(=O)(=O)O)cc1)c1csc(C2CC2)n1] | 0.0002 |
| | 4-{(S)-2-(2-Cyclopropylthiazol-4-yl)-2-[(S)-2-(methoxy-carbonylamino)-3- | |
| | phenylpropanamido]ethyl}phenylsulfamic acid | |
| AA31 | 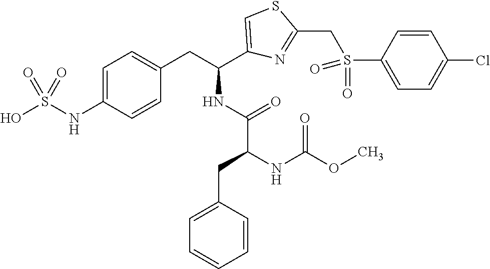 [SMILES: COC(=O)N[C@@H](Cc1ccccc1)C(=O)N[C@@H](Cc1ccc(NS(=O)(=O)O)cc1)c1csc(CS(=O)(=O)c2ccc(Cl)cc2)n1] | 0.00008 |
| | 4-{(S)-2-{2-[(4-Chlorophenylsulfonyl)methyl]thiazol-4-yl}-2-[(S)- | |
| | 2-(methoxycarbonylamino)-3-phenylpropanamido] | |
| | ethyl}phenylsulfamic acid | |
| AA32 | 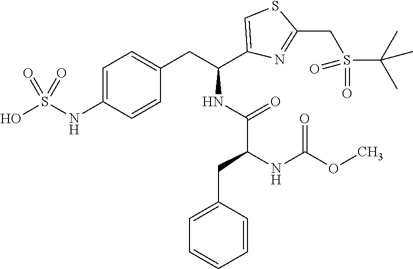 [SMILES: COC(=O)N[C@@H](Cc1ccccc1)C(=O)N[C@@H](Cc1ccc(NS(=O)(=O)O)cc1)c1csc(CS(=O)(=O)C(C)(C)C)n1] | 0.002 |
| | 4-{(S)-2-[2-(tert-Butylsulfonylmethyl)thiazol-4- | |
| | yl]-2-[(S)-2-(methoxycarbonylamino)-3- | |
| | phenylpropanamido]ethyl}phenylsulfamic acid | |
| AA33 | 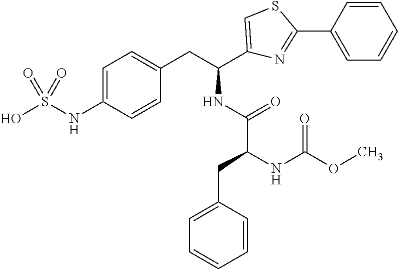 [SMILES: COC(=O)N[C@@H](Cc1ccccc1)C(=O)N[C@@H](Cc1ccc(NS(=O)(=O)O)cc1)c1csc(-c2ccccc2)n1] | 7 × 10⁻⁷ |
| | 4-{(S)-2-[(S)-2-(Methoxycarbonylamino)-3-phenylpropionamido]- | |
| | 2-(2-phenylthiazole-4-yl)ethyl}phenylsulfamic acid | |
| AA34 | 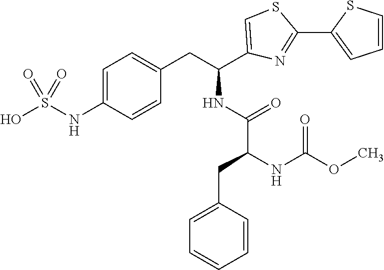 [SMILES: COC(=O)N[C@@H](Cc1ccccc1)C(=O)N[C@@H](Cc1ccc(NS(=O)(=O)O)cc1)c1csc(-c2cccs2)n1] | 5 × 10⁻⁸ |
| | 4-{(S)-2-[(S)-2-(Methoxycarbonylamino)-3-phenylpropanamido]- | |
| | 2-[2-(thiophen-2-yl)thiazol-4-yl]ethyl}phenylsulfamic acid | |
| AA35 | 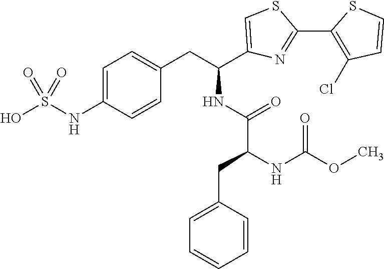 [SMILES: COC(=O)N[C@@H](Cc1ccccc1)C(=O)N[C@@H](Cc1ccc(NS(=O)(=O)O)cc1)c1csc(-c2sccc2Cl)n1] | <5 × 10⁻⁸ |
| | 4-{(S)-2-[2-(3-Chlorothiophen-2-yl)thiazol- | |
| | 4-yl]-2-[(5)-2-(methoxycarbonylamino)-3- | |
| | phenylpropanamido]ethyl}phenylsulfamic acid | |
| AA36 | 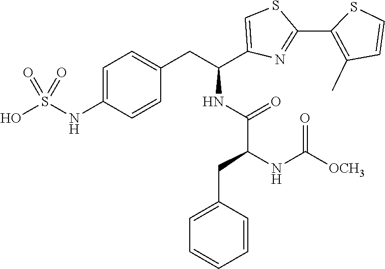 [SMILES: COC(=O)N[C@@H](Cc1ccccc1)C(=O)N[C@@H](Cc1ccc(NS(=O)(=O)O)cc1)c1csc(-c2sccc2C)n1] | <5 × 10⁻⁸ |
| | 4-{(S)-2-[(S)-2-(Methoxycarbonylamino)-3-phenylpropanamido]- | |
| | 2-[2-(3-methylthiophen-2-yl)thiazol-4-yl] | |
| | ethyl}phenylsulfamic acid | |
| AA37 | 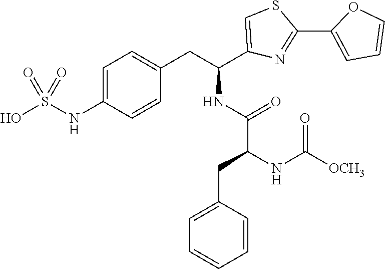 [SMILES: COC(=O)N[C@@H](Cc1ccccc1)C(=O)N[C@@H](Cc1ccc(NS(=O)(=O)O)cc1)c1csc(-c2ccco2)n1] | 0.0004 |
| | 4-{[(S)-2-(2-(Furan-2-yl)thiazol-4)yl]-2-[(S)-2-(methoxy- | |
| | carbonylamino)-3- | |
| | phenylpropanamido]ethyl}phenylsulfamic acid | |
| AA38 | 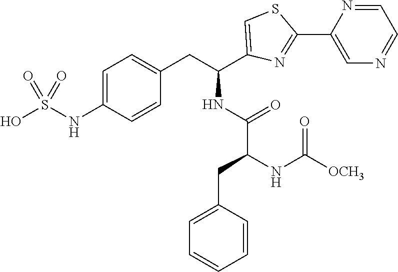 [SMILES: COC(=O)N[C@@H](Cc1ccccc1)C(=O)N[C@@H](Cc1ccc(NS(=O)(=O)O)cc1)c1csc(-c2cnccn2)n1] | 0.003 |
| | 4-{(S)-2-[(S)-2-(Methoxycarbonylamino)-3-phenylpropanamido]-2- | |
| | [2-(pyrazin-2-yl)thiazol-4-yl]ethyl}phenylsulfamic acid | |
| AA39 | 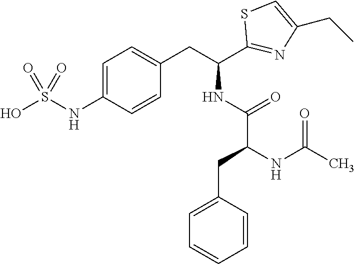 [SMILES: CCc1csc([C@H](Cc2ccc(NS(=O)(=O)O)cc2)NC(=O)[C@H](Cc2ccccc2)NC(C)=O)n1] | 0.001 |
| | 4-[(S)-2-((S)-2-Acetamido-3-phenylpropanamido)-2-(4-ethylthiazol- | |
| | 2-yl)ethyl]phenylsulfamic acid | |
| AA40 | 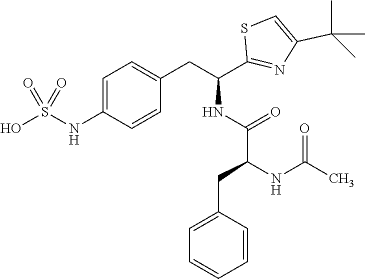 [SMILES: CC(=O)N[C@@H](Cc1ccccc1)C(=O)N[C@@H](Cc1ccc(NS(=O)(=O)O)cc1)c1nc(C(C)(C)C)cs1] | 0.0003 |
| | 4-[(S)-2-((S)-2-Acetamido-3-phenylpropanamido)-2-(4-tert-butylthiazol- | |
| | 2-yl)ethyl]phenylsulfamic acid | |
| AA41 | 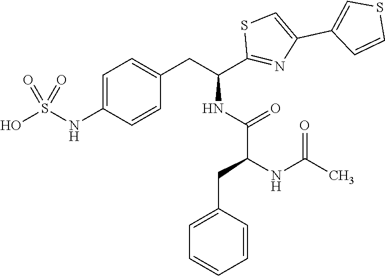 [SMILES: CC(=O)N[C@@H](Cc1ccccc1)C(=O)N[C@@H](Cc1ccc(NS(=O)(=O)O)cc1)c1nc(-c2ccsc2)cs1] | 0.00024 |
| | 4-{(S)-2-((S)-2-Acetamido-3-phenylpropanamido)-2-[4- | |
| | (thiophen-3-yl)thiazol-2-yl]ethyl}phenylsulfamic acid | |
| AA42 | 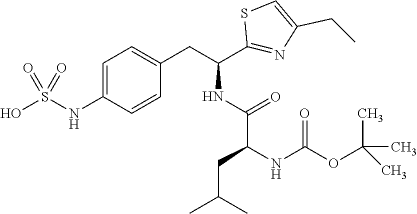 [SMILES: CCc1csc([C@H](Cc2ccc(NS(=O)(=O)O)cc2)NC(=O)[C@H](CC(C)C)NC(=O)OC(C)(C)C)n1] | 0.006 |
| | 4-{(S)-2-[(S)-2-(tert-Butoxycarbonylamino)-3-methylbutanamido]- | |
| | 2-(4-ethylthiazol-2-yl)ethyl}phenylsulfamic acid | |
| AA43 | 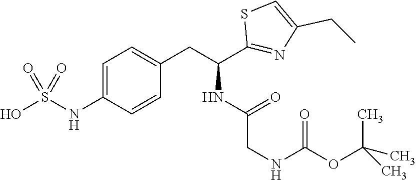 [SMILES: CCc1csc([C@H](Cc2ccc(NS(=O)(=O)O)cc2)NC(=O)CNC(=O)OC(C)(C)C)n1] | 0.028 |
| | (S)-4-{2-[2-(tert-Butoxycarbonylamino)acetamido]-2-(4- | |
| | ethylthiazol-2-yl)ethyl}phenylsulfamic acid | |
| AA44 | 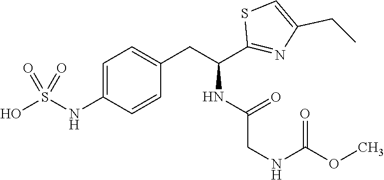 [SMILES: CCc1csc([C@H](Cc2ccc(NS(=O)(=O)O)cc2)NC(=O)CNC(=O)OC)n1] | 0.020 |
| | (S)-4-{2-(4-Ethylthiazol-2-yl)-2-[2- | |
| | (methoxycarbonylamino)acetamido]ethyl}phenylsulfamic | |
| | acid | |
| AA45 | 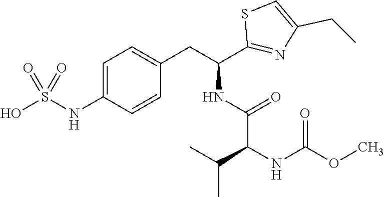 [SMILES: CCc1csc([C@H](Cc2ccc(NS(=O)(=O)O)cc2)NC(=O)[C@@H](NC(=O)OC)C(C)C)n1] | 0.003 |
| | 4-{(S)-2-(4-Ethylthiazol-2-yl)-2-[(S)-2-(methoxycarbonylamino)- | |
| | 3-methylbutanamido]-ethyl}phenylsulfamic acid | |
| AA46 | 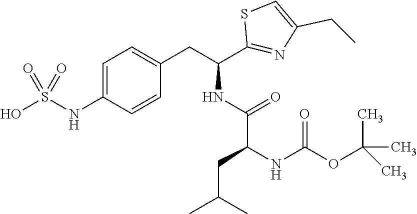 [SMILES: CCc1csc([C@H](Cc2ccc(NS(=O)(=O)O)cc2)NC(=O)[C@H](CC(C)C)NC(=O)OC(C)(C)C)n1] | 0.001 |
| | 4-{(S)-2-[(S)-2-(tert-Butoxycarbonylamino)-4-methylpentanamido]- | |
| | 2-(4-ethylthiazol-2-yl)ethyl}phenylsulfamic acid | |
| AA47 | 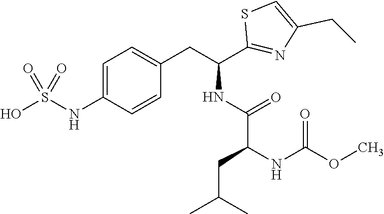 [SMILES: CCc1csc([C@H](Cc2ccc(NS(=O)(=O)O)cc2)NC(=O)[C@H](CC(C)C)NC(=O)OC)n1] | 0.0003 |
| | 4-{(S)-2-(4-Ethylthiazol-2-yl)-2-[(S)-2-(methoxycarbonylamino)- | |
| | 4-methylpentanamido]ethyl}phenylsulfamic acid | |
| AA48 | 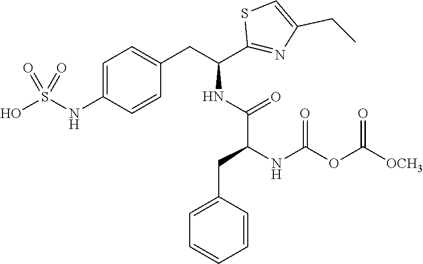 [SMILES: CCc1csc([C@H](Cc2ccc(NS(=O)(=O)O)cc2)NC(=O)[C@H](Cc2ccccc2)NC(=O)OC(=O)OC)n1] | 0.0003 |
| | 4-((S)-2-(4-Ethylthiazol-2-yl)-2-{(S)-2-[2-(methoxycarbonylamino)- | |
| | acetamido]-3-phenylpropanamido}ethyl) | |
| | phenylsulfamic acid | |
| AA49 | 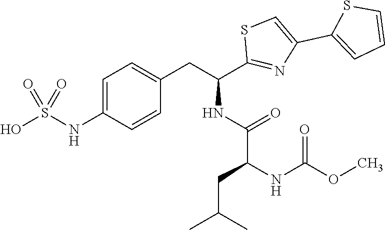 [SMILES: COC(=O)N[C@@H](CC(C)C)C(=O)N[C@@H](Cc1ccc(NS(=O)(=O)O)cc1)c1nc(-c2cccs2)cs1] | <5 × 10⁻⁸ |
| | 4-{(S)-2-[(S)-2-(Methoxycarbonylamino)-4-methylpentanamido]- | |
| | 2-[2-(thiophen-2-yl)thiazol-4-yl]ethyl}phenylsulfamic acid | |
| AA50 | 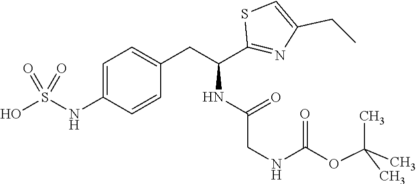 [SMILES: CCc1csc([C@H](Cc2ccc(NS(=O)(=O)O)cc2)NC(=O)CNC(=O)OC(C)(C)C)n1] | 0.028 |
| | (S)-4-{2-[2-(tert-Butoxycarbonylamino)acetamido]-2-(4-ethylthiazol- | |
| | 2-yl)ethyl}-phenylsulfamic acid | |
| AA51 | 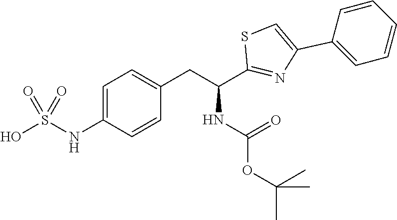 [SMILES: CC(C)(C)OC(=O)N[C@@H](Cc1ccc(NS(=O)(=O)O)cc1)c1nc(-c2ccccc2)cs1] | 0.049 |
| | [1-(S)-(Phenylthiazol-2-yl)-2-(4-sulfoaminophenyl)ethyl]- | |
| | carbamic acid tert-butyl ester | |
| AA52 | 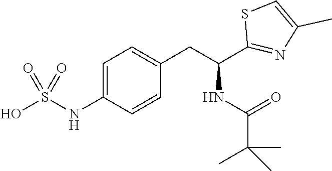 [SMILES: Cc1csc([C@H](Cc2ccc(NS(=O)(=O)O)cc2)NC(=O)C(C)(C)C)n1] | 0.112 |
| | (S)-4-(2-(4-Methylthiazol-2-yl)-2-pivalamidoethyl)phenyl- | |
| | sulfamic acid | |
| AA53 | 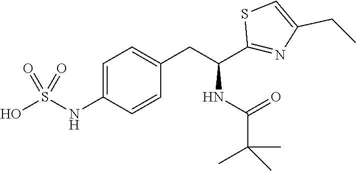 [SMILES: CCc1csc([C@H](Cc2ccc(NS(=O)(=O)O)cc2)NC(=O)C(C)(C)C)n1] | 0.085 |
| | (S)-4-(2-(4-Ethylthiazol-2-yl)-2-pivalamidoethyl)phenyl- | |
| | sulfamic acid | |
| AA54 | 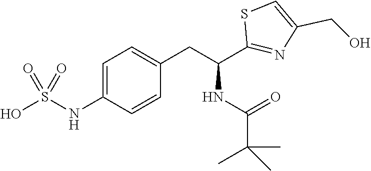 [SMILES: CC(C)(C)C(=O)N[C@@H](Cc1ccc(NS(=O)(=O)O)cc1)c1nc(CO)cs1] | 0.266 |
| | (S)-4-{2-[4-(hydroxymethyl)thiazol-2-yl]-2-pivalamidoethyl} | |
| | phenyl-sulfamic acid | |
| AA55 | 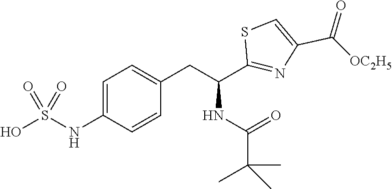 [SMILES: CCOC(=O)c1csc([C@H](Cc2ccc(NS(=O)(=O)O)cc2)NC(=O)C(C)(C)C)n1] | 0.584 |
| | (S)-4-{[2-(4-Ethoxycarbonyl)thiazol-2-yl]-2-pivalamidoethyl} | |
| | phenylsulfamic acid | |
| AA56 | 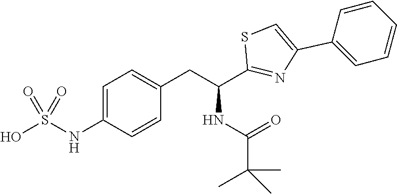 [SMILES: CC(C)(C)C(=O)N[C@@H](Cc1ccc(NS(=O)(=O)O)cc1)c1nc(-c2ccccc2)cs1] | 0.042 |
| | (S)-4-(2-(4-Phenylthiazol-2-yl)-2-pivalamidoethyl) | |
| | phenylsulfamic acid | |
| AA57 | 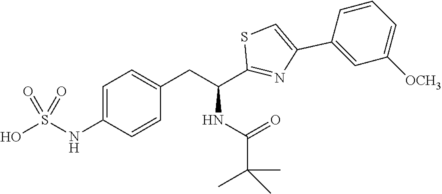 [SMILES: COc1cccc(-c2csc([C@H](Cc3ccc(NS(=O)(=O)O)cc3)NC(=O)C(C)(C)C)n2)c1] | 0.110 |
| | 4-((S)-2-(4-(3-Methoxyphenyl)thiazol-2-yl)-2- | |
| | pivalamidoethyl)phenylsulfamic acid | |
| AA58 | 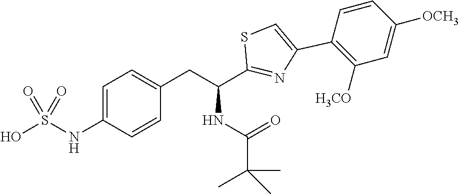 [SMILES: COc1ccc(-c2csc([C@H](Cc3ccc(NS(=O)(=O)O)cc3)NC(=O)C(C)(C)C)n2)c(OC)c1] | 0.086 |
| | 4-((S)-2-(4-(2,4-Dimethoxyphenyl)thiazol-2-yl)-2- | |
| | pivalamidoethyl)phenyl-sulfamic acid | |
| AA59 | 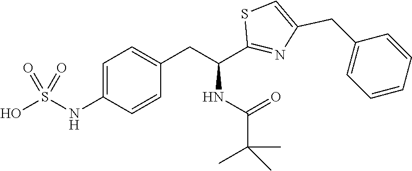 [SMILES: CC(C)(C)C(=O)N[C@@H](Cc1ccc(NS(=O)(=O)O)cc1)c1nc(Cc2ccccc2)cs1] | 0.113 |
| | (S)-4-(2-(4-Benzylthiazol-2-yl)-2-pivalamidoethyl) | |
| | phenylsulfamic acid | |
| AA60 | 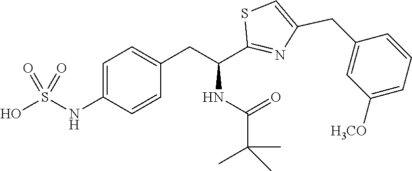 [SMILES: COc1cccc(Cc2csc([C@H](Cc3ccc(NS(=O)(=O)O)cc3)NC(=O)C(C)(C)C)n2)c1] | 0.132 |
| | (S)-4-(2-(4-(3-Methoxybenzyl)thiazol-2-yl)-2-pivalamidoethyl) | |
| | phenylsulfamic acid | |
| AA61 | 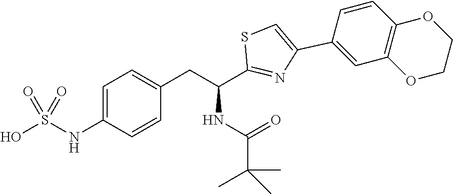 [SMILES: CC(C)(C)C(=O)N[C@@H](Cc1ccc(NS(=O)(=O)O)cc1)c1nc(-c2ccc3c(c2)OCCO3)cs1] | 0.138 |
| | 4-((S)-2-(4-(2,3-Dihydrobenzo[b][1,4]dioxin-6-yl)thiazol-2-yl)- | |
| | 2-pivalamidoethyl)phenylsulfamic acid | |
| AA62 | 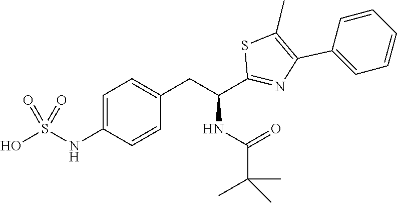 [SMILES: Cc1sc([C@H](Cc2ccc(NS(=O)(=O)O)cc2)NC(=O)C(C)(C)C)nc1-c1ccccc1] | 0.098 |
| | (S)-4-(2-(5-Methyl-4-phenylthiazol-2-yl)-2-pivalamidoethyl) | |
| | phenylsulfamic acid | |
| AA63 | 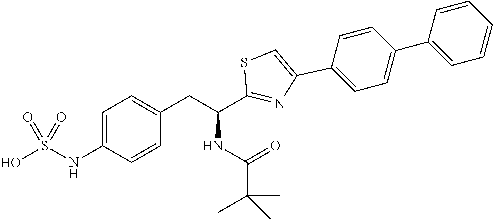 [SMILES: CC(C)(C)C(=O)N[C@@H](Cc1ccc(NS(=O)(=O)O)cc1)c1nc(-c2ccc(-c3ccccc3)cc2)cs1] | 0.381 |
| | (S)-4-(2-(4-(Biphen-4-yl)thiazol-2-yl)-2-pivalamidoethyl) | |
| | phenylsulfamic acid | |
| AA64 | 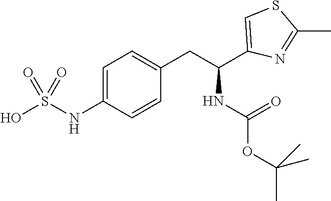 [SMILES: Cc1nc([C@H](Cc2ccc(NS(=O)(=O)O)cc2)NC(=O)OC(C)(C)C)cs1] | 0.033 |
| | (S)-4-(2-tert-Butoxycarbonylamino)-2-(2-methylthiazol-4-yl) | |
| | ethyl)phenylsulfamic acid | |
| AA65 | 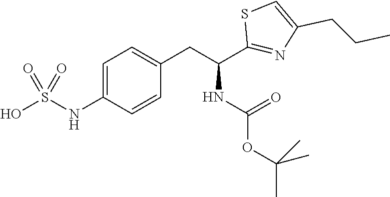 [SMILES: CCCc1csc([C@H](Cc2ccc(NS(=O)(=O)O)cc2)NC(=O)OC(C)(C)C)n1] | 0.04 |
| | (S)-4-(2-(tert-Butoxycarbonylamino)-2-(4-propylthiazol-2-yl) | |
| | ethyl)phenyl sulfamic acid | |
| AA66 | 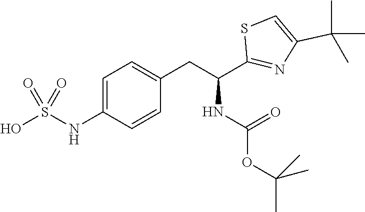 [SMILES: CC(C)(C)OC(=O)N[C@@H](Cc1ccc(NS(=O)(=O)O)cc1)c1nc(C(C)(C)C)cs1] | 0.027 |
| | (S)-4-(2-(tert-Butoxycarbonylamino)-2-(4-tert-butylthiazol-2-yl) | |
| | ethyl)phenyl sulfamic acid | |
| AA67 | 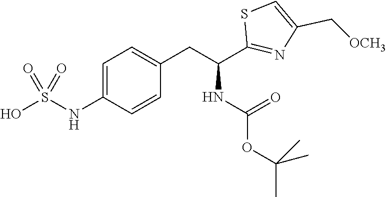 [SMILES: COCc1csc([C@H](Cc2ccc(NS(=O)(=O)O)cc2)NC(=O)OC(C)(C)C)n1] | 0.18 |
| | (S)-4-(2-(tert-Butoxycarbonylamino)-2-(4-(methoxymethyl)thiazol-2- | |
| | yl)ethyl)-phenyl sulfamic acid | |
| AA68 | 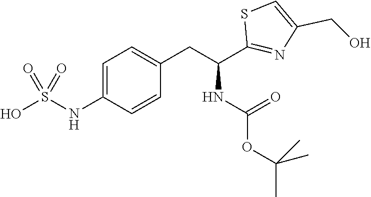 [SMILES: CC(C)(C)OC(=O)N[C@@H](Cc1ccc(NS(=O)(=O)O)cc1)c1nc(CO)cs1] | 0.644 |
| | (S)-4-(2-(tert-Butoxycarbonylamino)-2-(4-(hydroxymethyl)thiazol-2- | |
| | yl)ethyl)phenylsulfamic acid | |
| AA69 | 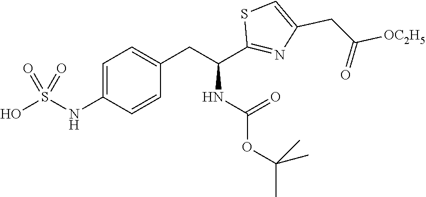 [SMILES: CCOC(=O)Cc1csc([C@H](Cc2ccc(NS(=O)(=O)O)cc2)NC(=O)OC(C)(C)C)n1] | 0.167 |
| | (S)-4-(2-tert-Butoxycarbonylamino)-2-(4-(2-ethoxy-2-oxoethyl) | |
| | thiazol-2-yl)ethyl)phenylsulfamic acid | |
| AA70 | 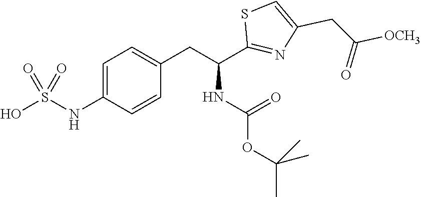 [SMILES: COC(=O)Cc1csc([C@H](Cc2ccc(NS(=O)(=O)O)cc2)NC(=O)OC(C)(C)C)n1] | 0.132 |
| | (S)-4-(2-(tert-Butoxycarbonyl)-2-(4-(2-(2-methoxy-2-oxoyethyl amino)-2- | |
| | oxoethyl)thiazole-2-yl)ethyl)phenylsulfamic acid | |
| AA71 | 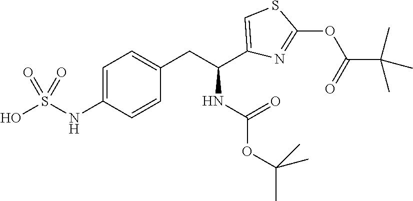 [SMILES: CC(C)(C)OC(=O)N[C@@H](Cc1ccc(NS(=O)(=O)O)cc1)c1csc(OC(=O)C(C)(C)C)n1] | 0.555 |
| | (S)-4-(2-(tert-Butoxycarbonylamino)-2-(2-pivalamidothiazol-4-yl) | |
| | ethyl)phenylsulfamic acid | |
| AA72 | 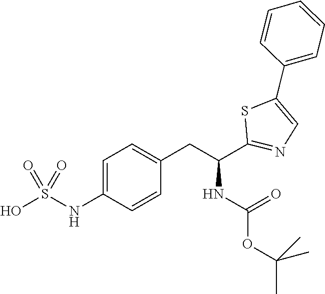 [SMILES: CC(C)(C)OC(=O)N[C@@H](Cc1ccc(NS(=O)(=O)O)cc1)c1ncc(-c2ccccc2)s1] | 0.308 |
| | (S)-4-(2-(tert-Butoxycarbonylamino)-2-(5-phenylthiazol-2-yl) | |
| | ethyl)-phenyl sulfamic acid | |
| AA73 | 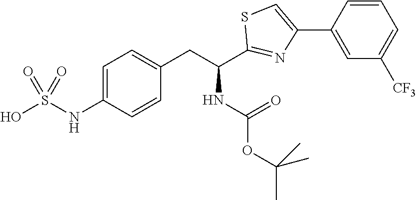 [SMILES: CC(C)(C)OC(=O)N[C@@H](Cc1ccc(NS(=O)(=O)O)cc1)c1nc(-c2cccc(C(F)(F)F)c2)cs1] | 0.253 |
| | 4-((S)-2-(tert-Butoxycarbonylamino)-2-(4-(3-(trifluoromethyl)phenyl) | |
| | thiazol-2-yl)ethyl)-phenylsulfamic acid | |
| AA74 | 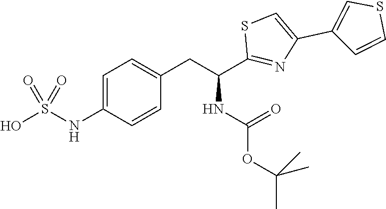 [SMILES: CC(C)(C)OC(=O)N[C@@H](Cc1ccc(NS(=O)(=O)O)cc1)c1nc(-c2ccsc2)cs1] | 0.045 |
| | 4-((S)-2-(tert-Butoxycarbonylamino)-2-(4-(thiophen-3- | |
| | yl)thiazol-2-yl)ethyl)phenyl sulfamic acid | |
| AA75 | 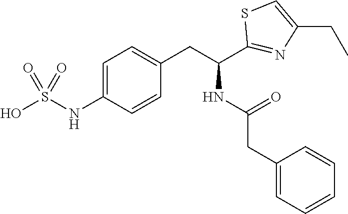 [SMILES: CCc1csc([C@H](Cc2ccc(NS(=O)(=O)O)cc2)NC(=O)Cc2ccccc2)n1] | 0.05 |
| | (S)-{4-[2-(4-Ethylthiazol-2-yl)-2- | |
| | (phenylacetylamido)ethyl]-phenyl}sulfamic acid | |
| AA76 | 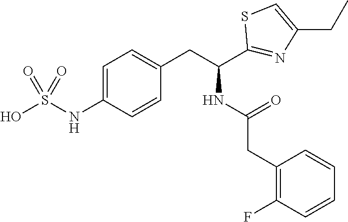 [SMILES: CCc1csc([C@H](Cc2ccc(NS(=O)(=O)O)cc2)NC(=O)Cc2ccccc2F)n1] | 0.012 |
| | (S)-4-(2-(4-Ethylthiazol-2-yl)-2-(2-(2- | |
| | fluorophenyl)acetamido)ethyl)phenyl-sulfamic acid | |
| AA77 | 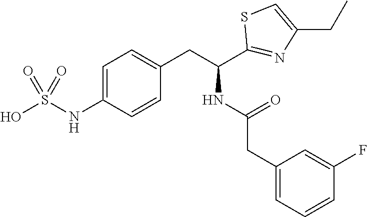 [SMILES: CCc1csc([C@H](Cc2ccc(NS(=O)(=O)O)cc2)NC(=O)Cc2cccc(F)c2)n1] | 0.0003 |
| | (S)-4-(2-(4-Ethylthiazol-2-yl)-2-(2-(3-fluorophenyl) | |
| | acetamido)ethyl)phenyl-sulfamic acid | |
| AA78 | 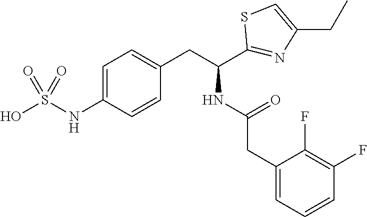 [SMILES: CCc1csc([C@H](Cc2ccc(NS(=O)(=O)O)cc2)NC(=O)Cc2cccc(F)c2F)n1] | 0.028 |
| | (S)-4-(2-(2-(2,3-Difluorophenyl)acetamido)-2-(4- | |
| | ethylthiazol-2-yl)ethyl)phenyl-sulfamic acid | |
| AA79 | 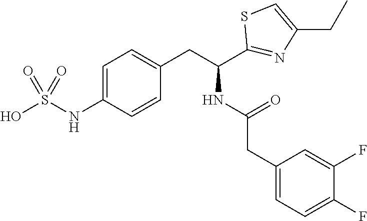 [SMILES: CCc1csc([C@H](Cc2ccc(NS(=O)(=O)O)cc2)NC(=O)Cc2ccc(F)c(F)c2)n1] | 0.075 |
| | (S)-4-(2-(2-(3,4-Difluorophenyl)acetamido)-2-(4- | |
| | ethylthiazol-2-yl)ethyl)phenyl-sulfamic acid | |
| AA80 | 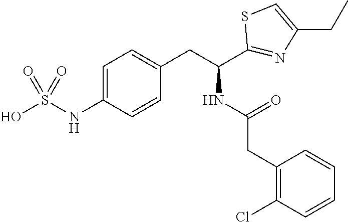 [SMILES: CCc1csc([C@H](Cc2ccc(NS(=O)(=O)O)cc2)NC(=O)Cc2ccccc2Cl)n1] | 0.056 |
| | (S)-4-(2-(2-(2-Chlorophenyl)acetamido)-2-(4- | |
| | ethylthiazol-2-yl)ethyl)phenyl-sulfamic acid | |
| AA81 | 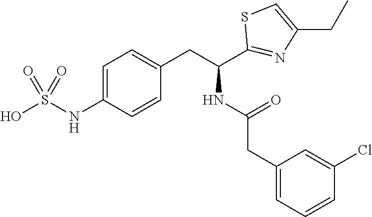 [SMILES: CCc1csc([C@H](Cc2ccc(NS(=O)(=O)O)cc2)NC(=O)Cc2cccc(Cl)c2)n1] | 0.033 |
| | (S)-4-(2-(2-(3-Chlorophenyl)acetamido)-2-(4- | |
| | ethylthiazol-2-yl)ethyl)phenyl-sulfamic acid | |
| AA82 | 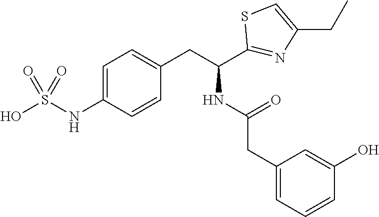 [SMILES: CCc1csc([C@H](Cc2ccc(NS(=O)(=O)O)cc2)NC(=O)Cc2cccc(O)c2)n1] | 0.04 |
| | (S)-4-(2-(4-Ethylthiazol-2-yl)-2-(2-(3-hydroxyphenyl) | |
| | acetamido)ethyl)phenyl-sulfamic acid | |
| AA83 | 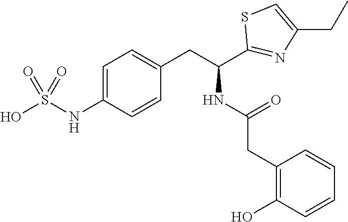 [SMILES: CCc1csc([C@H](Cc2ccc(NS(=O)(=O)O)cc2)NC(=O)Cc2ccccc2O)n1] | 0.014 |
| | (S)-4-(2-(4-Ethylthiazol-2-yl)-2-(2-(2-methoxyphenyl) | |
| | acetamido)ethyl)phenyl-sulfamic acid | |
| AA84 | 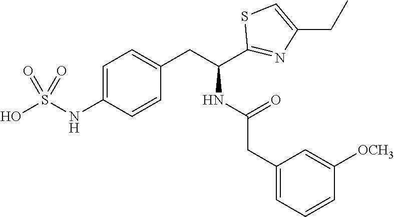 [SMILES: CCc1csc([C@H](Cc2ccc(NS(=O)(=O)O)cc2)NC(=O)Cc2cccc(OC)c2)n1] | 0.008 |
| | (S)-4-(2-(4-Ethylthiazol-2-yl)-2-(2-(3-methoxyphenyl) | |
| | acetamido)ethyl)phenyl-sulfamic acid | |
| AA85 | 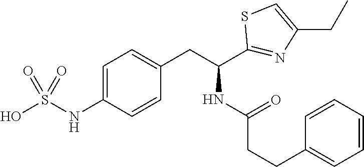 [SMILES: CCc1csc([C@H](Cc2ccc(NS(=O)(=O)O)cc2)NC(=O)CCc2ccccc2)n1] | 0.002 |
| | (S)-4-(2-(4-Ethylthiazol-2-yl)-2-(3-phenylpropanamido) | |
| | ethyl)phenylsulfamic acid | |
| AA86 | 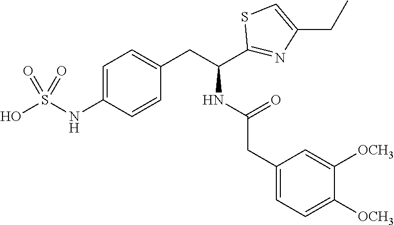 [SMILES: CCc1csc([C@H](Cc2ccc(NS(=O)(=O)O)cc2)NC(=O)Cc2ccc(OC)c(OC)c2)n1] | 0.028 |
| | (S)-4-(2-(2-(3,4-Dimethoxyphenyl)acetamido)-2- | |
| | (4-ethylthiazol-2-yl)ethyl)-phenylsulfamic acid | |
| AA87 | 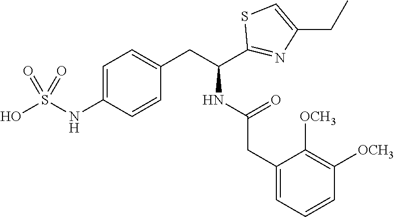 [SMILES: CCc1csc([C@H](Cc2ccc(NS(=O)(=O)O)cc2)NC(=O)Cc2cccc(OC)c2OC)n1] | 0.037 |
| | (S)-4-(2-(2-(2,3-Dimethoxyphenyl)acetamido)-2- | |
| | (4-ethylthiazol-2-yl)ethyl)-phenylsulfamic acid | |
| AA88 | 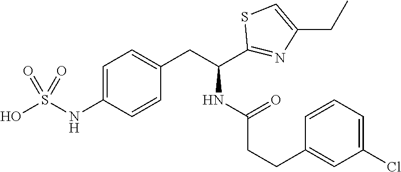 [SMILES: CCc1csc([C@H](Cc2ccc(NS(=O)(=O)O)cc2)NC(=O)CCc2cccc(Cl)c2)n1] | 0.0002 |
| | (S)-4-(2-(3-(3-Chlorophenyl)propanamido)-2-(4- | |
| | ethylthiazol-2-yl)ethyl)phenyl-sulfamic acid | |
| AA89 | 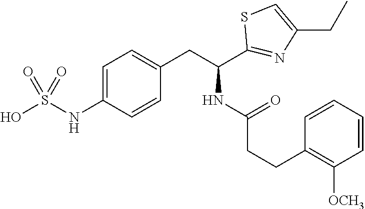 [SMILES: CCc1csc([C@H](Cc2ccc(NS(=O)(=O)O)cc2)NC(=O)CCc2ccccc2OC)n1] | 0.003 |
| | (S)-4-(2-(4-Ethylthiazol-2-yl)-2-(3-(2-methoxyphenyl) | |
| | propanamido)ethyl)phenyl-sulfamic acid | |
| AA90 | 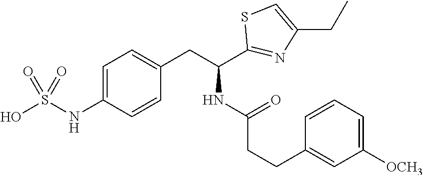 [SMILES: CCc1csc([C@H](Cc2ccc(NS(=O)(=O)O)cc2)NC(=O)CCc2cccc(OC)c2)n1] | 0.01 |
| | (S)-4-(2-(4-Ethylthiazol-2-yl)-2-(3-(3-methoxyphenyl) | |
| | propanamido)ethyl)phenyl-sulfamic acid | |
| AA91 | 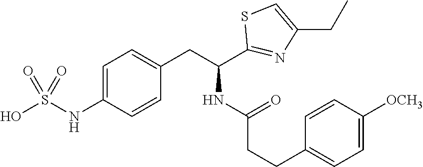 [SMILES: CCc1csc([C@H](Cc2ccc(NS(=O)(=O)O)cc2)NC(=O)CCc2ccc(OC)cc2)n1] | 0.006 |
| | (S)-4-(2-(4-Ethylthiazol-2-yl)-2-(3-(4-methoxyphenyl) | |
| | propanamido)ethyl)phenyl-sulfamic acid | |
| AA92 | 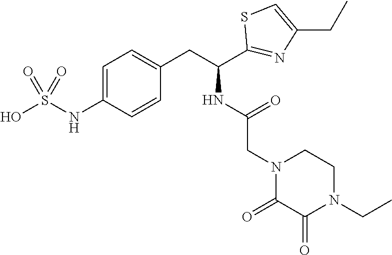 [SMILES: CCc1csc([C@H](Cc2ccc(NS(=O)(=O)O)cc2)NC(=O)CN2CCN(CC)C(=O)C2=O)n1] | 0.002 |
| | (S)-4-{2-[2-(4-Ethyl-2,3-dioxopiperazin-1-yl)acetamide]-2- | |
| | (4-ethylthiazol-2-yl)ethyl}phenylsulfamic acid | |
| AA93 | 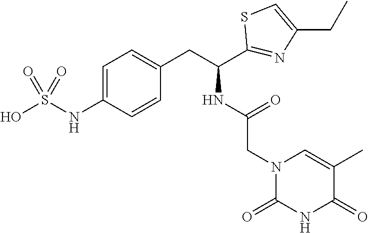 [SMILES: CCc1csc([C@H](Cc2ccc(NS(=O)(=O)O)cc2)NC(=O)Cn2cc(C)c(=O)[nH]c2=O)n1] | 0.002 |
| | (S)-4-{2-(4-Ethylthiazol-2-yl)-2-[2-(5-methyl-2,4-dioxo-3,4- | |
| | dihydropyrimidin-1(2H)-yl)acetamide]ethyl} | |
| | phenylsulfamic acid | |
| AA94 | 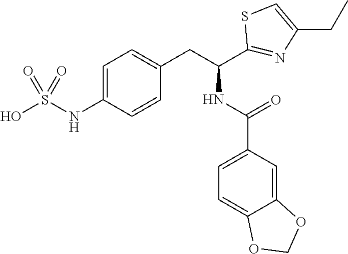 [SMILES: CCc1csc([C@H](Cc2ccc(NS(=O)(=O)O)cc2)NC(=O)c2ccc3c(c2)OCO3)n1] | 0.042 |
| | (S)-4-[2-(Benzo[d][1,3]dioxole-5-carboxamido)-2-(4- | |
| | ethylthiazol-2-yl)ethyl]phenylsulfamic acid | |
| AA95 | 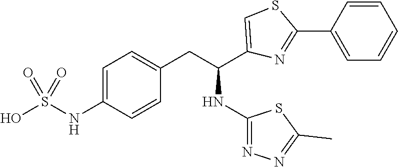 [SMILES: Cc1nnc(N[C@@H](Cc2ccc(NS(=O)(=O)O)cc2)c2csc(-c3ccccc3)n2)s1] | 0.003 |
| | (S)-4-(2-(5-methyl-1,3,4-thiadiazol-2-ylamino)-2-(2- | |
| | phenylthiazol-4-yl)ethyl)phenylsulfamic acid | |
| AA96 | 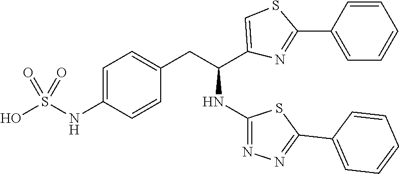 [SMILES: O=S(=O)(O)Nc1ccc(C[C@H](Nc2nnc(-c3ccccc3)s2)c2csc(-c3ccccc3)n2)cc1] | 0.046 |
| | (S)-4-(2-(5-Phenyl-1,3,4-thiadiazol-2-ylamino)-2-(2- | |
| | phenylthiazol-4-yl)ethyl)-phenylsulfamic acid | |
| AA97 | 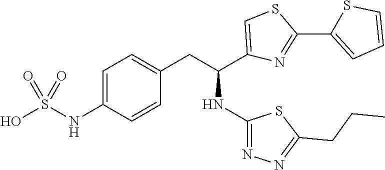 [SMILES: CCCc1nnc(N[C@@H](Cc2ccc(NS(=O)(=O)O)cc2)c2csc(-c3cccs3)n2)s1] | 0.0002 |
| | 4-((S)-2-(5-Propyl-1,3,4-thiadiazol-2-ylamino)-2-(2- | |
| | (thiophen-2-yl)thiazol-4-yl)ethyl)phenylsulfamic acid | |
| AA98 | 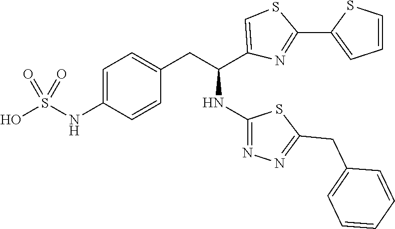 [SMILES: O=S(=O)(O)Nc1ccc(C[C@H](Nc2nnc(Cc3ccccc3)s2)c2csc(-c3cccs3)n2)cc1] | 0.0006 |
| | 4-(S)-2-(5-Benzyl-1,3,4-thiadiazol-2-ylamino)-2-(2- | |
| | (thiophen-2-yl)thiazol-4-yl)ethyl)phenylsulfamic acid | |
| AA99 | 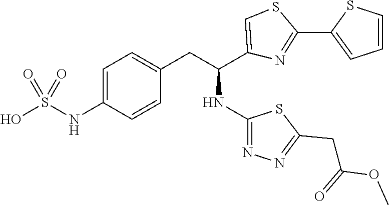 [SMILES: COC(=O)Cc1nnc(N[C@@H](Cc2ccc(NS(=O)(=O)O)cc2)c2csc(-c3cccs3)n2)s1] | 0.002 |
| | 4-((S)-2-(5-[(Methoxycarbonyl)methyl)-1,3,4-thiadiazol- | |
| | 2-ylamino)-2-(2-(thiophen-2-yl)thiazol-4- | |
| | yl)ethyl)phenylsulfamic acid | |
| AA100 | 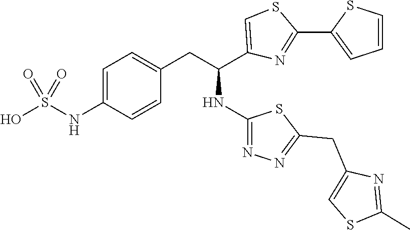 [SMILES: Cc1nc(Cc2nnc(N[C@@H](Cc3ccc(NS(=O)(=O)O)cc3)c3csc(-c4cccs4)n3)s2)cs1] | 9 × 10⁻⁶ |
| | 4-((S)-2-(5-((2-Methylthiazol-4-yl)methyl)-1,3,4-thiadiazol-2-ylamino)-2- | |
| | (2-(thiophen-2-yl)thiazol-4-yl)ethyl)phenylsulfamic acid | |

### Example 2. A Tie-2 Activator in Combination with Prostaglandin Reduced Intraocular Pressure in Human Patients with Primary Open Angle Glaucoma

A randomized, double-masked Phase 1b trial was designed to assess safety, tolerability, and efficacy of a Tie-2 activator (Compound 1) as an adjunctive therapy to prostaglandins for treatment of ocular hypertension (OHT) or primary open angle glaucoma (POAG). The study assessed the effects of increasing concentrations of Compound 1 dosed topically as eye drops (5 mg/mL QD, 15 mg/mL QD, 40 mg/mL QD, and 40 mg/mL BID) in 4 sequential cohorts of 12 subjects, randomized 3:1 to receive Compound 1 or placebo, for 7 days. Conjunctival hyperemia and IOP were assessed at 0 (pre-dose), 2 hours, 4 hours, and 8 hours post-dose on Day −1 (baseline), Day 1 (first day of Compound 1 dosing), and Day 7 (last day of dosing).

Inclusion criteria for this study were as follows:
Males and non-pregnant, non-lactating females
Aged 18 to 80 years (inclusive).
Body mass index of ≤35 kg/m² or, if outside the range, considered not clinically significant by the Investigator.
IOP≥17 mmHg in at least one eye (must be the same eye) on two separate days during Screening and IOP≤27 mmHg in both eyes on two separate days during Screening.
≤36 mmHg for Cohort 5 at all timepoints on Baseline (Day −1).
Central corneal thickness of 480 to 600 μm (inclusive).
Must be willing and able to communicate and participate in the whole study.
Must provide written informed consent.
Diagnosis of OAG or OHT.
Must be on current prostaglandin therapy for at least 4 weeks prior to screening. Subjects receiving additional carbonic-anhydrase inhibition therapy or cholinergic agonist therapy were washed-out for at least 7 days prior to Day −1; subjects currently on other IOP reducing therapy are not eligible.

Exclusion criteria for this study were as follows:
Smokers of more than 10 cigarettes per day or equivalent.
Women who are pregnant or breast-feeding or intending to become pregnant within the next 3 months.
Women of child-bearing potential who are not on an acceptable method of birth control (e.g., 1 highly effective method [less than 1% failure rate] of contraception, or a combination of 2 effective methods of contraception).
Use of systemic or ocular corticosteroid treatment within 90 days of screening or likely to require use thereof during the study period. Dermatologic corticosteroids (other than facial dermatologic corticosteroids) and intranasal steroids were allowed. Stable dose of inhaled steroids was allowed.
History of any clinically significant disease or disorder which, in the opinion of the Investigator, may put the subject at risk because of participation in the study, influence the subject's ability to participate in the study, or interfere with interpretation of the subject's study results. Subjects with stable baseline medical conditions on established medication may be included as per the judgement of the Investigator; e.g., well controlled hypertension, type 2 diabetes, or mild, stable asthma not requiring treatment with inhaled corticosteroids. Use of medical marijuana is exclusionary.
Use of an investigational medication or device within 30 days or 5-half-lives of the investigational medication, whichever is longer, preceding Day 1, or ongoing or scheduled participation in another investigational study during the present study through the last visit.
Subjects having a pseudoexfoliation or pigment dispersion component glaucoma, or have a history of angle closure or narrow iridocorneal angles (including previous peripheral iridotomy), or have evidence of angle narrowing or angle closure on gonioscopic examination, or have had previous glaucoma incisional or laser surgery, or refractive surgery less than one year prior to screening, or have a scotoma within 10 degrees of the fovea on a visual field performed within 6 months of screening. History of refractive surgery>1 year prior to screening is acceptable per investigator judgement.
Use of any ocular drops (including lubricating drops/artificial tears) during screening period or need for ocular drops during duration of study participation (other than PM prostaglandin eye drops).
Clinically significant eye trauma within 6 months of screening.
Any intraocular ophthalmic procedure within 6 months of screening.
Any ocular inflammation within 90 days of screening or a history of recurrent uveitis in either eye.
Subjects with any known chronic ocular disease (other than incipient cataract or refractive error, or OAG/OHT).
Any condition preventing valid applanation tonometry measurement; e.g., clinically significant corneal disease, refractive surgery.
Unable to discontinue contact lens wear during the study and screening assessments.
Visual acuity (VA) worse than 20/100 in either eye.
Subjects who are study site employees, or immediate family members of a study site or sponsor employee.
History of any drug or alcohol abuse in the past 2 years.
Regular alcohol consumption in males>21 units per week and females>14 units per week (1 unit=½ pint beer, 25 mL of 40% spirit, or a 125 mL glass of wine).
Presence or history of a clinically significant allergy, including drug hypersensitivity requiring treatment, as judged by the Investigator.
Failure to satisfy the Investigator of fitness to participate for any other reason.

In this study, 36 human subjects with OHT/POAG and baseline IOP measurements between 17 and 27 mmHg (inclusive) undergoing once-daily prostaglandin therapy were recruited. Subjects were randomized 3:1 to once-daily, 40 mg/mL Compound 1 (27 subjects), or placebo (9 subjects) in the morning (AM) for 7 days while continuing once-daily prostaglandin therapy in the evening (PM). Compound 1 (40 mg/mL) was a sodium salt formulated in 15% HPβCD and 1% mannitol. The Compound 1 ophthalmic solution was adjusted to a pH of 6.5-7.5 with 1N hydrochloric acid and/or IN sodium hydroxide prior to sterile filtration. Placebo was the same solution as the vehicle for the Compound 1 ophthalmic solution. Each dose of study medication (Compound 1 or placebo) was administered as one drop (30 μL) of dosing solution per eye in each subject. Compound 1 was administered to each eye (except on Day 1 and 2 in Cohort 1 in which only the right eye received Compound 1) by the topical ocular route using a dropper bottle to deliver eye drops. Compound 1 was administered for 7 days as a single morning dose daily. Prostaglandin therapy, e.g., latanoprost 0.005% (50 μg/mL), travoprost 0.004% (40 μg/mL), bimatoprost 0.01% (100 μg/mL) or 0.03% (300 μg/mL), tafluprost 0.0015% (15 μg/mL), or latanoprostene bunod 0.024% (240 μg/mL) was administered once daily in the evening in both eyes.

IOP was measured pre-dose (0 hr), 2 hrs, 4 hrs, and 8 hrs post-dose on Day −1, Day 1, and Day 7. Diurnal time point assessments of IOP was performed on Day 1 and Day 7 in both eyes (pre-dose and 2, 4, and 8 hours post-dose). Visual field testing, conjunctival hyperemia and IOP were also assessed. IOP was assessed by Goldmann applanation tonometry in both eyes at screening, baseline, and during treatment as indicated. IOP are conducted as follows: measure twice, with 3rd measure if discordant.

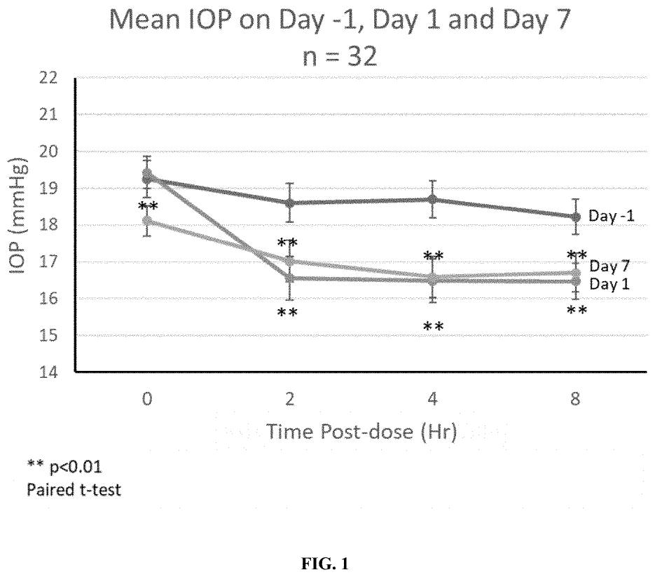

FIG. 1 illustrates changes in mean IOP on Day −1, Day 1, and Day 7 for subjects treated with Compound 1 in combination with prostaglandin therapy. A statistically significant decrease in IOP from baseline was observed at all post-dose timepoints, including the Day 7 pre-dose. The diurnal mean reduction on Day 7 was −1.58 mmHg (p<0.0001) compared to 0.06 mmHg for placebo (p=0.462).

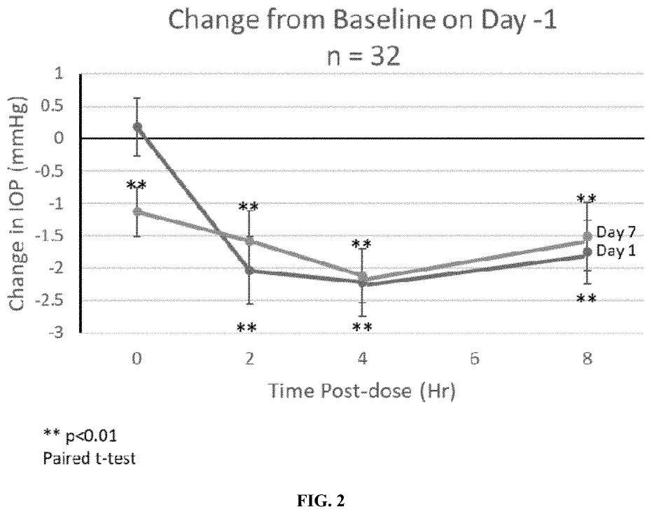

FIG. 2 illustrates changes in mean IOP from baseline on Day 1 and Day 7.

Compound 1 plus prostaglandin versus prostaglandin alone (with placebo) produced a statistically significant decrease in IOP (placebo-corrected change from baseline) on Day 7 at 0, 4, and 8 hours post-dose (0 hrs −2.26 mmHg, p=0.007; 2 hrs −1.24 mmHg, p=0.138; 4 hrs −1.47 mmHg; p=0.048; 8 hrs −1.80 mmHg; p=0.041), which suggested a consistent and persistent benefit of including Compound 1 with prostaglandin therapy.

TABLE 2 shows a categorical analysis of diurnal IOP reduction at Day 7. About 40% of patients had diurnal IOP<16 mmHg and ≥2 mmHg reduction in diurnal IOP.

TABLE 2

| Day 7, Diurnal Mean using | Compound 1 (40 mg/mL QD) + | |
|---|---|---|
| Pre-dose and 2, 4, and 8 hrs | Prostaglandin (N = 32) | |
| IOP < 16 mmHg | 12 | (37.5%) |
| IOP Decrease ≥ 2 mmHg | 12 | (37.5%) |
| IOP Decrease ≥ 3 mmHg | 5 | (15.6%) |
| IOP Decrease ≥ 4 mmHg | 3 | (9.4%) |

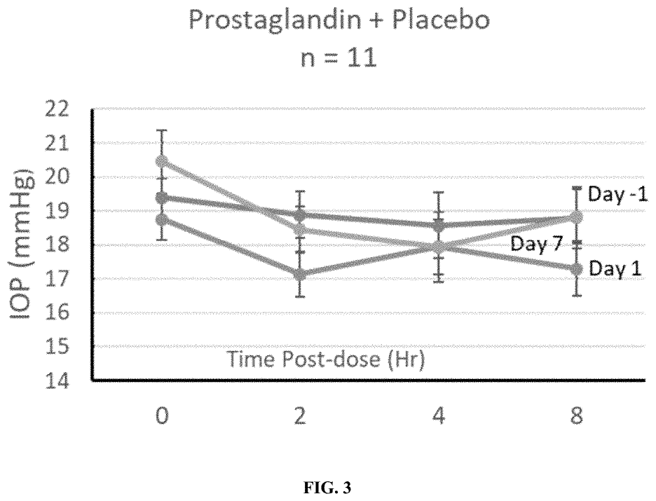

TABLE 3 shows placebo corrected mean IOP change from baseline (Day −1) at 0, 2, 4, and 8 hours at Day 1 and Day 7. FIG. 3 illustrates changes in mean IOP on Day −1, Day 1, and Day 7 for subjects treated with prostaglandin and placebo. At Day 7, placebo corrected change from baseline ranged from −1.24 to −2.26 and was statistically significant at 3 of 4 time points. Placebo corrected diurnal mean IOP reduction at Day 7 was −1.66 (p=0.0103).

TABLE 3

| | 0 Hr | 2 Hr | 4 Hr | 8 Hr |
|---|---|---|---|---|
| Day 1 | | | | |
| Difference from Placebo | 0.76 | −0.40 | −1.53 | −0.47 |
| 95% 2-sided CI | (−0.68, 2.21) | (−2.24, 1.45) | (−3.51, 0.44) | (−2.29, 1.34) |
| p-value | 0.2924 | 0.6670 | 0.1248 | 0.6008 |
| Day 7 | | | | |
| Difference from Placebo | −2.26 | −1.24 | −1.47 | −1.80 |
| 95% 2-sided CI | (−3.86, −0.65) | (−2.89, 0.42) | (−2.92, −0.01) | (−3.52. −0.08) |
| p-value | 0.0072 | 0.1384 | 0.0484 | 0.0407 |

TABLE 4 shows categories of average of study eye and fellow eye intraocular pressure (IOP, mmHg) by visit and time point study.

TABLE 4

| | |
|---|---|
| Compound 1 | |
| (40 mg/mL QD) + | Placebo + |
| Prostaglandin | Prostaglandin |
| (N = 32) | (N = 11) |

Day 7, Pre-dose

| IOP < 16 mmHg | Not Reported | | Not Reported | |
|---|---|---|---|---|
| IOP Decrease ≥ 2 mmHg | 11 | (34.4%) | 1 | (9.1%) |
| IOP Decrease ≥ 3 mmHg | 5 | (15.6%) | 1 | (9.1%) |
| IOP Decrease ≥ 4 mmHg | 2 | (6.3%) | 1 | (9.1%) |

Day 7, 2 Hour

| | | | | |
|---|---|---|---|---|
| IOP < 16 mmHg | 8 | (25.0%) | 1 | (9.1%) |
| IOP Decrease ≥ 2 mmHg | 9 | (28.1%) | 2 | (18.2%) |
| IOP Decrease ≥ 3 mmHg | 7 | (21.9%) | 1 | (9.1%) |

| | | |
|---|---|---|
| IOP Decrease ≥ 4 mmHg | Not Reported | Not Reported |

Day 7, 4 Hour

| | | | | |
|---|---|---|---|---|
| IOP < 16 mmHg | 14 | (43.8%) | 1 | (9.1%) |
| IOP Decrease ≥ 2 mmHg | 17 | (53.1%) | 2 | (18.2%) |
| IOP Decrease ≥ 3 mmHg | 12 | (37.5%) | 1 | (9.1%) |

| | | |
|---|---|---|
| IOP Decrease ≥ 4 mmHg | Not Reported | Not Reported |

Day 7, 8 Hour

| | | | | |
|---|---|---|---|---|
| IOP < 16 mmHg | 15 | (46.9%) | 1 | (9.1%) |
| IOP Decrease ≥ 2 mmHg | 14 | (43.8%) | 1 | (9.1%) |
| IOP Decrease ≥ 3 mmHg | 11 | (34.4%) | 1 | (9.1%) |

| | | |
|---|---|---|
| IOP Decrease ≥ 4 mmHg | Not Reported | Not Reported |

Not reported indicates that number of subjects = 0 for at least one of the treatment groups. Diurnal mean summaries are not provided for Placebo + Prostaglandin group.
Note:
N represents the total number of subjects within the population
Percentages are based on N (no subjects have missing data at any visit).
Decreases calculated from baseline. Baseline refers to the Day −1 data at the corresponding time point (i.e., diurnally adjusted baseline).
Change from baseline is defined as Visit Value − Baseline Value.

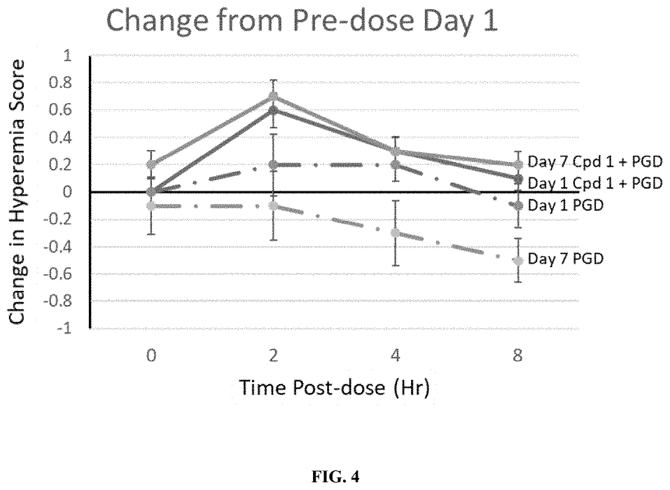

FIG. 4 shows the change in hyperemia score from pre-dose Day 1 to Day 7 for Compound 1 with prostaglandin (Cpd 1+PG) or prostaglandin with placebo (PG).

Conjunctival hyperemia scoring was as follows:

None (0)—Normal. Appears white with a small number of conjunctival blood vessels easily observed.
Minimal (1)—Trace pinkish color of either the bulbar or palpebral conjunctiva.
Mild (2)—Prominent, pinkish-red color of both the bulbar and palpebral conjunctiva.
Moderate (3)—Scarlet red color of the bulbar and palpebral conjunctiva.
Severe (4)—“Beefy Red” with petechiae. Dark red bulbar and palpebral conjunctiva with or without evidence of subconjunctival hemorrhage.

Topical ocular administration of Compound 1 was well-tolerated over the 7 day study. In the dosing arm treated with Compound 1 plus prostaglandin, 18.8% of subjects experienced hyperemia compared with 9.1% of subjects in the prostaglandin-alone arm. In all cases, this hyperemia was minimal-to-mild in severity, transient in duration, and generally considered non-adverse. There were no reports of conjunctival hemorrhage or pain on instillation during the 7 days of dosing. Similar to healthy subjects, subjects treated with Compound 1 experienced a small increase in hyperemia over baseline at 2 hours, which returned to baseline levels by 8 hours post-dose.

### Example 3. A Tie-2 Activator Augments Prostaglandin-Induced IOP Decreases in Normotensive Dogs

A randomized, double-masked Phase 1b trial was designed to assess safety, tolerability, and efficacy of a Tie-2 activator (Compound 1) alone and as an adjunctive therapy to prostaglandin therapy. A single or repeated topical ocular administration of 4% Compound 1 was administered alone or with 0.005% Latanoprost to normotensive female beagle dogs. The study design is summarized in TABLE 5.

TABLE 5

| | | | | Target |
|---|---|---|---|---|
| | | | | Dose |
| | No. of | | | Volume |
| Phase | Animals | Dose Route | Formulation Dosed (OU) | (μL/eye) |
| 1 | 12 | Topical^{a} | Vehicle Control (VC) | 35 |
| 2 | 12 | Topical^{a} | 0.005% Latanoprost in MF | 35 |
| 3 | 12 | Topical^{b} | 4% Compound 1 in VC | 35 |
| 4a | 6 | Topical^{c} | 0.005% Latanoprost in MF | 35 |
| | | | and 4% Compound 1 in VC | |
| 4b | 6 | Topical^{c} | 0.005% Latanoprost in MF | 35 |
| | | | and VC | |

MF, Marketed formulation of Xalatan ®
OU, Both eyes.
VC, Vehicle control [15% hydroxypropyl-β-cyclodextrin (HPβCD) + 1% mannitol].
^{a}A 35 μL topical ocular dose administered OU on Day 1 only.
^{b}Once daily, 35 μL topical ocular dose administered OU for 5 days.
^{c}Once daily, 35 μL topical ocular dose of 0.005% Latanoprost in MF. After 5 minutes, 35 μL topical ocular dose of either 4% Compound 1 in VC (Phase 4a) or VC (Phase 4b). This dosing regimen was administered OU for 5 days.

Intraocular pressure was measured for all animals in Phase 1 on Day −1 and Day 1 at −1, 0, 1, 2, 4, 6, and 8 hours post-dose (relative to the time of dosing on Day 1), and for all animals in Phase 2 on Day 1 at −1, 0, 1, 2, 4, 6, and 8 hours post-dose. The baseline (Day −1) IOP measurements for Phase 1 were used as baseline for Phase 2. For all animals in Phases 3 and 4, IOP was measured on Day −1 and Day 1 at −1, 0, 1, 2, 4, 6, and 8 hours post-dose, at 0 and 2 hours post-dose on Day 4, and at −1, 0, 1, 2, 4, 6, and 8 hours post-dose on Day 5.

A Board-Certified Veterinary Ophthalmologist conducted ophthalmic examinations pre-dose (prior to Phase 1) on all animals and on Days −2 and 4 for Phase 3 and Days −2, 4, and 13 for Phase 4.

All dogs were preconditioned (trained) to IOP measurements procedures using a rebound tonometer (TonoVet®) for at least 4 weeks prior to the first dosing, and measurements were also performed during the wash-out periods to ensure dogs remained conditioned. This study employed a non-randomized, cross-over design, with the same group of 12 dogs used for Phases 1-3, the first 6 dogs for Phase 4a, and the remaining 6 dogs for Phase 4b. A 2-day washout period occurred between Phases 1 and 2, a 13-day washout period between Phases 2 and 3, and a 9-day washout between Phases 3 and 4.

IOP measurements were assessed for each dog between phases and had returned to at least 80% of the pre-dose baseline measurement before the start of the next phase. In all phases, dogs were administered a once-daily, 35 μL bilateral topical ocular dose of the described test article(s). In Phase 1, all dogs were administered the vehicle control (15% hydroxypropyl-β-cyclodextrin [HPβCD]+1% mannitol) on Day 1 only. In Phase 2, all dogs were administered the marketed formulation of Xalatan® (0.005% Latanoprost) on Day 1 only. In Phase 3, all dogs were administered 4% Compound 1 in the vehicle formulation for Days 1 through 5. In Phase 4, all dogs were administered a topical ocular dose of 0.005% Latanoprost (Xalatan®), followed by either 4% Compound 1 in the vehicle control formulation (Animals D0001 through D0006; Phase 4a) or vehicle control (Animals D0007 through D0012; Phase 4b) for Days 1 through 5.

A repeated topical ocular administration of 4% Compound 1 alone (Phase 3) resulted in conjunctival hyperemia in 21 of 24 eyes (score of 1+ in 13 eyes and 2+ in 8 eyes observed on Day 4) but with no discernable changes in pupil size and a normal response was observed to tropicamide. Repeated topical ocular administration of 0.005% Latanoprost, immediately followed with either topical ocular administration of vehicle control or 4% Compound 1 (Phase 4), resulted in intense miosis and failure of the pupil to dilate following topical ocular application of tropicamide. Miosis is associated with topical ocular administration of Latanoprost. Conjunctival hyperemia scores on Day 4 in eyes administered 0.005% Latanoprost and vehicle control (1+ in 10 eyes and 2+ in 2 eyes) were lesser than those observed in eyes administered 0.005% Latanoprost and 4% Compound 1 (1+ in 4 eyes, 2+ in 6 eyes, and 3+ in 2 eyes). Conjunctival hyperemia scores in eyes administered 0.005% Latanoprost and 4% Compound 1 were also greater than those observed when either drug was administered alone.

Following a single topical ocular administration, the largest decrease in IOP, as assessed by Eₘₐₓ and AUEC of baseline-corrected or percent change from baseline IOP values, was observed with administration of 0.005% Latanoprost and 4% Compound 1 (Phase 4a); followed by administration of 0.005% Latanoprost either alone (Phase 2) or with vehicle control (Phase 4b); then by administration of 4% Compound 1 alone (Phase 3); and lastly administration of vehicle control (Phase 1). Following 5 days of dosing, the IOP-lowering effects of Compound 1 alone (Phase 3) and of 0.005% Latanoprost immediately followed by vehicle or Compound 1 (Phase 4) remained consistent with the respective IOP-lowering effect on Day 1. In addition, the corrected IOP measured at time 0 on Day 5 was lower than time 0 on Day 1 for Phases 4a, suggesting that IOP lowering persists for at least 24 hours post-dose of Compound 1 when co-administered with Latanoprost.

Following topical ocular administration of the vehicle control (Phase 1), a slight decrease in IOP was observed as assessed by corrected IOP values and percentage of change from baseline; and with generally, slightly lower corrected IOP values at times 0 and 1 hour post-dose relative to values for the other treatment phases. This observation suggests that baseline corrected IOP parameter values for vehicle control may have resulted in an underestimation of effectiveness for the other treatment phases relative to vehicle control.

Overall, following topical ocular administration of the vehicle control, a decrease in IOP was observed, with Eₘₐₓ values of 10.8%/−1.85 mmHg and AUEC₀₋ₜ of 33.2 h·%/−5.36 h·mmHg. Following a single, topical, ocular administration of 4% Compound 1 in vehicle control, a greater decrease in IOP was observed compared with that following administration of vehicle control alone, with Eₘₐₓ values of 17.3%/−3.58 mmHg and AUEC₀₋ₜ of 70.3 h·%/−13.7 h·mmHg. Following a single (Day 1) topical ocular administration of 0.005% Latanoprost in MF and 4% Compound 1 in vehicle control, a greater decrease in IOP (Eₘₐₓ values of 34.7%/−5.64 mmHg and AUEC₀₋ₜ of 151 h·%/−24.6 h·mmHg) was observed compared with that following administration of 0.005% Latanoprost in MF and vehicle control (Eₘₐₓ values of 28.7%/−4.50 mmHg and AUEC₀₋ₜ of 130 h·%/−19.6% h·mmHg.

### Vehicle Control (Phases 1, 3, and 4)

The vehicle control (15% hydroxypropyl-β-cyclodextrin [HPβCD] and 1% mannitol) was formulated with sterile water for injection (SWFI).

### Animals and Husbandry

### Strain and Source

Female purebred beagles were acclimated to study conditions for at least 6 weeks prior to initial dosing. At initial dosing, animals weighed 7.8 to 8.8 kg and were 10 to 11 months of age.

### Housing

During acclimation and the test period, animals were housed in stainless steel cages. Animals were commingled, as applicable, in accordance with SOPs; animals were not commingled for at least 24 hours after test article administration to allow monitoring of any test article-related effects. Animals were individually housed for study-related procedures or behavioral or health reasons.

### Feed and Water

Certified Canine Diet #5007C (PMI, Inc.) was provided ad libitum. Water was provided fresh daily, ad libitum.

### Enrichment and Treats

For environmental and psychological enrichment, various cage and/or food enrichment (that did not require analysis) were offered in accordance with the applicable SOPs. Diets were supplemented with appropriate treats (that did not require analysis) in accordance with SOPs.

### Environment

Environmental controls for the animal room were set to maintain a temperature of 20° C. to 26° C., a relative humidity of 50%±20%, and a 12-hour light/12-hour dark cycle. As necessary, the 12-hour dark cycle was interrupted to accommodate study procedures.

### Animal Selection

Animals were not randomized. Animals were selected for use on study based on overall health, body weight, acclimation behavior, and IOP measurements.

### Identification

Animals were identified via individual cage cards and implantable microchip identification devices (IMID).

### Dose Preparation and Analysis

All formulations were prepared in a laminar flow-hood using an aseptic technique.

### Phases 1 and 4 (Vehicle Control)

The vehicle control (15% HPβCD and 1% mannitol in sterile water) was prepared and stored at ambient temperature protected from light. For Phase 4, the vehicle solution was aliquoted for daily dispensing.

Phase 1: HPβCD powder (750.24 mg) was added to SWFI (approximately 4 mL) and magnetically stirred until dissolved. Mannitol (50.46 mg) was added and magnetically stirred until a particulate-free solution was obtained. The formulation was a clear, colorless solution. The pH of the formulation was measured (initial pH: 4.88) and adjusted to pH 6.26 with 0.1N sodium hydroxide (NaOH). The formulation was brought to final 5 mL volume with SWFI, and the final pH was 6.30. The formulation was filter-sterilized using a 0.22-micron PVDF filter.

Phase 4: HPβCD powder (1501.7 mg) was added to SWFI (approximately 8 mL) and magnetically stirred until dissolved. Mannitol (100.2 mg) was added and magnetically stirred until a particulate-free solution was obtained. The formulation was a clear, colorless solution. The pH of the formulation was measured (initial pH: 7.54). The formulation was brought to final 10 mL volume with SWFI, and the final pH was 7.22. The formulation was filter-sterilized using a 0.22-micron PVDF filter.

### Phase 2 and Phase 4 (Xalatan®)

The 0.005% Latanoprost formulation (Xalatan®) was dosed as supplied. The formulation was removed from storage and allowed to reach ambient temperature. For Phase 4, the formulation was aliquoted for daily dispensing in a laminar flow-hood using aseptic technique. The formulation was a clear, colorless solution.

### Phase 3 and 4 [4% Compound 1 in Vehicle Control (15% HPβCD and 1% Mannitol)]

The Compound 1 dose formulation was prepared once for Phase 3 and once for Phase 4, was stored at ambient temperature and protected from light, and was aliquoted for daily dispensing.

Phase 3: HPβCD powder (2254.8 mg) was added to SWFI (approximately 12 mL) and magnetically stirred until dissolved. Compound 1 (639.6 mg) was added to the HPβCD solution, and the formulation was magnetically stirred for at least 2 hours and sonicated for 7 minutes to ensure thorough mixing. Mannitol (149.2 mg) was added, and the formulation was magnetically stirred for at least 30 minutes until a particulate-free solution was obtained. The formulation was a clear, colorless solution. The pH of the formulation was measured (initial pH: 9.72) and adjusted to pH 7.0 with 1.0 N hydrochloric acid (HCl). The formulation was brought to final 15 mL volume with SWFI, at a final pH of 6.90, and filter-sterilized using a 0.22-micron PVDF filter.

Phase 4: HPβCD powder (2,249.4 mg) was added to SWFI (approximately 12 mL) and magnetically stirred until dissolved. Compound 1 (641.3 mg) was added to the HPβCD solution, and the formulation was magnetically stirred for at least 2 hours and sonicated for 5 minutes to ensure thorough mixing. Mannitol (149.8 mg) was added and the formulation was magnetically stirred for at least 30 minutes until a particulate-free solution was obtained. The formulation was a clear, colorless solution. The pH of the formulation was measured (initial pH: 9.57) and adjusted to pH 6.68 with 1.0 N HCl and 0.1 N NaOH. The formulation was brought to final 15 mL volume with SWFI, at a final pH of 6.79, and filter-sterilized using a 0.22-micron PVDF filter.

### Dose Administration

Animals were not fasted.

Each formulation was administered as a topical ocular dose to the central or superior part of the cornea via a positive displacement pipette and allowed to spread across the surface of the eye. After the dose was administered, the eye was allowed to close naturally. Each animal was then restrained for approximately 1 minute to prevent rubbing of the eyes in accordance with SOP.

No irregularities or local irritation was observed at dosing.

### Observation of Animals

### Antemortem Observations

On the day of arrival, animals were observed for mortality and signs of pain and distress once (p.m.). Beginning the day after arrival, animals were observed for mortality and signs of pain and distress twice daily (a.m. and p.m.), and cageside observations for general health and appearance were performed once daily.

### Body Weights

Body weights were measured within 5 days of arrival and weekly throughout acclimation, as applicable. Animals were also weighed at the time of animal selection, on the day of the first dosing for each phase, and weekly throughout the remainder of the study, as applicable.

### Ophthalmic Examinations

A board-certified veterinary ophthalmologist conducted ophthalmic examinations pre-dose (prior to Phase 1) on all animals and on Days −2 and 4 for Phases 3 and 4. An additional ophthalmic examination was performed on Day 13 of Phase 4.

At each time point, both eyes were grossly examined and graded using a modified Hackett-McDonald Scoring System. A slit-lamp biomicroscope was used to examine the adnexa and anterior portion of each eye. In addition, eyes were dilated with a mydriatic agent (tropicamide) and the ocular fundus of each eye was examined using an indirect ophthalmoscope. The pupillary light reflex was assessed prior to pupillary dilation. Corneal fluorescein staining was performed at each interval.

### Intraocular Pressure Conditioning

Following at least 1 week of acclimation, all animals were preconditioned (trained) to IOP measurement procedures (using a TonoVet®) three times/week at approximately the same time (a.m.) each day for at least 4 weeks prior to the first day of dosing according to a study-specific procedure.

The IOP measurements were also performed during the wash-out period between phases to ensure animals remained conditioned to the procedure. Measurements occurred three times during the wash-out periods prior to Phases 3 and 4 and, when possible, the time of the IOP measurement correlated to the same time of day scheduled for IOP measurements performed after dosing. IOP measurements were at least 80% of the pre-dose baseline measurement for dogs prior to initiating the next phase. Conditioning IOP measurements were not performed between Phases 1 and 2 due to only one washout day between the two phases.

### Intraocular Pressure Measurement (Phases 1 and 2)

IOP was measured for all animals in Phase 1 on Day −1 and Day 1 at −1, 0, 1, 2, 4, 6, and 8 hours post-dose (relative to the time of dosing on Day 1); and for all animals in Phase 2 on Day 1 at −1, 0, 1, 2, 4, 6, and 8 hours post-dose. The baseline (Day −1) IOP measurements for Phase 1 were used as baseline for Phase 2. Readings were performed using a TonoVet® and 3 valid readings/eye were used to determine mean IOP measurements.

### Intraocular Pressure Measurement (Phases 3 and 4)

IOP was measured for all animals in Phases 3 and 4 on Day −1 at −1, 0, 1, 2, 4, 6, and 8 hours post-dose (day prior to dosing, based on the times for dosing on Day 1); on Day 1 at −1, 0 (immediately pre-dose), 1, 2, 4, 6, and 8 hours post-dose; on Day 4 at 0 (immediately prior to the Day 4 dose) and 2 hours post-dose; and on Day 5 at −1, 0 (immediately prior to the Day 5 dose), 1, 2, 4, 6, and 8 hours post-dose. For Phase 4, the IOP measurements post-dose were based on the second dose (Compound 1 or vehicle control). Three readings/eye were performed for each timepoint using a TonoVet®.

### Pharmacodynamic Analysis

From each animal at each occasion, three IOP measurements were collected for each eye and the mean of the three measured values (left or right eye) was used for pharmacodynamic analysis. Measurements collected at 1 hour pre-dose (the first measurement of the day) were excluded from pharmacodynamic analysis. Descriptive statistics in Phase 4 were reported separately for each of the two treatments in this phase.

The individual animal mean IOP measurements were corrected for baseline and time-dependent changes by subtracting the Day −1 measurement at each time point for each eye (in mmHg units) or by calculating the percentage change from the Day −1 measurement at each time point for each eye. As no data were collected on Day −1 of Phase 2, the Day −1 data from Phase 1 were used to perform this correction. During Phases 3 and 4, data from Day −1 of each respective phase were used to correct Days 1 and 5. IOP changes of greater than zero (treatment causing an increase in IOP) following correction were treated as zero for descriptive statistics and pharmacodynamic analysis. Corrected mean IOP measurements were analyzed in mmHg units and as a percent change from baseline.

Noncompartmental analysis was applied to the individual corrected IOP data.

The following parameters were estimated whenever possible:

Eₘₐₓ, maximum effect observed for corrected IOP;
Tₘₐₓ, time of maximum effect observed for corrected IOP;
AUEC₀₋ₜ, area under the effect-time curve from hour 0 to the last measurable corrected IOP, estimated by the linear trapezoidal rule;
Tₗₐₛₜ, time of last measurable corrected IOP; and
Number of Measurable IOPs, the number of corrected IOPs with measurable effect observed following correction.

Parameters for the combined left and right eyes were calculated by combining baseline-corrected IOP results for each eye at each sampling occasion. These parameters are not an average of the mean parameter values calculated for the right and left eyes separately. The data were computer generated and rounded appropriately for inclusion in the report. Neither the integrity nor the interpretation of the data was affected by these differences.

### Unexpected Results Relevant to Data Analysis

During Phase 3, the Day 1, third measurement for Animal D0007 at 4 hours post-dose was recorded as 143 mmHg. This value was determined to be an outlier and was excluded from descriptive statistics and pharmacodynamics analysis. The remaining two measurements were included; as such, this event did not impact the overall inferences from the pharmacodynamic portion of this study.

### Results and Discussion

### Acclimation

All animals appeared clinically healthy throughout acclimation and were released from acclimation and approved for use on the study.

### Body Weights and Doses Administered

No adverse effects on body weight were noted. The measured Compound 1 concentration for each of the Compound 1-containing formulations prepared for Phase 2 and Phase 4 were 39.8 and 38.1 mg/mL, respectively, relative to the targeted 40 mg/mL (4%) concentration.

### Clinical Observations

All animals appeared healthy and exhibited no overt signs of toxicity throughout the study.

### Ophthalmic Examinations

All 12 animals had a normal ophthalmic examination at the pre-dose interval.

For Day −2 of Phase 3 (4% Compound 1 in vehicle control), the examination findings consisted of mild (score of 1+) conjunctival hyperemia in both eyes of Animal D0010. On Day 4, the findings were limited to conjunctival hyperemia in 21 of 24 eyes (1+ in 13 eyes and 2+ in 8 eyes).

For Day −2 of Phase 4, all 12 animals had a normal ophthalmic examination. On Day 4 for dogs administered 0.005% Latanoprost and 4% Compound 1 (Animals D0001 through D0006), the findings consisted of pupillary miosis, that did not change following the topical application of tropicamide in all eyes, and conjunctival hyperemia (1+ in 4 eyes, 2+ in 6 eyes and 3+ in 2 eyes). Additionally, in the right eye of Animal D0004, a curvilinear area of disruption of the anterior face of the iris, which allowed the underlying blue color of the iris to show through, was observed. The iris was concentric with the pupil and unassociated with signs of intraocular inflammation or other iris changes. As such, the observation was considered a normal variation in the iris that was hidden in a normal fold of iris tissue and not appreciated until the test articles induced extreme miosis. On Day 4, for dogs administered 0.005% Latanoprost and vehicle control (Animals D0007 through D0012), the findings consisted of pupillary miosis, that did not change following the topical application of tropicamide in all eyes, and conjunctival hyperemia (1+ in 10 eyes and 2+ in 2 eyes). On Day 13 (8 days following the last dose), the ophthalmic findings were limited to mild (1+) conjunctival hyperemia in 4 of 12 eyes administered 0.005% Latanoprost with vehicle control; no ophthalmic findings were noted in animals administered 0.005% Latanoprost and 4% Compound 1. The previously noted disruption of the iris face in the right eye of Animal D0004 was observed to be partially obscured in a fold of the iris.

### Intraocular Pressure Readings and Pharmacodynamics

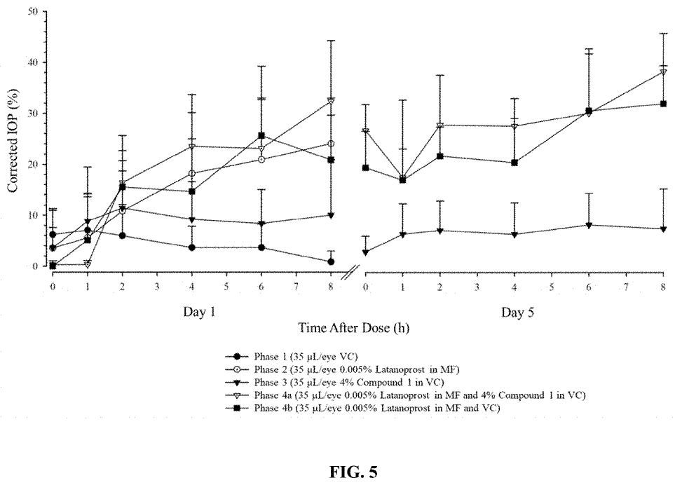

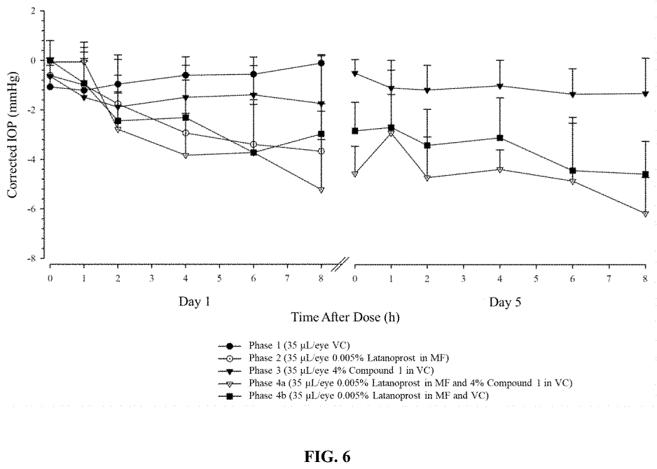

The mean corrected IOP results are presented graphically in FIG. 5 and FIG. 6. Summaries of the mean pharmacodynamics parameters are presented in TABLE 6 and TABLE 7. An overall summary of the mean pharmacodynamics parameters is presented in TABLE 8.

TABLE 6

| | | | Dose | | | | | | Number of |
|---|---|---|---|---|---|---|---|---|---|
| | | | Volume | | | Eₘₐₓ | Tₘₐₓ | AUEC₀₋ₜ | measurable |
| Phase | Day | Test Article | (μL/eye) | Eye | | (%) | (h) | (h*%) | IOPs |
| 1 | 1 | Vehicle | 35 | OD | Mean | 11.6 | 1.75 | 21.6 | 2.58 |
| | | Control | | | SD | 2.31 | 1.71 | 13.9 | 1.08 |
| | | | | | CV % | 19.9 | 97.8 | 64.3 | 41.9 |
| | | | | | N | 12 | 12 | 12 | 12 |
| | | | | OS | Mean | 14.3 | 2.64 | 49.5 | 3.83 |
| | | | | | SD | 6.78 | 2.06 | 26.8 | 1.64 |
| | | | | | CV % | 47.3 | 78.2 | 54.1 | 42.8 |
| | | | | | N | 12 | 11 | 11 | 12 |
| | | | | OU | Mean | 10.8 | 1.36 | 33.2 | 3.50 |
| | | | | | SD | 5.19 | 1.12 | 22.1 | 1.68 |
| | | | | | CV % | 48.0 | 82.1 | 66.6 | 48.0 |
| | | | | | N | 12 | 11 | 11 | 12 |
| 2 | 1 | 0.005% | 35 | OD | Mean | 25.5 | 6.83 | 121 | 4.67 |
| | | Latanoprost | | | SD | 10.6 | 1.80 | 69.5 | 1.23 |
| | | in MF | | | CV % | 41.4 | 26.4 | 57.5 | 26.4 |
| | | | | | N | 12 | 12 | 12 | 12 |
| | | | | OS | Mean | 27.8 | 6.17 | 136 | 4.58 |
| | | | | | SD | 9.30 | 1.99 | 75.1 | 1.38 |
| | | | | | CV % | 33.5 | 32.3 | 55.3 | 30.1 |
| | | | | | N | 12 | 12 | 12 | 12 |
| | | | | OU | Mean | 26.2 | 6.67 | 126 | 4.50 |
| | | | | | SD | 9.43 | 1.56 | 72.9 | 1.17 |
| | | | | | CV % | 36.0 | 23.4 | 58.0 | 25.9 |
| | | | | | N | 12 | 12 | 12 | 12 |
| 3 | 1 | 4% | 35 | OD | Mean | 16.7 | 3.73 | 67.8 | 3.75 |
| | | Compound | | | SD | 11.1 | 3.07 | 61.0 | 1.82 |
| | | 1 in Vehicle | | | CV % | 66.7 | 82.3 | 89.9 | 48.4 |
| | | Control | | | N | 12 | 11 | 11 | 12 |
| | | | | OS | Mean | 20.6 | 3.92 | 83.4 | 4.42 |
| | | | | | SD | 7.72 | 2.91 | 40.6 | 0.996 |
| | | | | | CV % | 37.5 | 74.2 | 48.7 | 22.6 |
| | | | | | N | 12 | 12 | 12 | 12 |
| | | | | OU | Mean | 17.3 | 4.92 | 70.3 | 4.33 |
| | | | | | SD | 9.38 | 3.00 | 49.7 | 1.30 |
| | | | | | CV % | 54.3 | 61.0 | 70.6 | 30.1 |
| | | | | | N | 12 | 12 | 12 | 12 |
| 3 | 5 | 4% | 35 | OD | Mean | 13.5 | 5.09 | 46.0 | 3.58 |
| | | Compound | | | SD | 5.02 | 2.55 | 25.0 | 2.11 |
| | | 1 in Vehicle | | | CV % | 37.1 | 50.0 | 54.5 | 58.8 |
| | | Control | | | N | 12 | 11 | 11 | 12 |
| | | | | OS | Mean | 17.8 | 3.42 | 60.3 | 4.00 |
| | | | | | SD | 5.11 | 2.50 | 34.5 | 1.41 |
| | | | | | CV % | 28.7 | 73.3 | 57.1 | 35.4 |
| | | | | | N | 12 | 12 | 12 | 12 |
| | | | | OU | Mean | 13.8 | 5.08 | 51.1 | 4.00 |
| | | | | | SD | 4.95 | 2.87 | 30.1 | 1.81 |
| | | | | | CV % | 35.8 | 56.6 | 58.9 | 45.2 |
| | | | | | N | 12 | 12 | 12 | 12 |
| 4a | 1 | 0.005% | 35 | OD | Mean | 34.6 | 7.67 | 151 | 4.17 |
| | | Latanoprost | | | SD | 10.9 | 0.816 | 58.1 | 0.753 |
| | | in MF and | | | CV % | 31.6 | 10.6 | 38.3 | 18.1 |
| | | 4% | | | N | 6 | 6 | 6 | 6 |
| | | Compound | | OS | Mean | 35.9 | 6.33 | 153 | 3.83 |
| | | 1 in Vehicle | | | SD | 7.31 | 1.97 | 36.8 | 0.408 |
| | | Control | | | CV % | 20.4 | 31.0 | 24.1 | 10.6 |
| | | | | | N | 6 | 6 | 6 | 6 |
| | | | | OU | Mean | 34.7 | 7.67 | 151 | 4.33 |
| | | | | | SD | 8.62 | 0.816 | 44.0 | 0.516 |
| | | | | | CV % | 24.9 | 10.6 | 29.2 | 11.9 |
| | | | | | N | 6 | 6 | 6 | 6 |
| 4a | 5 | 0.005% | 35 | OD | Mean | 38.8 | 7.33 | 231 | 5.83 |
| | | Latanoprost | | | SD | 9.05 | 1.63 | 84.3 | 0.408 |
| | | in MF and | | | CV % | 23.3 | 22.3 | 36.4 | 7.00 |
| | | 4% | | | N | 6 | 6 | 6 | 6 |
| | | Compound | | OS | Mean | 38.8 | 6.67 | 220 | 5.67 |
| | | 1 in Vehicle | | | SD | 6.25 | 2.42 | 48.2 | 0.516 |
| | | Control | | | CV % | 16.1 | 36.3 | 21.9 | 9.11 |
| | | | | | N | 6 | 6 | 6 | 6 |
| | | | | OU | Mean | 38.3 | 8.00 | 226 | 5.67 |
| | | | | | SD | 7.46 | 0.00 | 65.7 | 0.516 |
| | | | | | CV % | 19.5 | 0.00 | 29.1 | 9.11 |
| | | | | | N | 6 | 6 | 6 | 6 |
| 4b | 1 | 0.005% | 35 | OD | Mean | 27.6 | 6.33 | 120 | 4.50 |
| | | Latanoprost | | | SD | 11.7 | 1.51 | 34.9 | 0.548 |
| | | in MF and | | | CV % | 42.2 | 23.8 | 29.2 | 12.2 |
| | | Vehicle | | | N | 6 | 6 | 6 | 6 |
| | | Control | | OS | Mean | 31.0 | 5.50 | 141 | 4.33 |
| | | | | | SD | 12.7 | 2.66 | 51.0 | 0.816 |
| | | | | | CV % | 41.0 | 48.4 | 36.1 | 18.8 |
| | | | | | N | 6 | 6 | 6 | 6 |
| | | | | OU | Mean | 28.7 | 6.00 | 130 | 4.33 |
| | | | | | SD | 12.0 | 1.26 | 41.0 | 0.816 |
| | | | | | CV % | 41.8 | 21.1 | 31.6 | 18.8 |
| | | | | | N | 6 | 6 | 6 | 6 |
| 4b | 5 | 0.005% | 35 | OD | Mean | 34.1 | 5.00 | 194 | 6.00 |
| | | Latanoprost | | | SD | 3.53 | 1.67 | 25.4 | 0.00 |
| | | in MF and | | | CV % | 10.4 | 33.5 | 13.1 | 0.00 |
| | | Vehicle | | | N | 6 | 6 | 6 | 6 |
| | | Control | | OS | Mean | 37.6 | 6.33 | 193 | 5.67 |
| | | | | | SD | 10.6 | 2.34 | 59.6 | 0.516 |
| | | | | | CV % | 28.1 | 36.9 | 31.0 | 9.11 |
| | | | | | N | 6 | 6 | 6 | 6 |
| | | | | OU | Mean | 34.1 | 5.33 | 193 | 6.00 |
| | | | | | SD | 8.30 | 2.07 | 42.2 | 0.00 |
| | | | | | CV % | 24.4 | 38.7 | 21.8 | 0.00 |
| | | | | | N | 6 | 6 | 6 | 6 |

TABLE 7

| | | | Dose | | | | | | | Number of |
|---|---|---|---|---|---|---|---|---|---|---|
| | | | Volume | | | Eₘₐₓ | Tₘₐₓ | AUEC₀₋ₜ | Tₗₐₛₜ | measurable |
| Phase | Day | Test Article | (μL/eye) | Eye | | (%) | (h) | (h*%) | (h) | IOPs |
| 1 | 1 | Vehicle | 35 | OD | Mean | −1.97 | 1.75 | −3.56 | 3.83 | 2.58 |
| | | Control | | | SD | 0.437 | 1.71 | 2.31 | 1.95 | 1.08 |
| | | | | | CV % | −22.2 | 97.8 | −65.1 | 50.8 | 41.9 |
| | | | | | N | 12 | 12 | 12 | 12 | 12 |
| | | | | OS | Mean | −2.42 | 1.64 | −7.97 | 6.36 | 3.83 |
| | | | | | SD | 1.22 | 1.86 | 4.14 | 1.50 | 1.64 |
| | | | | | CV % | −50.3 | 114 | −52.0 | 23.6 | 42.8 |
| | | | | | N | 12 | 11 | 11 | 11 | 12 |
| | | | | OU | Mean | −1.85 | 1.00 | −5.36 | 5.36 | 3.50 |
| | | | | | SD | 0.895 | 1.18 | 3.57 | 2.38 | 1.68 |
| | | | | | CV % | −48.4 | 118 | −66.5 | 44.3 | 48.0 |
| | | | | | N | 12 | 11 | 11 | 11 | 12 |
| 2 | 1 | 0.005% | 35 | OD | Mean | −4.03 | 5.83 | −19.5 | 8.00 | 4.67 |
| | | Latanoprost | | | SD | 2.00 | 2.48 | 12.4 | 0.00 | 1.23 |
| | | in MF | | | CV % | −49.6 | 42.5 | −63.5 | 0.00 | 26.4 |
| | | | | | N | 12 | 12 | 12 | 12 | 12 |
| | | | | OS | Mean | −4.44 | 6.17 | −21.9 | 8.00 | 4.58 |
| | | | | | SD | 1.76 | 1.99 | 12.9 | 0.00 | 1.38 |
| | | | | | CV % | −39.6 | 32.3 | −59.2 | 0.00 | 30.1 |
| | | | | | N | 12 | 12 | 12 | 12 | 12 |
| | | | | OU | Mean | −4.17 | 6.17 | −20.2 | 8.00 | 4.50 |
| | | | | | SD | 1.81 | 1.99 | 12.8 | 0.00 | 1.17 |
| | | | | | CV % | −43.5 | 32.3 | −63.0 | 0.00 | 25.9 |
| | | | | | N | 12 | 12 | 12 | 12 | 12 |
| 3 | 1 | 4% | 35 | OD | Mean | −2.92 | 3.73 | −11.4 | 7.64 | 3.75 |
| | | Compound | | | SD | 2.00 | 3.07 | 10.7 | 0.809 | 1.82 |
| | | 1 in Vehicle | | | CV % | −68.4 | 82.3 | −93.3 | 10.6 | 48.4 |
| | | Control | | | N | 12 | 11 | 11 | 11 | 12 |
| | | | | OS | Mean | −2.28 | 5.09 | −7.85 | 7.09 | 3.58 |
| | | | | | SD | 0.919 | 2.55 | 4.55 | 1.38 | 2.11 |
| | | | | | CV % | −40.4 | 50.0 | −58.0 | 19.4 | 58.8 |
| | | | | | N | 12 | 11 | 11 | 11 | 12 |
| | | | | OU | Mean | −3.58 | 3.92 | −13.7 | 7.50 | 4.42 |
| | | | | | SD | 1.58 | 2.91 | 6.94 | 0.905 | 0.996 |
| | | | | | CV % | −44.2 | 74.2 | −50.6 | 12.1 | 22.6 |
| | | | | | N | 12 | 12 | 12 | 12 | 12 |
| 3 | 5 | 4% | 35 | OD | Mean | −3.03 | 3.92 | −10.1 | 6.67 | 4.00 |
| | | Compound | | | SD | 0.948 | 2.78 | 6.16 | 2.31 | 1.41 |
| | | 1 in Vehicle | | | CV % | −31.3 | 70.9 | −60.9 | 34.6 | 35.4 |
| | | Control | | | N | 12 | 12 | 12 | 12 | 12 |
| | | | | OS | Mean | −3.01 | 4.50 | −11.7 | 7.50 | 4.33 |
| | | | | | SD | 1.78 | 3.06 | 8.54 | 0.905 | 1.30 |
| | | | | | CV % | −59.0 | 68.0 | −73.2 | 12.1 | 30.1 |
| | | | | | N | 12 | 12 | 12 | 12 | 12 |
| | | | | OU | Mean | −2.36 | 4.58 | −8.58 | 6.67 | 4.00 |
| | | | | | SD | 0.940 | 2.84 | 5.34 | 2.31 | 1.81 |
| | | | | | CV % | −39.8 | 62.0 | −62.2 | 34.6 | 45.2 |
| | | | | | N | 12 | 12 | 12 | 12 | 12 |
| 4a | 1 | 0.005% | 35 | OD | Mean | −5.67 | 7.67 | −25.3 | 8.00 | 4.17 |
| | | Latanoprost | | | SD | 1.98 | 0.816 | 10.7 | 0.00 | 0.753 |
| | | in MF and | | | CV % | −34.9 | 10.6 | −42.2 | 0.00 | 18.1 |
| | | 4% | | | N | 6 | 6 | 6 | 6 | 6 |
| | | Compound | | OS | Mean | −5.78 | 6.00 | −24.4 | 8.00 | 3.83 |
| | | 1 in Vehicle | | | SD | 1.22 | 1.79 | 7.00 | 0.00 | 0.408 |
| | | Control | | | CV % | −21.2 | 29.8 | −28.7 | 0.00 | 10.6 |
| | | | | | N | 6 | 6 | 6 | 6 | 6 |
| | | | | OU | Mean | −5.64 | 7.00 | −24.6 | 8.00 | 4.33 |
| | | | | | SD | 1.41 | 1.67 | 8.09 | 0.00 | 0.516 |
| | | | | | CV % | −25.0 | 23.9 | −32.9 | 0.00 | 11.9 |
| | | | | | N | 6 | 6 | 6 | 6 | 6 |
| 4a | 5 | 0.005% | 35 | OD | Mean | −6.44 | 6.00 | −38.7 | 8.00 | 5.83 |
| | | Latanoprost | | | SD | 1.75 | 2.53 | 14.8 | 0.00 | 0.408 |
| | | in MF and | | | CV % | −27.1 | 42.2 | −38.3 | 0.00 | 7.00 |
| | | 4% | | | N | 6 | 6 | 6 | 6 | 6 |
| | | Compound | | OS | Mean | −6.44 | 5.67 | −35.2 | 8.00 | 5.67 |
| | | 1 in Vehicle | | | SD | 1.46 | 2.94 | 8.53 | 0.00 | 0.516 |
| | | Control | | | CV % | −22.6 | 52.0 | −24.2 | 0.00 | 9.11 |
| | | | | | N | 6 | 6 | 6 | 6 | 6 |
| | | | | OU | Mean | −6.31 | 5.67 | −36.9 | 8.00 | 5.67 |
| | | | | | SD | 1.72 | 2.94 | 11.4 | 0.00 | 0.516 |
| | | | | | CV % | −27.3 | 52.0 | −30.9 | 0.00 | 9.11 |
| | | | | | N | 6 | 6 | 6 | 6 | 6 |
| 4b | 1 | 0.005% | 35 | OD | Mean | −4.33 | 6.33 | −18.6 | 8.00 | 4.50 |
| | | Latanoprost | | | SD | 1.70 | 1.51 | 5.92 | 0.00 | 0.548 |
| | | in MF and | | | CV % | −39.2 | 23.8 | −31.8 | 0.00 | 12.2 |
| | | Vehicle | | | N | 6 | 6 | 6 | 6 | 6 |
| | | Control | | OS | Mean | −4.89 | 4.50 | −21.0 | 8.00 | 4.33 |
| | | | | | SD | 1.88 | 2.66 | 8.63 | 0.00 | 0.816 |
| | | | | | CV % | −38.5 | 59.2 | −41.1 | 0.00 | 18.8 |
| | | | | | N | 6 | 6 | 6 | 6 | 6 |
| | | | | OU | Mean | −4.50 | 4.50 | −19.6 | 8.00 | 4.33 |
| | | | | | SD | 1.76 | 2.66 | 6.87 | 0.00 | 0.816 |
| | | | | | CV % | −39.1 | 59.2 | −35.0 | 0.00 | 18.8 |
| | | | | | N | 6 | 6 | 6 | 6 | 6 |
| 4b | 5 | 0.005% | 35 | OD | Mean | −5.44 | 5.33 | −29.6 | 8.00 | 6.00 |
| | | Latanoprost | | | SD | 0.779 | 2.07 | 6.70 | 0.00 | 0.00 |
| | | in MF and | | | CV % | −14.3 | 38.7 | −22.6 | 0.00 | 0.00 |
| | | Vehicle | | | N | 6 | 6 | 6 | 6 | 6 |
| | | Control | | OS | Mean | −5.78 | 6.17 | −28.7 | 8.00 | 5.67 |
| | | | | | SD | 1.88 | 2.71 | 12.0 | 0.00 | 0.516 |
| | | | | | CV % | −32.6 | 44.0 | −41.8 | 0.00 | 9.11 |
| | | | | | N | 6 | 6 | 6 | 6 | 6 |
| | | | | OU | Mean | −5.36 | 4.50 | −28.9 | 8.00 | 6.00 |
| | | | | | SD | 1.29 | 2.66 | 9.18 | 0.00 | 0.00 |
| | | | | | CV % | −24.0 | 59.2 | −31.7 | 0.00 | 0.00 |
| | | | | | N | 6 | 6 | 6 | 6 | 6 |

TABLE 8

| | | | Day 1 | | | | | Day 5 | | | | |
|---|---|---|---|---|---|---|---|---|---|---|---|---|
| | Test | | Eₘₐₓ | Eₘₐₓ | Tₘₐₓ | AUEC₀₋ₜ | AUEC₀₋ₜ | Eₘₐₓ | Eₘₐₓ | Tₘₐₓ | AUEC₀₋ₜ | AUEC₀₋ₜ |
| Phase | Article | | (%) | (mmHg) | (h) | (h*%) | (h*mmHg) | (%) | (mmHg) | (h) | (h*%) | (h*mmHg) |
| 1 | Vehicle | Mean | 10.8 | −1.85 | 1.36 | 33.2 | −5.36 | NA | NA | NA | NA | NA |
| | Control | SD | 5.19 | 0.895 | 1.12 | 22.1 | 3.57 | NA | NA | NA | NA | NA |
| | | CV % | 48.0 | −48.4 | 82.1 | 66.6 | −66.5 | NA | NA | NA | NA | NA |
| | | N | 12 | 12 | 11 | 11 | 11 | 0 | 0 | 0 | 0 | 0 |
| 2 | 0.005% | Mean | 26.2 | −4.17 | 6.67 | 126 | −20.2 | NA | NA | NA | NA | NA |
| | Latanoprost | SD | 9.43 | 1.81 | 1.56 | 72.9 | 12.8 | NA | NA | NA | NA | NA |
| | in MF | CV % | 36.0 | −43.5 | 23.4 | 58.0 | −63.0 | NA | NA | NA | NA | NA |
| | | N | 12 | 12 | 12 | 12 | 12 | 0 | 0 | 0 | 0 | 0 |
| 3 | 4% | Mean | 17.3 | −3.58 | 4.92 | 70.3 | −13.7 | 13.8 | −2.36 | 5.08 | 51.1 | −8.58 |
| | Compound 1 | SD | 9.38 | 1.58 | 3.00 | 49.7 | 6.94 | 4.95 | 0.940 | 2.87 | 30.1 | 5.34 |
| | in Vehicle | CV % | 54.3 | −44.2 | 61.0 | 70.6 | −50.6 | 35.8 | −39.8 | 56.6 | 58.9 | −62.2 |
| | Control | N | 12 | 12 | 12 | 12 | 12 | 12 | 12 | 12 | 12 | 12 |
| 4a | 0.005% | Mean | 34.7 | −5.64 | 7.67 | 151 | −24.6 | 38.3 | −6.31 | 8.00 | 226 | −36.9 |
| | Latanoprost | SD | 8.62 | 1.41 | 0.816 | 44.0 | 8.09 | 7.46 | 1.72 | 0.00 | 65.7 | 11.4 |
| | in MF and 4% | CV % | 24.9 | −25.0 | 10.6 | 29.2 | −32.9 | 19.5 | −27.3 | 0.00 | 29.1 | −30.9 |
| | Compound 1 | N | 6 | 6 | 6 | 6 | 6 | 6 | 6 | 6 | 6 | 6 |
| | in Vehicle | | | | | | | | | | | |
| | Control | | | | | | | | | | | |
| 4b | 0.005% | Mean | 28.7 | −4.50 | 6.00 | 130 | −19.6 | 34.1 | −5.36 | 5.33 | 193 | −28.9 |
| | Latanoprost | SD | 12.0 | 1.76 | 1.26 | 41.0 | 6.87 | 8.30 | 1.29 | 2.07 | 42.2 | 9.18 |
| | in MF and | CV % | 41.8 | −39.1 | 21.1 | 31.6 | −35.0 | 24.4 | −24.0 | 38.7 | 21.8 | −31.7 |
| | Vehicle | N | 6 | 6 | 6 | 6 | 6 | 6 | 6 | 6 | 6 | 6 |
| | Control | | | | | | | | | | | |

As summarized in TABLE 9, Eₘₐₓ and AUC values indicate Compound 1 augments Latanoprost IOP decreases after TO administration in normotensive dogs.

TABLE 9

| | | Eₘₐₓ | Eₘₐₓ | Tₘₐₓ | AUEC₀₋ₜ | AUEC₀₋ₜ |
|---|---|---|---|---|---|---|
| Test Article | | (mmHg) | (%) | (h) | (h*mmHg) | (h*%) |
| Vehicle Control | Mean | −1.85 ± 0.895 | 10.8 ± 5.19 | 1.00 ± 1.18 | −5.36 ± 3.57 | 33.2 ± 22.1 |
| (15% HPβCD) | SD | | | | | |
| 4% Compound 1 | Mean | −2.36 ± 0.94 | 13.8 ± 4.95 | 4.58 ± 2.84 | −8.58 ± 5.34 | 51.1 ± 30.1 |
| | SD | | | | | |
| 0.005% Latanoprost + | Mean | −5.36 ± 1.29 | 34.1 ± 8.3 | 4.50 ± 2.66 | −28.9 ± 9.18 | 193 ± 42.2 |
| Vehicle Control | SD | | | | | |
| 0.005% Latanoprost + | Mean | −6.31 ± 1.72 | 38.3 ± 7.46 | 5.67 ± 2.94 | −36.9 ± 11.4 | 226 ± 65.7 |
| 4% Compound 1 | SD | | | | | |

No consistent differences in IOP were observed between the left or right eye for any phase; therefore, results and discussion are based on combined eye values.

After topical ocular administration of different formulations for each phase of the study, reduced IOP was observed, with mean Tₘₐₓ values for corrected IOP (mmHg) of 1.00 hours following administration of the vehicle control in Phase 1, of 6.17 hours following administration of 0.005% Latanoprost in marketed formulation (MF, Xalatan®) in Phase 2, and of 3.92 and 4.58 hours (respectively, for Days 1 and 5) following administration of 4% Compound 1 in vehicle control in Phase 3. After pre-treatment with 0.005% Latanoprost in MF and administration of vehicle control (referred to as Phase 4a, Animals D0001 through D0006) or 4% Compound 1 in vehicle control (referred to as Phase 4b, Animals D0007 through D0012), reduced IOP was also observed, with mean Tₘₐₓ values of 7.00 hours following a single dose and 5.67 hours following five daily dosings of vehicle control, and mean Tₘₐₓ values of 4.50 hours following a single dose or following five daily dosings of 4% Compound 1 in vehicle control.

Following a single topical ocular administration, the largest decrease in IOP, as assessed by Eₘₐₓ and AUEC of baseline-corrected or percent change from baseline IOP values, was observed with administration of 0.005% Latanoprost and 4% Compound 1 (Phase 4a); followed by administration of 0.005% Latanoprost either alone (Phase 2) or with vehicle control (Phase 4b); then by administration of 4% Compound 1 alone (Phase 3); and lastly administration of vehicle control (Phase 1). Following 5 days of dosing, the IOP-lowering effects of Compound 1 alone (Phase 3) and of 0.005% Latanoprost immediately followed by vehicle or Compound 1 (Phase 4) remained consistent with the respective IOP-lowering effect on Day 1. In addition, the corrected IOP measured at time 0 on Day 5 was lower than as observed at time 0 on Day 1 for Phases 4a. This result suggests that IOP lowering persists for at least 24 hours post-dose of Compound 1 when co-administered with Latanoprost.

Following topical ocular administration of the vehicle control (Phase 1), a slight decrease in IOP was observed as assessed by corrected IOP values and percentage of change from baseline, and with generally, slightly lower corrected IOP values at times 0 and 1 hour post-dose relative to values for the other treatment phases. This observation suggests that baseline corrected IOP parameter values for vehicle control may have resulted in an underestimation of effectiveness for the other treatment phases relative to vehicle control. Overall, following topical ocular administration of the vehicle control, a slight decrease in IOP was observed, with Eₘₐₓ values of 10.8%/−1.85 mmHg and AUEC₀₋ₜ of 33.2 h·%/−5.36 h·mmHg. Following a single topical ocular administration of 4% AKB-9778 in vehicle control, a greater decrease in IOP was observed compared with that following administration of vehicle control alone, with Eₘₐₓ values of 17.3%/−3.58 mmHg and AUEC₀₋ₜ of 70.3 h·%/−13.7 h·mmHg. Following a single (Day 1) topical ocular administration of 0.005% Latanoprost in MF and 4% Compound 1 in vehicle control, a greater decrease in IOP (Eₘₐₓ values of 34.7%/−5.64 mmHg and AUEC₀₋ₜ of 151 h·%/−24.6 h·mmHg) was observed compared with that following administration of 0.005% Latanoprost in MF and vehicle control (Eₘₐₓ values of 28.7%/−4.50 mmHg and AUEC₀₋ₜ of 130 h·%/−19.6% h·mmHg.

### CONCLUSIONS

A repeated topical ocular administration of 4% Compound 1 alone resulted in conjunctival hyperemia 21 of 24 eyes (1+ in 13 eyes and 2+ in 8 eyes observed on Day 4), but no discernable changes in pupil size or responsiveness to tropicamide. Topical ocular administration of 0.005% Latanoprost with vehicle control or in combination with 4% Compound 1 resulted in intense miosis and failure of the pupil to dilate following topical ocular application of tropicamide. Conjunctival hyperemia scores in eyes administered 0.005% Latanoprost and vehicle control (1+ in 10 eyes, 2+ in two eyes) were less than those observed in eyes administered 0.005% Latanoprost in combination with 4% Compound 1 (1+ in 4 eyes, 2+ in 6 eyes, and 3+ in 2 eyes). Conjunctival hyperemia scores in eyes administered 0.005% Latanoprost and 4% Compound 1 were also greater than those observed when either drug was administered alone (4% Compound 1) or with vehicle control.

Following a single topical ocular administration, the largest decrease in IOP, as assessed by Eₘₐₓ and AUEC of baseline-corrected or percent change from baseline IOP values, was observed with administration of 0.005% Latanoprost and 4% Compound 1 (Phase 4a); followed by administration of 0.005% Latanoprost either alone (Phase 2) or with vehicle control (Phase 4b); then by administration of 4% Compound 1 alone (Phase 3); and lastly administration of vehicle control (Phase 1). Following 5 days of dosing, the IOP-lowering effects of Compound 1 alone (Phase 3) and of 0.005% Latanoprost immediately followed by vehicle or Compound 1 (Phase 4) remained consistent with the respective IOP-lowering effect on Day 1. In addition, the corrected IOP measured at time 0 on Day 5 was lower than time 0 on Day 1 for Phase 4a, suggesting that IOP lowering persists for at least 24 hours post-dose of Compound 1 when co-administered with Latanoprost.

Following topical ocular administration of the vehicle control (Phase 1), a slight decrease in IOP was observed as assessed by corrected IOP values and percentage of change from baseline; and with generally, slightly lower corrected IOP values at times 0 and 1 hour post-dose relative to values for the other treatment phases. This observation suggests that baseline corrected IOP parameter values for vehicle control may have resulted in an underestimation of effectiveness for the other treatment phases relative to vehicle control.

Overall, following topical ocular administration of the vehicle control, a slight decrease in IOP was observed, with Eₘₐₓ values of 10.8%/−1.85 mmHg and AUEC₀₋ₜ of 33.2 h·%/−5.36 h·mmHg. Following a single topical ocular administration of 4% Compound 1 in vehicle control, a greater decrease in IOP was observed compared with that following administration of vehicle control alone, with Eₘₐₓ values of 17.3%/−3.58 mmHg and AUEC₀₋ₜ of 70.3 h·%/−13.7 h·mmHg. Following a single (Day 1) topical ocular administration of 0.005% Latanoprost in MF and 4% Compound 1 in vehicle control, a greater decrease in IOP (Eₘₐₓ values of 34.7%/−5.64 mmHg and AUEC₀₋ₜ of 151 h·%/−24.6 h·mmHg) was observed compared with that following administration of 0.005% Latanoprost in MF and vehicle control (Eₘₐₓ values of 28.7%/−4.50 mmHg and AUEC₀₋ₜ of 130 h·%/−19.6% h·mmHg.

### EMBODIMENTS

The following non-limiting embodiments provide illustrative examples of methods of the disclosure but do not limit the scope of methods of the disclosure.

Embodiment 1. A method of reducing intraocular pressure in a subject in need thereof, the method comprising: administering to the subject a therapeutically-effective amount of a Tie-2 activator; and administering to the subject a therapeutically-effective amount of a compound that causes agonism of a prostaglandin receptor, wherein the Tie-2 activator is not an angiopoietin.

Embodiment 2. The method of embodiment 1, wherein the Tie-2 activator and the compound that causes agonism of the prostaglandin receptor are co-formulated in a unit form dosage.

Embodiment 3. The method of embodiment 1, wherein the administering of the Tie-2 activator and the administering of the compound that causes agonism of the prostaglandin receptor are at least 5 minutes apart.

Embodiment 4. The method of embodiment 1, wherein the administering of the Tie-2 activator and the administering of the compound that causes agonism of the prostaglandin receptor are about 5 minutes apart.

Embodiment 5. The method of any one of embodiments 1-4, wherein the administering of the Tie-2 activator is topical.

Embodiment 6. The method of any one of embodiments 1-5, wherein the administering of the Tie-2 activator is once daily.

Embodiment 7. The method of any one of embodiments 1-5, wherein the administering of the Tie-2 activator is twice daily.

Embodiment 8. The method of any one of embodiments 1-7, wherein the administering of the Tie-2 activator is during morning.

Embodiment 9. The method of any one of embodiments 1-7, wherein the administering of the Tie-2 activator is during evening.

Embodiment 10. The method of any one of embodiments 1-9, wherein the therapeutically-effective amount of the Tie-2 activator is from about 0.1 mg to about 100 mg.

Embodiment 11. The method of any one of embodiments 1-9, wherein the therapeutically-effective amount of the Tie-2 activator is about 1 mg.

Embodiment 12. The method of any one of embodiments 1-9, wherein the therapeutically-effective amount of the Tie-2 activator is about 3 mg.

Embodiment 13. The method of any one of embodiments 1-12, wherein the Tie-2 activator is present in a composition at a concentration of from about 0.1 mg/mL to about 100 mg/mL.

Embodiment 14. The method of any one of embodiments 1-12, wherein the Tie-2 activator is present in a composition at a concentration of about 40 mg/mL.

Embodiment 15. The method of any one of embodiments 1-14, wherein the administering of the compound that causes agonism of the prostaglandin receptor is topical.

Embodiment 16. The method of any one of embodiments 1-15, wherein the administering of the compound that causes agonism of the prostaglandin receptor is once daily.

Embodiment 17. The method of any one of embodiments 1-15, wherein the administering of the compound that causes agonism of the prostaglandin receptor is twice daily.

Embodiment 18. The method of any one of embodiments 1-17, wherein the administering of the compound that causes agonism of the prostaglandin receptor is during evening.

Embodiment 19. The method of any one of embodiments 1-18, wherein the compound that causes agonism of the prostaglandin receptor is present in a formulation at a concentration of about 50 μg/mL.

Embodiment 20. The method of any one of embodiments 1-19, wherein the prostaglandin receptor is a prostaglandin F receptor.

Embodiment 21. The method of any one of embodiments 1-20, wherein the prostaglandin receptor is a prostaglandin F2 receptor.

Embodiment 22. The method of any one of embodiments 1-21, wherein the prostaglandin receptor is a prostaglandin F2 alpha receptor.

Embodiment 23. The method of any one of embodiments 1-22, wherein the compound that causes agonism of the prostaglandin receptor is a prostaglandin analogue.

Embodiment 24. The method of any one of embodiments 1-23, wherein the compound that causes agonism of the prostaglandin receptor is a prostaglandin F2 alpha analogue.

Embodiment 25. The method of any one of embodiments 1-24, wherein the compound that causes agonism of the prostaglandin receptor is an isopropyl ester.

Embodiment 26. The method of any one of embodiments 1-25, wherein the compound that causes agonism of the prostaglandin receptor is latanoprost.

Embodiment 27. The method of any one of embodiments 1-25, wherein the compound that causes agonism of the prostaglandin receptor is latanoprostene bunod.

Embodiment 28. The method of any one of embodiments 1-25, wherein the compound that causes agonism of the prostaglandin receptor is travoprost.

Embodiment 29. The method of any one of embodiments 1-25, wherein the compound that causes agonism of the prostaglandin receptor is tafluprost.

Embodiment 30. The method of any one of embodiments 1-25, wherein the compound that causes agonism of the prostaglandin receptor is unoprostone isopropyl.

Embodiment 31. The method of any one of embodiments 1-24, wherein the compound that causes agonism of the prostaglandin receptor is a prostamide.

Embodiment 32. The method of any one of embodiments 1-24 and 31, wherein the compound that causes agonism of the prostaglandin receptor is bimatoprost.

Embodiment 33. The method of any one of embodiments 1-30, wherein the compound that causes agonism of the prostaglandin receptor is a prodrug of a prostaglandin receptor agonist.

Embodiment 34. The method of embodiment 33, wherein the prostaglandin receptor agonist is latanoprost acid.

Embodiment 35. The method of embodiment 33, wherein the prostaglandin receptor agonist is travoprost acid.

Embodiment 36. The method of embodiment 33, wherein the prostaglandin receptor agonist is tafluprost acid.

Embodiment 37. The method of embodiment 33, wherein the prostaglandin receptor agonist is unoprostone acid.

Embodiment 38. The method of embodiment 33, wherein the compound that causes agonism of the prostaglandin receptor is hydrolyzed to an acid in the subject.

Embodiment 39. The method of embodiment 33, wherein the compound that causes agonism of the prostaglandin receptor is hydrolyzed by an esterase in an eye of the subject.

Embodiment 40. The method of embodiment 33, wherein the compound that causes agonism of the prostaglandin receptor is activated by hydrolysis.

Embodiment 41. The method of embodiment 33, wherein the compound that causes agonism of the prostaglandin receptor is activated by hydrolysis to an acid.

Embodiment 42. The method of any one of embodiments 1-41, wherein the compound that causes agonism of the prostaglandin receptor is absorbed through a cornea of an eye of the subject.

Embodiment 43. The method of any one of embodiments 1-42, wherein the compound that causes agonism of the prostaglandin receptor is absorbed through a sclera of an eye of the subject.

Embodiment 44. The method of any one of embodiments 1-43, wherein the administering of the compound that causes agonism of the prostaglandin receptor reduces intraocular pressure in the subject by increasing outflow of aqueous fluid from an eye of the subject.

Embodiment 45. The method of any one of embodiments 1-44, wherein the administering of the compound that causes agonism of the prostaglandin receptor reduces intraocular pressure in the subject by increasing permeability of a sclera of an eye of the subject.

Embodiment 46. The method of any one of embodiments 1-45, wherein the administering of the Tie-2 activator and administering of the compound that causes agonism of the prostaglandin receptor treats a condition in the subject.

Embodiment 47. The method of embodiment 46, wherein the condition is an ocular condition.

Embodiment 48. The method of embodiment 47, wherein the ocular condition is elevated intraocular pressure.

Embodiment 49. The method of embodiment 47, wherein the ocular condition is ocular hypertension.

Embodiment 50. The method of embodiment 47, wherein the ocular condition is glaucoma.

Embodiment 51. The method of any one of embodiments 1-50, wherein the Tie-2 activator is a compound of the formula:

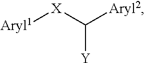

wherein:
Aryl¹ is an aryl group which is substituted or unsubstituted; Aryl² is an aryl group which is substituted or unsubstituted; X is alkylene, alkenylene, alkynylene, an ether linkage, an amine linkage, an amide linkage, an ester linkage, a thioether linkage, a carbamate linkage, a carbonate linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or

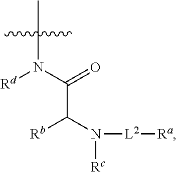

wherein:
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L²,
R^{a}, R^{b}, and R^{d} forms a ring that is substituted or unsubstituted;
R^{d} is H or alkyl which is substituted or unsubstituted, or together with any of L², R^{a}, R^{b}, and R^{C} forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted,
   or a pharmaceutically-acceptable salt thereof.

Embodiment 52. The method of embodiment 51, wherein:
Aryl¹ is substituted or unsubstituted phenyl;
Aryl² is substituted or unsubstituted heteroaryl; and
X is alkylene.

Embodiment 53. The method of embodiment 51, wherein Aryl² is:

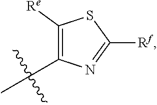

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

Embodiment 54. The method of embodiment 53, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R^{f} is heteroaryl or alkyl.

Embodiment 55. The method of any one of embodiments 1-54, wherein the Tie-2 activator is:

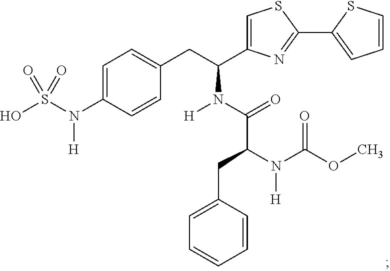 [SMILES: COC(=O)N[C@@H](Cc1ccccc1)C(=O)N[C@@H](Cc1ccc(NS(=O)(=O)O)cc1)c1csc(-c2cccs2)n1]

or a pharmaceutically-acceptable salt thereof.

Embodiment 56. The method of any one of embodiments 1-54, wherein the Tie-2 activator is:

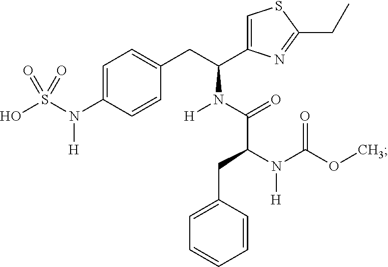 [SMILES: CCc1nc([C@H](Cc2ccc(NS(=O)(=O)O)cc2)NC(=O)[C@H](Cc2ccccc2)NC(=O)OC)cs1]

or a pharmaceutically-acceptable salt thereof.

Embodiment 57. The method of embodiment 51 or 52, wherein Aryl² is:

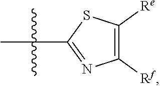

wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

Embodiment 58. The method of embodiment 57, wherein:
Aryl¹ is 4-phenylsulfamic acid;
R^{a} is alkyl, which is substituted or unsubstituted;
R^{b} is arylalkyl, which is substituted or unsubstituted;
R^{e} is H; and
R is heteroaryl or alkyl.

Embodiment 59. The method of any one of embodiments 1-52, 57, and 58, wherein the Tie-2 activator is:

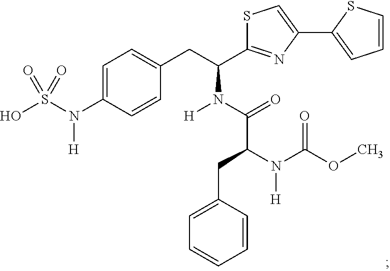 [SMILES: COC(=O)N[C@@H](Cc1ccccc1)C(=O)N[C@@H](Cc1ccc(NS(=O)(=O)O)cc1)c1nc(-c2cccs2)cs1]

or a pharmaceutically-acceptable salt thereof.

Embodiment 60. The method of any one of embodiments 1-52, 57, and 58, wherein the Tie-2 activator is:

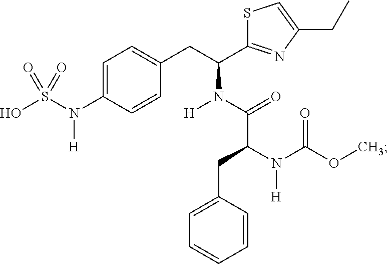 [SMILES: CCc1csc([C@H](Cc2ccc(NS(=O)(=O)O)cc2)NC(=O)[C@H](Cc2ccccc2)NC(=O)OC)n1]

or a pharmaceutically-acceptable salt thereof.

Embodiment 61. A method of reducing intraocular pressure in a subject in need thereof, the method comprising: administering to an eye of the subject a therapeutically-effective amount of a Tie-2 activator of the formula:

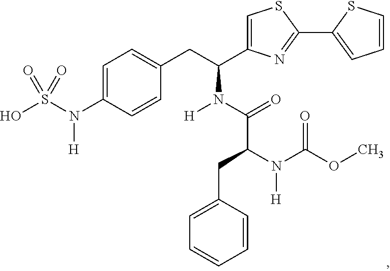 [SMILES: COC(=O)N[C@@H](Cc1ccccc1)C(=O)N[C@@H](Cc1ccc(NS(=O)(=O)O)cc1)c1csc(-c2cccs2)n1]

or a pharmaceutically-acceptable salt thereof; and administering to the eye of the subject a therapeutically-effective amount of latanoprost,
wherein the Tie-2 activator is administered as part of a composition at a concentration of about 40 mg/mL.
wherein the Tie-2 activator is topically administered to the eye of the subject,
wherein the Tie-2 activator is administered once daily,
wherein the latanoprost is administered as part of a formulation having a concentration of about 50 μg/mL,
wherein the latanoprost is topically administered to the eye of the subject,
wherein the latanoprost is administered once daily,
wherein the administering of the Tie-2 activator and the administering of the latanoprost reduces intraocular pressure in the subject by about 1 mmHg to about 5 mmHg.

Embodiment 62. The method of embodiment 61, wherein the administering of the Tie-2 activator and the administering of the latanoprost are at least 5 minutes apart.

Embodiment 63. The method of embodiment 61, wherein the administering of the Tie-2 activator and the administering of the latanoprost are about 5 minutes apart.

Embodiment 64. The method of any one of embodiments 61-63, wherein the administering of the Tie-2 activator is during morning.

Embodiment 65. The method of any one of embodiments 61-63, wherein the administering of the Tie-2 activator is during evening.

Embodiment 66. The method of any one of embodiments 61-65, wherein the administering of the latanoprost is during evening.

Embodiment 67. The method of any one of embodiments 61-66, wherein the administering of the Tie-2 activator and administering of the latanoprost treat a condition in the subject.

Embodiment 68. The method of embodiment 67, wherein the condition is an ocular condition.

Embodiment 69. The method of embodiment 68, wherein the ocular condition is elevated intraocular pressure.

Embodiment 70. The method of embodiment 68, wherein the ocular condition is ocular hypertension.

Embodiment 71. The method of embodiment 68, wherein the ocular condition is glaucoma.

Embodiment 72. A kit comprising: a) a Tie-2 activator; b) a compound that causes agonism of a prostaglandin receptor; and c) written instructions on use of the kit for treatment of a condition, wherein the Tie-2 activator is not an angiopoietin.

Embodiment 73. A kit comprising: a) a container, wherein the container is a dropper bottle; and b) a dosage form contained in the dropper bottle, wherein the unit dosage form comprises a Tie-2 activator and a compound that causes agonism of a prostaglandin receptor, wherein the Tie-2 activator is not an angiopoietin.

Embodiment 74. The kit of embodiment 73, wherein the method further comprises written instructions for administering the unit dosage form to the subject by topical drop.

Embodiment 75. A method of reducing intraocular pressure in a subject in need thereof, the method comprising: administering to the subject a therapeutically-effective amount of a Tie-2 activator; and a compound that is timolol, befunolol, betaxolol, carteolol, levobunolol, metipranolol, mepindolol, acetazolamide, brinzolamide, diclofenamide, dorzolamide, methazolamide, epinephrine, brimonidine, apraclonidine, ripasudil, netarsudil, pilocarpine, carbachol, or echothiophate iodide, wherein the Tie-2 activator is not an angiopoietin.

Embodiment 76. A method of reducing intraocular pressure in a subject in need thereof, the method comprising: administering to the subject a therapeutically-effective amount of a Tie-2 activator; and administering to the subject a therapeutically-effective amount of a compound that causes agonism of a prostaglandin receptor, wherein the Tie-2 activator is a phosphatase binding agent.

Embodiment 77. A method of reducing intraocular pressure in a subject in need thereof, the method comprising: administering to the subject a therapeutically-effective amount of a Tie-2 activator; and administering to the subject a therapeutically-effective amount of a compound that causes agonism of a prostaglandin receptor, wherein the Tie-2 activator is a protein tyrosine phosphatase binding agent.

Embodiment 78. A method of reducing intraocular pressure in a subject in need thereof, the method comprising: administering to the subject a therapeutically-effective amount of a Tie-2 activator; and administering to the subject a therapeutically-effective amount of a compound that causes agonism of a prostaglandin receptor, wherein the Tie-2 activator is a VE-PTP binding agent.

Embodiment 79. A method of reducing intraocular pressure in a subject in need thereof, the method comprising: administering to the subject a therapeutically-effective amount of a Tie-2 activator; and administering to the subject a therapeutically-effective amount of a compound that causes agonism of a prostaglandin receptor, wherein the Tie-2 activator is a phosphatase inhibitor.

Embodiment 80. A method of reducing intraocular pressure in a subject in need thereof, the method comprising: administering to the subject a therapeutically-effective amount of a Tie-2 activator; and administering to the subject a therapeutically-effective amount of a compound that causes agonism of a prostaglandin receptor, wherein the Tie-2 activator is a VE-PTP inhibitor.

Embodiment 81. A method of reducing intraocular pressure in a subject in need thereof, the method comprising: administering to the subject a therapeutically-effective amount of a Tie-2 activator; and administering to the subject a therapeutically-effective amount of a compound that causes agonism of a prostaglandin receptor, wherein the Tie-2 activator is a protein tyrosine phosphatase inhibitor.

Embodiment 82. A method of reducing intraocular pressure in a subject in need thereof, the method comprising: administering to the subject a therapeutically-effective amount of a Tie-2 activator; and administering to the subject a therapeutically-effective amount of a compound that causes agonism of a prostaglandin receptor, wherein the reduction in intraocular pressure of the subject after administration is greater than the reduction in intraocular pressure of the subject after administration of either the Tie-2 activator or the compound that causes agonism of a prostaglandin receptor alone.

Embodiment 83. A method of reducing intraocular pressure in a subject in need thereof, the method comprising: administering to the subject a therapeutically-effective amount of a Tie-2 activator; and administering to the subject a therapeutically-effective amount of a compound that causes agonism of a prostaglandin receptor, wherein the intraocular pressure of the subject reduces by at least 2 mmHg after administration.

Embodiment 84. A method of reducing intraocular pressure in a subject in need thereof, the method comprising: administering to the subject a therapeutically-effective amount of a Tie-2 activator; and administering to the subject a therapeutically-effective amount of a compound that causes agonism of a prostaglandin receptor, wherein the reduction in intraocular pressure of the subject persists for at least 24 hours after administration.

Embodiment 85. A method of reducing intraocular pressure in a subject in need thereof, the method comprising: administering to the subject a therapeutically-effective amount of a HPTPβ inhibitor; and administering to the subject a therapeutically-effective amount of a compound that causes agonism of a prostaglandin receptor.

1. A method of reducing intraocular pressure in a subject in need thereof, the method comprising:
administering to the subject a therapeutically-effective amount of a Tie-2 activator; and
administering to the subject a therapeutically-effective amount of a compound that causes agonism of a prostaglandin receptor, wherein the Tie-2 activator is not an angiopoietin.

2. (canceled)

3. The method of claim 1, wherein the administering of the Tie-2 activator and the administering of the compound that causes agonism of the prostaglandin receptor are at least 5 minutes apart.

4. (canceled)

5. The method of claim 1, wherein the administering of the Tie-2 activator is topical.

6. The method of claim 1, wherein the administering of the Tie-2 activator is once daily.

7. The method of claim 1, wherein the administering of the Tie-2 activator is twice daily.

8-9. (canceled)

10. The method of claim 1, wherein the therapeutically-effective amount of the Tie-2 activator is from about 0.1 mg to about 100 mg.

11. The method of claim 1, wherein the therapeutically-effective amount of the Tie-2 activator is about 1 mg to about 5 mg.

12. (canceled)

13. The method of claim 1, wherein the Tie-2 activator is present in a composition at a concentration of from about 0.1 mg/mL to about 100 mg/mL.

14. The method of claim 1, wherein the Tie-2 activator is present in a composition at a concentration of about 40 mg/mL.

15. The method of claim 1, wherein the administering of the compound that causes agonism of the prostaglandin receptor is topical.

16. The method of claim 1, wherein the administering of the compound that causes agonism of the prostaglandin receptor is once daily.

17-18. (canceled)

19. The method of claim 1, wherein the compound that causes agonism of the prostaglandin receptor is present in a formulation at a concentration of about 50 μg/mL.

20-25. (canceled)

26. The method of claim 1, wherein the compound that causes agonism of the prostaglandin receptor is latanoprost.

27-45. (canceled)

46. The method of claim 1, wherein the administering of the Tie-2 activator and administering of the compound that causes agonism of the prostaglandin receptor treats a condition in the subject.

47. The method of claim 46, wherein the condition is an ocular condition.

48. The method of claim 47, wherein the ocular condition is elevated intraocular pressure.

49. The method of claim 47, wherein the ocular condition is ocular hypertension.

50. The method of claim 47, wherein the ocular condition is glaucoma.

51. The method of claim 1, wherein the Tie-2 activator is a compound of the formula: 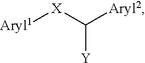
wherein:
Aryl¹ is an aryl group which is substituted or unsubstituted; Aryl² is an aryl group which is substituted or unsubstituted; X is alkylene, alkenylene, alkynylene, an ether linkage, an amine linkage, an amide linkage, an ester linkage, a thioether linkage, a carbamate linkage, a carbonate linkage, a sulfone linkage, any of which is substituted or unsubstituted, or a chemical bond; and Y is H, aryl, heteroaryl, NH(aryl), NH(heteroaryl), NHSO₂R^{g}, or NHCOR^{g}, any of which is substituted or unsubstituted, or 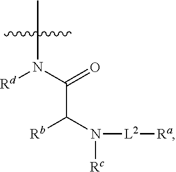
wherein:
L² is alkylene, alkenylene, or alkynylene, any of which is substituted or unsubstituted, or together with the nitrogen atom to which L² is bound forms an amide linkage, a carbamate linkage, or a sulfonamide linkage, or a chemical bond, or together with any of R^{a}, R^{b}, R^{c}, and R^{d} forms a ring that is substituted or unsubstituted;
R^{a} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{b}, R^{c}, and R^{d} forms a ring that is substituted or unsubstituted;
R^{b} is H, alkyl, alkenyl, alkynyl, aryl, arylalkyl, heterocyclyl, heterocyclylalkyl, heteroaryl, or heteroarylalkyl, any of which is substituted or unsubstituted, or together with any of L², R^{a}, R^{c}, and R^{d} forms a ring that is substituted or unsubstituted;
R^{c} is H or alkyl which is substituted or unsubstituted, or together with any of L², R^{a}, R^{b}, and R^{d} forms a ring that is substituted or unsubstituted;
R^{d} is H or alkyl which is substituted or unsubstituted, or together with any of L², R^{a}, R^{b}, and R^{C} forms a ring that is substituted or unsubstituted; and
R^{g} is H, alkyl, alkenyl, alkynyl, aryl, arylalkyl, heterocyclyl, heterocyclylalkyl, heteroaryl, or heteroarylalkyl, any of which is substituted or unsubstituted,
or a pharmaceutically-acceptable salt thereof.

52. The method of claim 51, wherein:
Aryl¹ is substituted or unsubstituted phenyl;
Aryl² is substituted or unsubstituted heteroaryl; and
X is alkylene.

53. The method of claim 51, wherein Aryl² is: 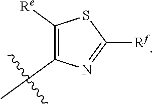
wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

54. (canceled)

55. The method of claim 1, wherein the Tie-2 activator is: 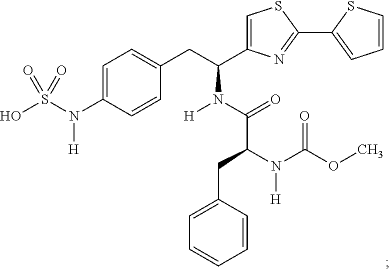 [SMILES: COC(=O)N[C@@H](Cc1ccccc1)C(=O)N[C@@H](Cc1ccc(NS(=O)(=O)O)cc1)c1csc(-c2cccs2)n1]
or a pharmaceutically-acceptable salt thereof.

56. The method of claim 1, wherein the Tie-2 activator is: 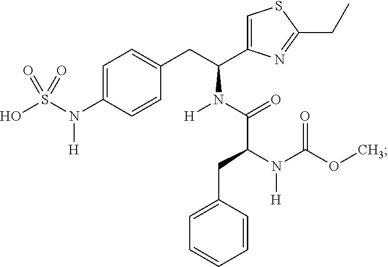 [SMILES: CCc1nc([C@H](Cc2ccc(NS(=O)(=O)O)cc2)NC(=O)[C@H](Cc2ccccc2)NC(=O)OC)cs1]
or a pharmaceutically-acceptable salt thereof.

57. The method of claim 51, wherein Aryl² is: 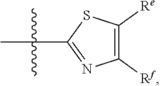
wherein:
R^{e} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted; and
R^{f} is H, OH, F, Cl, Br, I, CN, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, arylalkyl, heterocyclyl, heterocyclylalkyl, heteroaryl, or heteroarylalkyl, any of which is substituted or unsubstituted.

58. (canceled)

59. The method of claim 1, wherein the Tie-2 activator is: 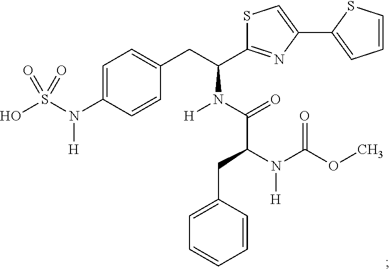 [SMILES: COC(=O)N[C@@H](Cc1ccccc1)C(=O)N[C@@H](Cc1ccc(NS(=O)(=O)O)cc1)c1nc(-c2cccs2)cs1]
or a pharmaceutically-acceptable salt thereof.

60. The method of claim 1, wherein the Tie-2 activator is: 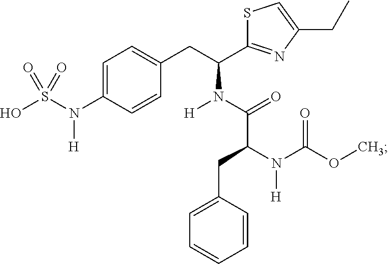 [SMILES: CCc1csc([C@H](Cc2ccc(NS(=O)(=O)O)cc2)NC(=O)[C@H](Cc2ccccc2)NC(=O)OC)n1]
or a pharmaceutically-acceptable salt thereof.

61. A method of reducing intraocular pressure in a subject in need thereof, the method comprising:
administering to an eye of the subject a therapeutically-effective amount of a Tie-2 activator of the formula: 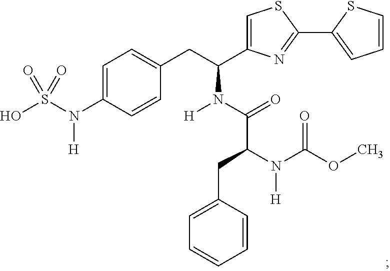 [SMILES: COC(=O)N[C@@H](Cc1ccccc1)C(=O)N[C@@H](Cc1ccc(NS(=O)(=O)O)cc1)c1csc(-c2cccs2)n1]
or a pharmaceutically-acceptable salt thereof; and
administering to the eye of the subject a therapeutically-effective amount of latanoprost,
wherein the Tie-2 activator is administered as part of a composition at a concentration of about 40 mg/mL,
wherein the Tie-2 activator is topically administered to the eye of the subject,
wherein the Tie-2 activator is administered once daily,
wherein the latanoprost is administered as part of a formulation having a concentration of about 50 μg/mL,
wherein the latanoprost is topically administered to the eye of the subject,
wherein the latanoprost is administered once daily, and
wherein the administering of the Tie-2 activator and the administering of the latanoprost reduces intraocular pressure in the subject by about 1 mmHg to about 5 mmHg.

62-74. (canceled)
